SEQUENCE LISTING (21 sequences)

SEQ ID NO: 1 - DNA, 1279 bp, Human
tccaaaacccgaggtctcgctaaaatcatcatggattcacttggcgccgtcagcactcgacttgggtttgatcttttcaaagagctgaagaaaacaaatgatggcaacatcttcttttcccctgtgggcatcttgactgcaattggcatggtcctcctggggacccgaggagccaccgctccccagttggaggaggtgtttcactctgaaaaagagacgaagagctcaagaataaaggctgaagaaaaagaggtgattgagaacacagaagcagtacatcaacaattccaaaagtttttgactgaaataagcaaactcactaatgattatgaactgaacataaccaacaggctgtttggagaaaaaacatacctcttccttcaaaaatacttagattatgttgaaaaatattatcatgcatctctggaacctgttgattttgtaaatgcagccgatgaaagtcgaaagaagattaattcctgggttgaaagcaaaacaaatgaaaaaatcaaggacttgttcccagatggctctattagtagctctaccaagctggtgctggtgaacatggtttattttaaagggcaatgggacagggagtttaagaaagaaaatactaaggaagagaaattttggatgaataagagcacaagtaaatctgtacagatgatgacacagagccattcctttagcttcactttcctggaggacttgcaggccaaaattctagggattccatataaaaacaacgacctaagcatgtttgtgcttctgcccaacgacatcgatggcctggagaagataatagataaaataagtcctgagaaattggtagagtggactagtccagggcatatggaagaaagaaaggtgaatctgcacttgccccggtttgaggtggaggacagttacgatctagaggcggtcctggctgccatggggatgggcgatgccttcagtgagcacaaagccgactactcgggaatgtcgtcaggctccgggttgtacgcccagaagttcctgcacagttcctttgtggcagtaactgaggaaggcaccgaggctgcagctgccaccggcataggctttactgtcacatccgccccaggtcatgaaaatgttcactgcaatcatcccttcctgttcttcatcaggcacaatgaatccaacagcatcctcttcttcggcagattttcttctccttaagatgatcgttgccatggcattgctgcttttagcaaaaaacaactaccagtggtactcatatgaatatgaaaat

SEQ ID NO: 2 - protein, 391 aa, Human [sequence omitted]

SEQ ID NO: 3 - protein, 22 aa, Human
EEGTEAAAATGIGFTVTSALGH

SEQ ID NO: 4 - protein, 22 aa, Human
EEGAEAAAATAVVGFGSSPTST

SEQ ID NO: 5 - protein, 22 aa, Human
EEGVEAAAATAVVVVELSSPST

SEQ ID NO: 6 - protein, 22 aa, Human
EEGTEAAAGTGGVMTGRTGHGG

SEQ ID NO: 7 - protein, 400 aa, Human
MDSLGAVSTRLGFDLFKELKKTNDGNIFFSPVGILTAIGMVLLGTRGATASQLEEVFHSEKETKSSRIKAEEKEVVRIKAEGKEIENTEAVHQQFQKFLTEISKLTNDYELNITNRLFGEKTYLFLQKYLDYVEKYYHASLEPVDFVNAADESRKKINSWVESKTNEKIKDLFPDGSISSSTKLVLVNMVYFKGQWDREFKKENTKEEKFWMNKSTSKSVQMMTQSHSFSFTFLEDLQAKILGIPYKNNDLSMFVLLPNDIDGLEKIIDKISPEKLVEWTSPGHMEERKVNLHLPRFEVEDSYDLEAVLAAMGMGDAFSEHRADYSGMSSGSGLYAQKFLHSSFVAVTEEGTEAAAATGIGFTVTSALGHENVHCNHPFLFFIRHNESNSILFFGRFSSP

SEQ ID NO: 8 - protein, 55 aa, Human
LEDLQAKILGIPYKNNDLSMFVLLPNDIDGLEKVNAYTSLFFLSFPKAFCLRASE

SEQ ID NO: 9 - protein, 390 aa, Human
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVVGFGSSPTSTNEEFHCNHPFLFFIRQNKTNSILFYGRFSSP

SEQ ID NO: 10 - protein, 390 aa, Human
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 11 - protein, 415 aa, Human
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGDYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITKCILFFGRFCSP

SEQ ID NO: 12 - protein, 8 aa, Human
GTEAAAAT

SEQ ID NO: 13 - protein, 9 aa, Human
VRIKAEGKE

SEQ ID NO: 14 - DNA, 20 bp, Artificial Sequence
  Description of Artificial Sequence  Synthetic Primer
gtccagggcatatggaagaa

SEQ ID NO: 15 - DNA, 21 bp, Artificial Sequence
  Description of Artificial Sequence  Synthetic Primer
gggatgattgcagtgaacatt

SEQ ID NO: 16 - DNA, 405 bp, Human
tcctggaggacttgcaggccaaaattctagggattccatataaaaacaacgacctaagcatgtttgtgcttctgcccaacgacatcgatggcctggagaaggtaaacgcttacacctccttattctttctttcatttcctaaggctttttgtctcagggcttctgagtaggagctggtggccagcagtgtcaaatagaaagtgtttctcactctccagcagctacagatggatgtctactggggagcattaaatagtatgggtcaggtttattgagaacaactaatcctagaatgtttgggatgaactaaaatgaacatttaagattatatgggagtcctaacgatcacccaccattctgagaaatggttggtatctatggacccctttcctggaaaaaaaaaaa

SEQ ID NO: 17 - protein, 8 aa, Artificial Sequence
  Description of Artificial Sequence  Synthetic Peptide
GTXAAAAT

SEQ ID NO: 18 - DNA, 700 bp, Human
tccaaaacccgaggtctcgctaaaatcatcatggattcacttggcgccgtcagcactcgacttgggtttgatcttttcaaagagctgaagaaaacaaatgatggcaacatcttcttttcccctgtgggcatcttgactgcaattggcatggtcctcctggggacccgaggagccaccgctccccagttggaggaggtgtttcactctgaaaaagagacgaagagctcaagaataaaggctgaagaaaaagaggtgattgagaacacagaagcagtacatcaacaattccaaaagtttttgactgaaataagcaaactcactaatgattatgaactgaacataaccaacaggctgtttggagaaaaaacatacctcttccttcaaaaatacttagattatgttgaaaaatattatcatgcatctctggaacctgttgattttgtaaatgcagccgatgaaagtcgaaagaagattaattcctgggttgaaagcaaaacaaatgaaaaaatcaaggacttgttcccagatggctctattagtagctctaccaagctggtgctggtgaacatggtttattttaaagggcaatgggacagggagtttaagaaagaaaatactaaggaagagaaattttggatgaataagagcacaagtaaatctgtacagatgatgacacagagccattcctttagcttcactt

SEQ ID NO: 19 - protein, 224 aa, Human
MDSLGAVSTRLGFDLFKELKKTNDGNIFFSPVGILTAIGMVLLGTRGATASQLEEVFHSEKETKSSRIKAEEKEVIENTEAVHQQFQKFLTEISKLTNDYELNITNRLFGEKTYLFLQKYLDYVEKYYHASLEPVDFVNAADESRKKINSWVESKTNEKIKDLFPDGSISSSTKLVLVNMVYFKGQWDREFKKENTKEEKFWMNKSTSKSVQMMTQSHSFSFTF

SEQ ID NO: 20 - DNA, 478 bp, Human
ataatagataaaataagtcctgagaaattggtagagtggactagtccagggcatatggaagaaagaaaggtgaatctgcacttgccccggtttgaggtggaggacagttacgatctagaggcggtcctggctgccatggggatgggcgatgccttcagtgagcacaaagccgactactcgggaatgtcgtcaggctccgggttgtacgcccagaagttcctgcacagttcctttgtggcagtaactgaggaaggcaccgaggctgcagctgccaccggcataggctttactgtcacatccgccccaggtcatgaaaatgttcactgcaatcatcccttcctgttcttcatcaggcacaatgaatccaacagcatcctcttcttcggcagattttcttctccttaagatgatcgttgccatggcattgctgcttttagcaaaaaacaactaccagtggtactcatatgaatatgaaaat

SEQ ID NO: 21 - protein, 135 aa, Homo sapiens
KIIDKISPEKLVEWTSPGHMEERKVNLHLPRFEVEDSYDLEAVLAAMGMGDAFSEHKADYSGMSSGSGLYAQKFLHSSFVAVTEEGTEAAAATGIGFTVTSAPGHENVHCNHPFLFFIRHNESNSILFFGRFSSP

(19) United States
(12) Patent Application Publication
(10) Pub. No.: US 2004/0180371 A1
(43) Pub. Date: Sep. 16, 2004

(54) Methods and compositions of a novel serine protease inhibitor

(75) Inventors: Gary Clayman, Houston, TX (US); Torahiko Nakashima, Fukuoka City (JP); Paul Spring, Lexington, KY (US)

Correspondence Address: Steven L. Highlander, Esq., FULBRIGHT & JAWORSKI L.L.P., 600 Congress Avenue, Suite 2400, Austin, TX, 78701, US

(73) Assignee: Board of Regents, The University of Texas System

(21) Appl. No.: 10/766,778

(22) Filed: Jan. 28, 2004

Related U.S. Application Data
(62) Division of application No. 09/653,464, filed on Aug. 31, 2000, now abandoned.
(60) Provisional application No. 60/151,776, filed on Aug. 31, 1999.

(51) Int. Cl. C12Q 1/68; C07H 21/04; C12N 9/99; C07K 16/40

(52) U.S. Cl. 435/6; 435/69.2; 435/184; 435/320.1; 435/325; 530/388.26; 536/23.2

(57) ABSTRACT

The present invention describes a novel gene encoding a novel protein termed headpin (for head and neck serpin) that is homologous to known serine protease inhibitors. The invention describes methods and compositions of the nucleic acids, encoded proteins, antibodies, pharmaceuticals, cancer treatments, diagnostics and screens for modulators of headpin.

### BACKGROUND OF THE INVENTION

The present application claims the benefit of co-pending U.S. Provisional Patent Application Serial No. 60/151,776 filed Aug. 31, 1999, the entire text of which is specifically incorporated by reference herein without disclaimer. The government owns rights in the present invention pursuant to grant numbers R29 DE11689-01A1, IP50DE11906-01, and NIH-NCI-CA 16672 from the National Institutes of Health. 1. Field of the Invention

The present invention relates generally to the fields of biology and biochemistry. More particularly, it concerns a novel serine protease inhibitor and its involvement with cancer. 2. Description of Related Art

Serine proteases are universally found in living organisms, fulfilling a wide variety of functions. Serine proteases (E.C. 3.4.21) are the sub-sub class of endopeptidases that use serine as the nucleophile in peptide bond cleavage (Barrett, 1986; Hartley, 1960). In many cases, the serine proteases have specific targets, where precursors are activated to biologically competent products to discharge their particular function. In other cases, there is a more generalized activity, where particular dipeptide sequences within larger proteins are subject to scission in the process of degradation.

Two superfamilies of serine proteases, i.e., the chymotrypsin superfamily and the Streptomyces subtilisin superfamily, have been observed to date (Barrett, 1986; James, 1976). Examples of serine proteases of the chymotrypsin superfamily include tissue-type plasminogen activator (t-PA), trypsin, trypsin-like protease, chymotrypsin, plasmin, elastase, urokinase (or urinary-type plasminogen activator (u-PA)), acrosin, activated protein C, Cl esterase, cathepsin G. chymase and proteases of the blood coagulation cascade including kallikrein, thrombin, and Factors VIIa, IXa, Xa, XIa and XIIa (Barrett, 1986; Strassburger et al, 1983; Dayhoff, 1972; Rosenberg, 1986). Some of the serine proteases of the chymotrypsin superfamily, including t-PA, plasmin, u-PA and the proteases of the blood coagulation cascade, are large molecules that contain, in addition to the serine protease catalytic domain, other structural domains responsible in part for regulation of their activity (Barrett, 1986; Gerard et al, 1986; Blasi et al., 1986).

Among important serine proteases are trypsin-like enzymes, such as trypsin, tryptase, thrombin, kallikrein, and factor Xa. The serine protease targets are associated with processes such as blood clotting, complement mediated lysis, the immune response, glomerulonephritis, pain sensing, inflammation, pancreatitis, cancer, regulating fertilization, bacterial infection and viral maturation. By inhibiting serine proteases which have high specificity for a particular target, one can inhibit in vivo numerous biological processes, which may have dramatic effects on a host.

Serine proteinase inhibitors (serpins) comprise a diverse group of proteins that form a superfamily already including more than 100 members, from such diverse organisms as viruses, plants and humans. Serpins have evolved over 500 million years and diverged phylogenetically into proteins with inhibitory function and non-inhibitory function (Hunt and Dayhoff, 1980). Non-inhibitory serpins such as ovalbumin lack protease inhibitory activity (Remold-O'Donnell, 1993). The primary function of serpin family members appears to be neutralizing overexpressed serine proteinase activity (Potempa et al., 1994). Serpins play a role in extracellular matrix remodeling, modulation of inflammatory response and cell migration (Potempa et al., 1994).

Serine protease inhibitors are divided into the following families: the bovine pancreatic trypsin inhibitor (Kunitz) family, also known as basic protease inhibitor (Ketcham et al., 1978); the Kazal family; the Streptomyces subtilisin inhibitor family; the serpin family; the soybean trypsin inhibitor (Kunitz) family; the potato inhibitor family; and the Bowman-Birk family (Laskowski et al., 1980; Read et al., 1986; Laskowski et al., 1987). Serine protease inhibitors belonging to the serpin family include the plasminogen activator inhibitors PAI-1, PAI-2 and PAI-3, Cl esterase inhibitor, alpha-2-antiplasmin, contrapsin, alpha-1-antitrypsin, antithrombin III, protease nexin I, alpha-1-antichymotrypsin, protein C inhibitor, heparin cofactor II and growth hormone regulated protein (Carrell et al., 1987; Sommer et al., 1987; Suzuki et al., 1987; Stump et al., 1986).

Many of the serine protease inhibitors have a broad specificity and are able to inhibit both the chymotrypsin superfamily of proteases, including the blood coagulation serine proteases, and the Streptomyces subtilisin superfamily of serine proteases (Laskowski et al., 1980). The inhibition of serine proteases by serpins has been reviewed in Travis et al. (1983); Carrell et al. (1985); and Sprengers et al. (1987). Crystallographic data are available for a number of intact inhibitors including members of the BPTI, Kazal, SSI, soybean trypsin and potato inhibitor families, and for a cleaved form of the serpin alpha-1-antitrypsin (Read et al., 1986). Despite the fact that these serine protease inhibitors are proteins of diverse size and sequence, the intact inhibitors studied to date all have in common a characteristic loop, termed the reactive site loop, extending from the surface of the molecule that contains the recognition sequence for the active site of the cognate serine protease (Levin et al., 1983). The structural similarity of the loops in the different serine protease inhibitors is remarkable (Papamokos et al., 1982). The specificity of each inhibitor is thought to be determined primarily by the identity of the amino acid that is immediately amino-terminal to the site of potential cleavage of the inhibitor by the serine protease. This amino acid, known as the P₁ site residue, is thought to form an acyl bond with the serine in the active site of the serine protease (Laskowski et al., 1980). Whether or not a serpin possesses inhibitory function depends strongly on the consensus sequence located in the hinge region of the reactive site loop near the carboxy-terminus of the coding region. Outside of the reactive site loop, the serine protease inhibitors of different families are generally unrelated structurally, although the Kazal family and Streptomyces subtilisin family of inhibitors display some structural and sequence similarity.

In humans, serpins may be present both intracellularly and extracellularly (Bartuski et al., 1997). Serpin families tend to cluster at specific chromosomal locations. The ov-serpin family clusters at 18q21.3 and includes maspin, SCCA1, SCCA2 and PAI-2 (Bartuski et al., 1997; Schneider et al., 1995). The 18q21-23 chromosomal region exhibits frequent site of loss of heterozygosity in head and neck carcinomas and other solid malignancies (Jones et al., 1997; Frank et al., 1997; Jonson et al., 1999). This also is the locus of maspin, which behaves as a tumor suppressor (Zou et al., 1994).

Localized proteolytic destruction of connective tissue is generally considered as a prerequisite for tumor cell invasion. For instance, the secretion of plasminogen-activator (PA) by endothelial cells, leading to production of plasmin, has been proposed as a mechanism for initiating this process (Ossowski and Reich, 1983). The onset of PA synthesis has also been associated with a variety of neoplasia (Unkeless et al., 1980). Antibodies directed to PA have been shown to inhibit tumor cell metastasis (Ossowski and Reich, 1983; Mignatti et al., 1986). It is reasonable to assume that inhibitors of PA will inhibit both tumor metastasis and growth (Dvorak, 1986).

Because of the critical role serine protease inhibitors play in a number of biological systems, and in particular in cancer progression, migration and metastasis, a better understanding of these systems is critical. Novel serine protease inhibitors, especially those expressed in tissue specific or developmentally regulated manners, are of particular interest.

### SUMMARY OF THE INVENTION

The present invention overcomes the deficiencies in the art by providing a novel gene encoding a novel protein termed headpin (for head and neck serpin) that is homologous to known serine protease inhibitors. Headpin message is under expresssed in cancers such as, for example, head and neck cancers, and cervical cancer, and the thus provides diagnostic ability for cancer based detection of headpin message and protein expression levels. The invention further provides therapeutic compositions of nucleic acids that express headpin proteins, polypeptides, and peptides, as well as therapeutic compositions that comprise headpin proteins, polypeptides and peptides, and biologically functional equivalents thereof. The invention further describes methods and compositions of the antibodies and screens for modulators of headpin.

Headpin is a differentially expressed, novel serine proteinase inhibitor that belongs to the ov-serpin family and demonstrates a hinge region consensus sequence that predicts an inhibitory function. Headpin was cloned from a keratinocyte cDNA library, and its expression pattern by Northern blot analysis indicates that it is most likely produced by keratinizing epithelium. The endogenous expression of headpin in normal oral keratinocytes, and its absence or downregulation in squamous cell carcinoma of the oral cavity, supports the involvement of headpin as a marker for squamous differentiation or a gene disadvantageous to tumor function. Headpin has been grouped into the cluster of serpins located at chromosome 18q21.3/18q22. This region is a known area for LOH and other deletional events often associated with head and neck cancer.

The invention first provides an isolated polynucleotide encoding a polypeptide having an amino acid sequence of SEQ ID NO:2. In certain embodiments, the polynucleotide comprises a nucleic acid sequence of SEQ ID NO:1 or a complement thereof. In other embodiments, the polynucleotide further comprises a promoter operable in eukaryotic cells. In certain aspects, the promoter is heterologous to the coding sequence.

The invention also provides a nucleic acid segment characterized as comprising a sequence region of at least about 6, about 7, about 8, about 9, about 10, about 11, about 12, about 13, about 14, about 15, about 16, about 17, about 18, about 19, about 20, about 21, about 22, about 23, about 24, about 25, about 25, about 26, about 27, about 28, about 29, about 30 about 30, about 31, about 32, about 33, about 34, about 35, about 36, about 37, about 38, about 39, about 40, about 41, about 42, about 43, about 44, about 45, about 46, about 47, about 48, about 49, or about 50 contiguous nucleotides, and any range derivable therein, that have the same sequence as, or are complementary to, about 6, about 7, about 8, about 9, about 10, about 11, about 12, about 13, about 14, about 15, about 16, about 17, about 18, about 19, about 20, about 21, about 22, about 23, about 24, about 25, about 25, about 26, about 27, about 28, about 29, about 30 about 30, about 31, about 32, about 33, about 34, about 35, about 36, about 37, about 38, about 39, about 40, about 41, about 42, about 43, about 44, about 45, about 46, about 47, about 48, about 49, or about 50 contiguous nucleotides of position 1 to 700 and position 801 to 1279 of SEQ ID NO:1, and any range derivable therein, and any integer derivable therein such a range, or a nucleic acid segment of from about 6, about 7, about 8, about 9, about 10, about 11, about 12, about 13, about 14, about 15, about 16, about 17, about 18, about 19, about 20, about 21, about 22, about 23, about 24, about 25, about 25, about 26, about 27, about 28, about 29, about 30 about 30, about 31, about 32, about 33, about 34, about 35, about 36, about 37, about 38, about 39, about 40, about 41, about 42, about 43, about 44, about 45, about 46, about 47, about 48, about 49, about 50 to about 1,000,000 nucleotides or more in length, and any range derivable therein, and any integer derivable therein such a range, that hybridizes to the nucleic acid segment, or the complement thereof, of position 1 to 700 and position 801 to 1279 of SEQ ID NO:1 under standard hybridization conditions, or preferably, under high stringency hybridization conditions.

As used herein, “any range derivable therein” means a range selected from the numbers described in the specification, and “any integer derivable therein” means any integer between such a range.

In addition to the hybridization conditions described herein in detail below, other “standard” and “high” stringency hybridization conditions are well known to those of skill in the art. An exemplary, but not limiting, standard hybridization is incubated at 42° C. in 50% formamide solution that may additionally contain 5×SSC or 5×SSPE, 0.5% to 2% SDS, 5× to 10×Denhardt's solution, 50 μg/ml to 100 μg/ml salmon sperm DNA or dextran sulfate for 24 to 48 hours and subjected to a final wash in 0.5×SSC, 0.1% SDS at 45° C. to 65° C. “High” stringency hybridization conditions are exemplified by, but not limited to, incubation at 42° C. in 50% formamide solution that may additionally contain 5×SSC or 5×SSPE, 0.5% to 2% SDS, 5× to 10×Denhardt's solution, 50 μg/ml to 100 μg/ml salmon sperm DNA or dextran sulfate for 24 to 48 hours and subjected to a final wash in 0.1×SSC, 0.1% SDS at 55° C. to 65° C. In addition to hybridization to Southern or northern blots, hybridization of primers for use in PCR™ is another preferred method for identification of sequences contemplated for use in the present invention.

Segments of SEQ ID NO:1 or the complements thereof, or the mutants thereof, may variously be about 30, about 35, about 40, about 45, about 50, about 60, about 70, about 80, about 90, about 100, about 110, about 120, about 130, about 140, about 150, about 160, about 170, about 180, about 190, about 200, about 210, about 220, about 230, about 240, about 250, about 300, about 350, about 400, about 450, about 500, about 600, about 650, about 700, about 750, about 800, about 850, about 900, about 950, about 1000, about 1050, about 1100, about 1150, about 1200 or so nucleotides in length, up to and including the full length sequence of 1279 contiguous nucleotides of SEQ ID NO: 1, or even longer, as well as any range derivable therein and any integer derivable therein such a range, as may be achieved by duplication of certain domains.

Any segment may be combined into a DNA segment or vector of up to or even longer than about 1,000,000, about 950,000, about 900,000, about 850,000, about 800,000, about 750,000, about 700,000, about 650,000, about 600,000, about 550,000, about 500,000, about 450,000, about 400,000, about 350,000, about 300,000, about 250,000, about 200,000, about 150,000, about 100,000, about 75,000, about 50,000, about 40,00, about 30,000, about 20,000, about 15,000, about 10,000, about 5,000, about 3,000, about 2,000, to about 1,000 basepairs in length are also provided, as well as any range derivable therein and any integer derivable therein such a range. Segments of up to about 20,000, about 15,000, about 10,000, about 5,000 and about 3,000 basepairs in length, and any range derivable therein and any integer derivable therein such a range are preferred. The nucleic acids of the present invention may also be DNA segments, peptide nucleic acid segments, or RNA segments.

The invention provides an isolated and purified nucleic acid that hybridizes, under high stringency conditions, to a DNA segment comprising about 102 to 1279 bases of SEQ ID NO:1, or a complement thereof.

The invention provides a nucleic acid of about 15 to about 5000 base pairs comprising at least 102 contiguous base pairs of SEQ ID NO:1, or the complement thereof.

The invention provides an isolated and purified nucleic acid that hybridizes, under high stringency conditions, to a DNA segment comprising about 15 to 700 bases of SEQ ID NO:18, or a complement thereof.

The invention provides an isolated and purified nucleic acid that hybridizes, under high stringency conditions, to a DNA segment comprising about 15 to 478 bases of SEQ ID NO:20, or a complement thereof.

The invention provides a nucleic acid of about 15 to about 5000 base pairs, and any range derivable therein, and any integer derivable therein such a range, comprising about 15, about 20, about 30, about 50, about 100, about 150, about 250, or about 478, and any range derivable therein, and any integer derivable therein such a range, of SEQ ID NO:18 or SEQ ID NO:20, or the complement thereof.

The invention provides a nucleic acid of about 15 to about 5000 base pairs, and any range derivable therein, and any integer derivable therein such a range, comprising about 15, about 20, about 30, about 50, about 100, about 150, about 250, about 478, about 550, to about 700 contiguous base pairs, and any range derivable therein, and any integer derivable therein such a range, of SEQ ID NO:18, or the complement thereof.

The invention provides a DNA segment comprising an isolated polynucleotide that encodes a mammalian headpin protein, polypeptide, or peptide. In certain embodiments of the present invention, the isolated polynucleotide is a mammalian headpin isolated polynucleotide. In certain aspects, the mammalian isolated polynucleotide is a human or mouse. In other embodiments, the isolated polynucleotide is a gene.

The invention provides a peptide comprising at least 34 contiguous amino acids of SEQ ID NO:2, or a biological functional equivalent thereof.

The invention provides a polypeptide comprising at least 51 contiguous amino acids of SEQ ID NO:2, or a biological functional equivalent thereof.

The invention provides a peptide comprising about 10, about 15, about 20, about 25, about 30, or about 50 contiguous amino acids, and any range derivable therein, and any integer derivable therein such a range, of SEQ ID NO:19 or SEQ ID NO:21, or a biological functional equivalent thereof.

As used herein in the context of various of the instant compositions and methods, a “proteinaceous molecule”, “proteinaceous composition”, “proteinaceous compound”, “proteinaceous chain” or “proteinaceous material” generally refers, but is not limited to, a protein, polypeptide or peptide. All the “proteinaceous” terms described above may be used interchangably herein. As used herein, a “protein” will be understood to mean a amino acid chain that is longer than about 100 contiguous amino acids and in most aspects comprises more that about 70% of the amino acids encoded by a gene; a polypeptide of greater than about 50 amino acids; and/or a peptide of from about 3 to about 50 amino acids in length. Thus, mammalian headpin proteinaceous segments of varying overall length that retain catalytic, regulatory and structural properties and functions are provided herein.

As used herein, in the context of various of the instant compositions and methods, the term “mammalian headpin protein” or the term “headpin protein” will be understood to include wild-type, polymorphic and mutant versions of mammalian headpin protein sequences. The invention thus also includes the provision of DNA segments, vectors, genes and coding sequence regions that encode various forms of mammalian headpin proteins, polypeptides, domains, peptides or any fusion protein, polypeptide, or peptide thereof.

Additionally, the invention provides mammalian headpin protein, polypeptide or peptide elements that comprise at least one mutation in comparison to the wild-type sequence. The mutation may be deliberately introduced by the hand of man, for example, in order to test the function of the changed amino acid, e.g., the interaction of a mammalian headpin protein, polypeptide or peptide with other proteins, polypeptides, peptides, or nucleic acids and/or other functions. Additionally, the mutation may be a naturally occurring polymorphic change, either isolated from normal cells or introduced by the hand of man. The mammalian headpin mutation may also be in a purified protein, polypeptide, or peptide obtained directly from an aberrant cell, such as a tissue or secretion from a patient having a disease characterized by aberrant headpin activity, or may be a recombinant protein, polypeptide, or peptide that has been changed to introduce a mutation that mirrors one identified in a patient. The mutation may result in a mammalian headpin protein gene or protein, polypeptide, or peptide of altered length, or may result in increased, decreased or undetectable levels of a mammalian headpin gene message or protein, polypeptide, or peptide being produced, and/or may result in increased, decreased or undetectable levels of a another mammalian gene message or protein, polypeptide, or peptide coexpressed in the same cell.

The protein compositions provided by the invention may thus be further characterized as including an isolated headpin protein, polypeptide, or peptide that comprises a contiguous amino acid sequence of at least about 13 amino acids from SEQ ID NO:2, or biologically functional equivalents thereof. In preferred embodiments, the headpin protein comprises an isolated protein of about 391 amino acids in length, comprising the amino acid sequence of SEQ ID NO:2, or biologically functional equivalents thereof.

The headpin fusion proteins or constructs including headpin sequences operatively attached to distinct, selected or designed amino acid sequences, such as selected or designed antigenic amino acid sequences, amino acid sequences with selected or designed binding affinity for a selected or designed molecule, and DNA binding or transactivation amino acid sequences, also are encompassed within the invention. Fusion proteins with selectably-cleavable bonds or linkers, such as biologically-cleavable, enzymatically-cleavable and chemically-cleavable bonds or linkers, are also provided.

The headpin proteinaceous compositions will include the same types of isoforms and mutants as described above for the nucleic acids. The use of specific isoforms or mutated headpin proteins, polypeptides, and/or peptides to prepare isoform- or mutant-specific antibodies is particularly contemplated. In terms of diagnostic isoform or mutated headpin peptides and antibodies, these compositions will generally be more useful in regard to small deletions or point mutants, whereas nucleic acid probes may be more suitable for detecting larger deletions, or duplication, translocation and insertional mutations in addition to point mutants.

In certain embodiments of the present invention, the isolated polynucleotide that encodes headpin protein, polypeptide, or peptide comprises a contiguous amino acid sequence of at least about 8 amino acids from between position 1 to 224 and from position 257 to 391 of SEQ ID NO:2, or biologically functional equivalents thereof. As used herein in various aspects of the invention, the term “contiguous amino acid sequence” will be understood to include a contiguous amino acid sequence of at least about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150 about 151, about 152, about 153, about 154, about 155, about 156, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 1.87, about 188, about 189, about 190, about 195, about 200, about 210, about 220, about 230, about 240, about 250, about 260, about 270, about 280, about 290, about 300, about 310, about 320, about 330, about 340, about 340, about 350, about 360, about 370, about 380, or about 390 amino acids or so, and any range derivable therein, and any integer derivable therein such a range.

In further embodiments of the present invention, the isolated headpin polynucleotide includes a contiguous nucleic acid sequence of at least about 24 nucleotides from between position 1 to 700 and between position 801 to 1279 of SEQ ID NO:1, or the complement thereof thereof.

As used herein in particular embodiments of the invention, the term “contiguous nucleic acid segment” will be understood to include a contiguous nucleic acid sequence of about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95, about 100, about 105, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400 about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 720, about 730, about 740, about 750, about 750, about 800, about 850, about 900, about 950, about 1000, about 1050, about 1100, about 1150, about 1200, to about 1279 nucleotides or so, and any range derivable therein, and any integer derivable therein such a range.

In particular aspects of the present invention the isolated polynucleotide encodes a headpin protein of about 391 amino acids in length that comprises the amino acid sequence of SEQ ID NO:2 or biologically functional equivalents thereof. In a preferred aspect, the DNA segment comprises an isolated headpin polynucleotide that includes the contiguous nucleic acid sequence from between position 1 and position 1279 of SEQ ID NO:1, or a complement thereof.

The DNA segments and coding regions may encode wild-type or mutant headpin peptides, e.g., of about 6, about 7, about 8, about 9, about 10, about 11, about 12, about 13, about 14, about 15, about 16, about 17, about 18, about 19, about 20, about 21, about 22, about 23, about 24, about 25, about 25, about 26, about 27, about 28, about 29, about 30 about 30, about 31, about 32, about 33, about 34, about 35, about 36, about 37, about 38, about 39, about 40, about 41, about 42, about 43, about 44, about 45, about 46, about 47, about 48, about 49, or about 50 amino acids in length or so, and any range derivable therein and any integer derivable therein such a range. The headpin peptides may be lacking in any defined headpin activity, and may, for example, be used in generating antibodies or in other embodiments. The headpin peptides or domains may also be deliberately engineered to include a mutation, e.g., in order to prepare antibodies that are specific for a mutated headpin, particularly where the mutation represents one identified in a patient with cancer or other disease. The present invention also provides DNA segments and coding regions that may encode headpin peptide of about 6 to about 30 amino acids in length and any integer derivable therein, the peptide having an amino acid sequence that corresponds to a wild-type headpin sequence of a headpin protein, polypeptide, or peptide sequence region that is susceptible to mutations that are indicative of a disease phenotype.

The isolated polynucleotides, genes, nucleic acid segments, vectors and coding sequence regions may also encode headpin proteins, polypeptides, or peptides with certain, but not necessary all, headpin functional properties. As such, isolated polynucleotides, genes, nucleic acid seqments and coding sequences encoding isolated mutant headpin domains are provided. The headpin domains may be mutant domains, which include naturally occurring polymorphisms and mutations found in headpin proteins, polypeptide, or peptides in patients and, also, mutations deliberately engineered into a domain to test their function in assays. The mutant domains are also useful in antibody generation and in various in vitro and cellular assays.

Isolated polynucleotides, nucleic acid segments, isolated genes or coding regions may be also manipulated to encode headpin fusion proteins or constructs in which at least one headpin protein, polypeptide, or peptide sequence is operatively attached or linked to at least one distinct, selected amino acid sequence. The combination of headpin sequences with selected antigenic amino acid sequences; selected non-antigenic carrier amino acid sequences, for use in immunization; selected adjuvant sequences; amino acid sequences with specific binding affinity for a selected molecule; and amino acid sequences that form an active DNA binding or transactivation domain are particularly contemplated. Certain fusion proteins may be linked together via a protease-sensitive peptide linker, allowing subsequent easy separation. Isolated polynucleotides, nucleic acid segments, coding regions and isolated genes may also be manipulated to encode headpin fusion proteins or constructs in which at least one headpin protein, polypeptide, or peptide sequence is operatively attached or linked to at least one distinct, selected headpin protein, polypeptide, or peptide sequence.

Also contemplated are isolated polynucleotides and nucleic acid segments that comprise at least a first coding region that encodes a headpin protein, polypeptide, or peptide and at least a second coding region that encodes a second, distinct selected protein, polypeptide, or peptide. This aspect of the invention is termed coexpression, which is distinct from expression of a fusion protein, polypeptide, or peptide.

The invention provides an expression cassette comprising a polynucleotide or nucleic acid segment encoding a polypeptide having the sequence of SEQ ID NO:2, SEQ ID NO:19 or SEQ ID NO:21, or the biological functional equivalent thereof, wherein the polynucleotide or nucleic acid segment is under the control of a promoter operable in eukaryotic cells.

The isolated polynucleotides or DNA segments intended for use in expression will be operatively positioned under the control of, i.e., downstream from, a promoter that directs expression of headpin in a desired host cell, such as E. coli, or in certain preferred embodiments in a mammalian or human cell. The promoter may be a recombinant promoter or a promoter naturally associated with a headpin gene. Recombinant vectors thus form another aspect of the present invention. The use of isolated headpin genes positioned, in reverse orientation, under the control of a promoter that directs the expression of an antisense product in a cell is also contemplated.

The invention also provides a cell comprising an expression cassette comprising a polynucleotide or nucleic acid segment encoding a polypeptide having the sequence of SEQ ID NO:2, SEQ ID NO:19 or SEQ ID NO:21, or the biological functional equivalent thereof, wherein the polynucleotide or nucleic acid segment is under the control of a promoter operable in eukaryotic cells. The present invention further provides recombinant host cells comprising at least one isolated polynucleotide or isolated nucleic acid segment segment or vector that encodes a headpin protein, polypeptide, domain, peptide, or any fusion protein or mutant thereof. In certain embodiments the isolated polynucleotide or isolated nucleic acid segement comprises and isolated gene, a headpin protein, polypeptide, domain, peptide, or any fusion protein or mutant thereof. In certain preferred embodiments of the present invention the recombinant host cell comprises an isolated headpin polynucleotide that includes the contiguous nucleic acid sequence from between position 1 and position 1279 of SEQ ID NO:1, or the complement thereof. Prokaryotic recombinant host cells, such as E. coli, are provided, as are eukaryotic host cells, including mammalian, or preferably yeast (Saccharomyces cerevieae) or a baculovirus cells provided with the headpin constructs of the invention. In preferred embodiments, the proteins, polypeptides, peptides, or fusion proteins expressed will have native activity, although the level of activity may vary from system to system.

The recombinant host cells of the present invention preferably have one or more isolated polynucleotides or nucleic acid segments introduced into the cell by means of a recombinant vector, and preferably express the isolated polynucleotide or nucleic acid segment to produce the encoded headpin protein, polypeptide, or peptide. More preferred is where the cells express a headpin protein, polypeptide, or peptide that includes a contiguous amino acid sequence of at least about 13 amino acids from SEQ ID NO:2, or a biological functional equivalent thereof, and still more preferred is where the cells express a headpin protein, polypeptide, or peptide that includes a contiguous amino acid sequence of at least about 391 amino acids from SEQ ID NO:2, or a biological functional equivalent thereof.

Thus, the present invention provides methods of using headpin isolated polynucleotides or nucleic acid segments that comprise expressing a headpin isolated polynucleotide or nucleic acid segment in a recombinant host cell and collecting the headpin protein, polypeptide, peptide, domain or mutant expressed by said cell. These methods may be characterized as represented by the steps of preparing a recombinant vector in which a headpin-encoding isolated polynucleotide or nucleic acid segment is positioned under the control of a promoter, introducing the recombinant vector into a recombinant host cell, culturing the recombinant host cell under conditions effective to allow expression of an encoded headpin protein, polypeptide, peptide, domain, or mutant, and collecting the expressed headpin protein, polypeptide, peptide, domain, or mutant.

In further embodiments, the present invention provides headpin proteins, polypeptides, domains, peptides, mutants and any fusion proteins thereof prepared by recombinant means. Recombinant headpin proteins, polypeptides, and peptides may be defined as being prepared by expressing a headpin protein, polypeptide, or peptide in a recombinant host cell and purifying the expressed headpin protein, polypeptide, or peptide away from total recombinant host cell components. The headpin protein compositions will generally be obtained free from total cell components, and will comprise at least one type of isolated headpin protein, polypeptide, or peptide, purified relative to the natural level in a given cell.

The invention also provides a method for treating a subject with cancer comprising the step of administering to the subject a nucleic acid (i) encoding a headpin protein, polypeptide or peptide and (ii) a promoter active in eukaryotic cells, wherein the promoter is operably linked to the region encoding the headpin protein, polypeptide or peptide. In certain aspects, the promoter is an inducible promoter.

In certain embodiments, the promoter is heterologous to the coding sequence. In other embodiments, the promoter is an inducible promoter. In some embodiments, the expression cassette is contained in a viral vector. In certain aspects, the viral vector is selected from the group consisting of a retroviral vector, an adenoviral vector, and adeno-associated viral vector, a vaccinia viral vector, and a herpesviral vector. In other aspects, the expression cassette further comprises a polyadenylation signal. In particular aspects, the expression cassette comprises a second polynucleotide encoding a second polypeptide. In certain facets, the second polynucleotide is under the control of a second promoter. In other facets, the expression cassette of claim 27, wherein the polynucleotide is positioned, in reverse orientation, under the control of a promoter that directs expression of an antisense product.

The invention provides a hybridoma cell that produces a monoclonal antibody that binds immunologically to a polypeptide having the sequence of SEQ ID NO:2, SEQ ID NO:19 or SEQ ID NO:21, or an immunologic fragment thereof.

Further compositions of the present invention are antibodies, monoclonal antibodies and antibody conjugates, that have immunospecificity for a headpin protein, polypeptide, or peptide. The antibodies may be operatively attached to a detectable label. The antibodies and antibody conjugates may be specific for the headpin isoform, or mutant headpin proteins, polypeptides, or peptides and allow differential binding from, in the case of headpin, wild-type headpin.

The invention provides a monoclonal antibody that binds immunologically to a polypeptide having the sequence of SEQ ID NO:2, SEQ ID NO:19 or SEQ ID NO:21, or an immunologic fragment thereof. In certain embodiments, the antibody further comprises a detectable label. In other embodiments, the label is selected from the group consisting of a fluorescent label, a chemiluminescent label, a radiolabel and an enzyme.

The invention also provides a polyclonal antisera, antibodies of which bind immunologically to a polypeptide having the sequence of SEQ ID NO:2, SEQ ID NO:19 or SEQ ID NO:21, or an immunologic fragment thereof.

Where diagnostic or prognostic isoform or mammalian headpin proteins, polypeptides or peptides, or genes or nucleic acids encoding headpin proteins, polypeptides or peptides are concerned (whether an isoform or mutant), the headpin protein, polypeptide or peptide, or encoded headpin protein, polypeptide or peptide by a gene or nucleic acid (e.g., DNA segment), will preferably have a different specificity for the mammalian headpin binding proteinaceous sequence, and more preferrably a greater binding specificity than the wild-type mammalian headpin proteinaceous sequence, allowing effective differentiation between them, as may be used in diagnostic or prognostic tests for cancer in a subject as described below.

The present invention also provides a method for detecting headpin in a sample, comprising contacting sample nucleic acids from a sample suspected of containing headpin with at least a first headpin nucleic acid segment, or a complement thereof, under conditions effective to allow hybridization of substantially complementary nucleic acids, and detecting the hybridized complementary nucleic acids thus formed. In certain preferred embodiments of the present invention, the first headpin nucleic acid segment encodes a headpin protein, polypeptide, or peptide, and the first nucleic acid segment, or complement thereof, is used to detect hybridizing complementary nucleic acids thus formed. The first nucleic acids may be DNA, RNA, or a peptide nucleic acid.

In particular embodiments, the detection of headpin will be in the context of cDNA or genomic cloning or gene manipulation. In certain preferred aspects, the sample nucleic acids are obtained from a clinical sample from a human suspected of having a cancer. In some embodiments, the sample nucleic acids contacted are located within a cell, while in alternative aspects, the sample nucleic acids are separated from a cell prior to contact. The sample nucleic acids may be DNA or RNA. In particular aspects of the present invention, the isolated headpin nucleic acid segment comprises a detectable label, for example a radio, enzymatic or fluorescent label, and the hybridized complementary nucleic acids are detected by detecting the label.

The present invention also provides a method for detecting headpin in a sample that may be further characterized as comprising the steps of contacting the sample nucleic acids with at least two nucleic acid primers that hybridize to distant sequences from a headpin sequence, the primers capable of amplifying a headpin nucleic acid segment when used in conjunction with a polymerase chain reaction, conducting a polymerase chain reaction to create amplification products, and detecting the amplification products thus formed. In certain exemplary embodiments, more than one primer pair, such as in quadriplex (two primer pairs) or multiplex (multiple primer pairs) amplification, may be used.

The invention also provides for a method of diagnosing cancer in a subject, involving comparing the amount of headpin activity, RNA or protein, polypeptide or peptide in a candidate cell to the amount of headpin activity, RNA or protein, polypeptide or peptide in a normal cell, wherein lower amounts of headpin activity, RNA or protein, polypeptide or peptide in a candidate cell is diagnostic for cancer.

The invention further provides a method of diagnosing a cancer comprising the steps of: (i) obtaining a tissue sample from a subject; and (ii) assessing the expression of a headpin nucleic acid or a headpin polypeptide in cells of the sample. In certain embodiments, the assessing comprises assaying for a headpin nucleic acid from the sample. In some embodiments, the method further comprises subjecting the sample to conditions suitable to amplify the nucleic acid. In other aspects, the assessing comprises contacting the sample with an antibody that binds immunologically to a headpin polypeptide or peptide. In some facets, the method further comprises subjecting proteins, polypeptides or peptides of the sample to ELISA. In other facets, the method further comprises the step of comparing the expression of a headpin nucleic acid, polypeptide or peptide with the expression of headpin in non-cancer samples. In some embodiments, the assessing involves evaluating the structure of the headpin gene or transcript.

The invention also provides a nucleic acid detection kit comprising, in suitable container means, at least a first isolated mammalian headpin nucleic acid segment and a detection reagent. Thus, the present invention also provides a nucleic acid detection kit that may comprise, in suitable container means, at least a first isolated headpin nucleic acid segment, or the complement thereof, and a detection reagent. The nucleic acid detection kit may further comprise an unrelated nucleic acid segment for use as a control, and/or a restriction enzyme. In certain embodiments, the nucleic acid detection kit may comprise at least two headpin nucleic acid segments of between about 14 and about 30 nucleotides in length. The first nucleic acids may be DNA, RNA, or a peptide nucleic acid. In particular aspects, the detection reagent may be a detectable label that is linked to the headpin nucleic acid segment.

The present invention also provides methods for detecting headpin in a sample, comprising contacting sample proteins, polypeptides, or peptides from a sample suspected of containing headpin with at least a first antibody that binds to a headpin protein, polypeptides, or peptide, under conditions effective to allow the formation of immune complexes, and detecting the immune complexes thus formed. In certain aspects, the sample proteins, polypeptides, or peptides contacted are located within a cell, while in others, the sample proteins, polypeptides, or peptides are separated from a cell prior to contact.

The invention provides an immunodetection kit comprising, in suitable container means, at least a first antibody that binds to a mammalian headpin protein, polypeptide, or peptide and a detection reagent. The present invention also provides an immunodetection kit that may comprise, in suitable container means, at least a first antibody capable of binding to a headpin protein, polypeptide, or peptide and a detection reagent. In certain aspects of the invention, the first antibody is a monoclonal antibody. In other aspects, the immunodetection kit may further comprise an unrelated protein, polypeptide, or peptide for use as a control, and/or a second antibody that binds to the first antibody.

The “type” of headpin may be determined, allowing the headpin mutants or alleles to be distinguished from the other headpin isoforms, or mutant genes and proteinaceous compositions to be distinguished from wild-types. The use of isoform-, mutant- and wild-type-specific nucleic acid probes is particularly contemplated. In the beginning, the use of wild-type-specific nucleic acid probes will be preferred. The identification of a particularly diagnostic isoform or mutant sequence will then lead to the increased use of that isoform or mutant sequence, either in the population or in defined families. The use of isoform-, mutant- and wild-type-specific antibodies is also contemplated, as may be prepared using isoform-, mutant- and wild-type-specific headpin peptides.

Where the “amount” or “activity” of headpin is determined, a lesser or greater amount of the natural headpin protein or protein activity may be indicative of the extent of disease pathogenesis or cancer. In any event, changes from the naturally observed presence or absence of headpin activity or protein in the population will be easily detected and will have implications for disease risk, development, and progression.

The type or amount of headpin may be determined by means of a molecular biological assay to determine the type or amount of a nucleic acid that encodes headpin. Such molecular biological assays will often comprise a direct or indirect step that allows a determination of the sequence of at least a portion of the headpin-encoding nucleic acid, which sequence can be compared to a wild-type headpin sequence, such as SEQ ID NO:1, a complement thereof, or another acceptable normal allelic or polymorphic sequence.

It is contemplated that headpin sequences diagnostic or prognostic for a particular disease may comprise at least one point mutation, deletion, translocation, insertion, duplication or other aberrant change. Diagnostic RFLPs are thus also contemplated. RNase protection assays may also be employed in certain embodiments.

Diagnostic methods may be based upon the steps of obtaining a biological sample from a subject or patient, contacting sample nucleic acids from the biological sample with a first isolated headpin nucleic acid segment, or the complement thereof, under conditions effective to allow hybridization of substantially complementary nucleic acids, and detecting, and optionally further characterizing, the hybridized complementary nucleic acids thus formed. The first isolated headpin nucleic acid segment may comprise a DNA, or RNA, or an peptide nucleic acid segment representing a portion or all of headpin.

The methods may involve in situ detection of sample nucleic acids located within the fluids of the sample derived from the patient. The sample nucleic acids may also be separated from the biological material prior to contact. The sample nucleic acids may be DNA or RNA. The methods may involve the use of first isolated headpin nucleic acid segments that comprise a radioactive, enzymatic or fluorescent detectable label, wherein the hybridized complementary nucleic acids are detected by detecting the label.

PCR™ will often be preferred, as may be exemplified by the steps of contacting the sample nucleic acids with a pair of nucleic acid primers that hybridize to distant sequences from a mutant, polymorphic or wild-type headpin nucleic acid sequence, the primers capable of amplifying a mutant, polymorphic or wild-type headpin nucleic acid segment when used in conjunction with a polymerase chain reaction, conducting a polymerase chain reaction to create amplification products, and detecting and characterizing the amplification products thus formed.

Diagnostic immunoassay methods are also provided, wherein the type or amount of mammalian headpin is determined by means of an immunoassay to determine the type or amount of a headpin protein, polypeptide, or peptide. Such methods may comprise the steps of obtaining a biological sample from a subject or patient, contacting the biological sample with a first antibody that binds to a headpin protein, polypeptide, or peptide, or mutant, under conditions effective to allow the formation of specific immune complexes, and detecting the specific immune complexes thus formed. The first antibody may be of monoclonal or polyclonal origin.

The first antibody may be linked to a detectable label, wherein the immune complexes are directly detected by detecting the presence of the label. The immune complexes may also be indirectly detected by means of a second antibody linked to a detectable label, the second antibody having binding affinity for the first antibody.

The activity of headpin may be determined by means of a biological assay, to determine the activity of a headpin protein, polypeptide, or peptide. Methods for determining the activity of headpin may comprise the steps of obtaining a biological sample from a subject or patient, contacting the biological sample with a substrate such as a serine protease, under conditions effective to allow the association of headpin to the substrate, and detecting the amount of bound headpin.

In still further embodiments, the present invention concerns a method for identifying new compounds that alter headpin binding activity, which may be termed as “candidate substances.” It is contemplated that this screening technique will prove useful in the general identification of any compound that will serve the purpose of altering headpin binding activity.

It is further contemplated that useful compounds in this regard will in no way be limited to proteinaceous or peptidyl compounds or oligonucleotides. In fact, it may prove to be the case that the most useful pharmacological compounds for identification through application of the screening assay will be non-peptidyl in nature and serve to alter the headpin binding activity through a tight binding or other chemical interaction.

Accordingly, in screening assays to identify pharmaceutical agents which disrupt or enhance headpin activity, it is proposed that compounds isolated from natural sources, such as animals, bacteria, fungi, plant sources, including leaves and bark, and marine samples may be assayed for the presence of potentially useful pharmaceutical agents. It will be understood that the pharmaceutical agents to be screened could also be derived from chemical compositions or man-made compounds.

In important aspects, the candidate substances may be chemical compounds or anti-headpin antibodies, including polyclonal and monoclonal antibodies. The suspected agents could also include proteins, polypeptides, and peptides, such as those derived from recombinant DNA technology or by other means, including peptide synthesis. The active compounds may include fragments or parts of naturally-occurring compounds or may be only found as active combinations of known compounds which are otherwise inactive.

The invention also provides a method for identifying a candidate substance that modulates mammalian headpin activity, comprising the steps of: (a) admixing a mammalian headpin composition that comprises a candidate substance, a serine protease, and a mammalian headpin protein, polypeptide or peptide; and (b) determining the ability of the candidate substance to modulate the ability of the mammalian headpin protein, polypeptide or peptide to inhibit the serine protease.

Particular aspects of the present invention are directed to methods for identifying a candidate substance that modulates or alters headpin activity, which may be characterized as comprising the steps of admixing a composition that comprises a candidate substance, a serine protease, and a headpin protein, polypeptide, or peptide, and determining the ability of the headpin protein, polypeptide, or peptide to interact with the serine protease in the presence of the candidate substance and in the absence of the candidate substance, wherein the ability of a candidate substance to decrease the interaction of a headpin protein, polypeptide, or peptide to the serine protease is indicative of a candidate substance that modulates headpin activity.

In further embodiments, the present invention is directed to a method for identifying a candidate substance that stimulates headpin activity, an agonist, which may be characterized as comprising the steps of admixing a composition that comprises a candidate substance, a serine protease, and a headpin protein, polypeptide, or peptide, and determining the ability of the headpin protein, polypeptide, or peptide to interact with the serine protease in the presence of the candidate substance and in the absence of the candidate substance, wherein the ability of a candidate substance to increase the interaction of a headpin protein, polypeptide, or peptide to the serine protease is indicative of a candidate substance that modulates headpin activity.

In other embodiments, the present invention is directed to a method for identifying a candidate substance that inhibits headpin activity, an antagonist, which may be characterized as comprising the steps of admixing a composition that comprises a candidate substance, a serine protease, and a headpin protein, polypeptide, or peptide, and determining the ability of the headpin protein, polypeptide, or peptide to interact with the serine protease in the presence of the candidate substance and in the absence of the candidate substance, wherein the ability of a candidate substance to decrease the interaction of a headpin protein, polypeptide, or peptide to the serine protease is indicative of a candidate substance that modulates headpin activity.

In certain embodiments of the present invention, the headpin protein, polypeptide, or peptide is prepared by recombinant means. Thus, an important aspect of the candidate substance screening assay herein is the ability to prepare a native or recombinant headpin composition in a relative purified form, for example, in a manner as discussed above. This is an important aspect of the candidate substance screening assay in that without at least a relatively purified preparation, one will not be able to assay specifically for activity modulation, alteration, inhibition or activation, as opposed to the effects of other substances in the extract which then might affect headpin. In any event, the successful isolation of headpin protein, peptide or polypeptide now allows for the first time the ability to identify and isolate new compounds which can be used for modulating, altering, inhibiting, or stimulating headpin activity.

The candidate screening assays are straightforward to set up and perform, and is related in many ways to the assay discussed above for determining headpin activity. Thus, after obtaining a relatively purified preparation of the headpin, either from native or recombinant sources, one will desire to simply admix a candidate substance with the headpin preparation, preferably under conditions which would allow the headpin to perform its function but for inclusion of an activity modulating, altering, inhibiting or stimulating substance.

An in vitro setting provides for a means of defining those dosage ranges that are effective in modulating, altering, reducing or stimulating headpin activity. As used herein, the term “effective amount” is used to describe the dosage of an agent identified as an inhibitor, modulator, or stimulator of headpin used to reduce or increase the activity to any amount below or above control levels. Preferably the reduction or increase will be of between about 10% to 50%, with 50% to 80% being preferred, and between about 80% to 100% being more preferred. It is contemplated that in certain instances of the invention the reduction or increase in the binding dissociation constant may increase or decrease by about 0.1, about 0.2, about 0.3, about 0.4, about 0.5, about 0.6, about 0.7, about 0.8, about 0.9, about 1, about 1.5, about 2, about 2.5, about 3, about 3.5, about 4, about 4.5, about 5, about 5.5, or about 6 orders of magnitude, and any range derivable therein.

It will, of course, be understood that all the screening methods of the present invention are useful in themselves notwithstanding the fact that effective candidates may not be found, since it would be a practical utility to know that headpin agonists or antagonists do not exist. The invention provides methods for screening for such candidates.

The invention provides a headpin modulator that may be prepared by a process comprising the steps of, admixing an enzyme composition that comprises a candidate modulator, a serine protease, and a headpin protein, polypeptide, or peptide, and determining the ability of the headpin protein, polypeptide, or peptide to interact with the serine protease in the presence of the candidate substance and in the absence of the candidate substance, wherein the ability of a candidate substance to modulate the interaction of a headpin protein, polypeptide, or peptide to the serine protease is indicative of a candidate substance that increases or decreases headpin activity, and obtaining the headpin activity stimulator so identified.

The invention also provides a headpin protein, polypeptide, or peptide modulator that may be prepared by a process comprising the steps of, admixing a composition that comprises an RBP protein, polypeptide, or peptide, a serine protease, and a candidate modulator, identifying a modulator that alters headpin activity by determining the ability of the headpin protein, polypeptide, or peptide to interact with the serine protease in the presence of the candidate modulator and in the absence of the candidate modulator, wherein the ability of a candidate modulator to alter the binding of the headpin protein, polypeptide, or peptide to the serine protease is indicative of a modulator that alters headpin activity, and obtaining the modulator so identified. In certain aspects, the modulator stimulates activity (agonist) while in other aspects the modulator inhibits activity (antagonist). This is, of course, an important aspect of the invention in that it is believed that by inhibiting or stimulating headpin activity, one will be enabled to treat various aspects of diseases associated with headpin.

In certain aspects of the method, the headpin protein, peptide or polypeptide is comprised within a cell, preferably within or in contact with an animal. In more preferred aspects of the invention, the animal is a human.

The invention also provides a method of identifying a headpin substrate, comprising the steps of, obtaining an enzyme composition that comprises a headpin proteinaceous composition, admixing the headpin composition with a candidate substrate, and determining the ability of the headpin composition to bind the candidate substrate, wherein the ability to bind candidate substrate is indicative of a substrate that is a headpin substrate. In particular aspects, the substrate is a proteinaceous material.

Additionally, the invention provides a method of identifying a headpin protein, polypeptide, or peptide substrate, comprising the steps of, obtaining a composition that comprises a headpin protein, polypeptide, or peptide, admixing the protein, polypeptide, or peptide composition with a candidate substrate, and determining the ability of the protein, polypeptide, or peptide composition to bind a substrate, wherein the ability to bind the substrate is indicative of a candidate that is a headpin protein, polypeptide, or peptide substrate. In preferred aspects, the headpin substrate is a substrate that exists naturally in a mammalian cell.

The headpin proteins, polypeptides, domains, peptides and fusion proteins, the headpin nucleic acid segments, vectors, isolated genes and coding sequences, as well as the headpin modulators, inhibitors, stimulators, activity altering substances, or substrates may also be formulated with a pharmaceutically acceptable diluent or vehicle to form a headpin pharmaceutical composition in accordance with this invention. In particular embodiments of the present invention, the pharmaceutical composition comprises a recombinant vector that expresses a headpin protein, polypeptide, or peptide, or a headpin modulator, inhibitor, stimulator, activity altering substance, or substrate in a mammalian cell. In particular embodiments, the recombinant vector is incorporated into a virus, such as an adenovirus, adeno-associated virus, retrovirus, herpesvirus, or other recombinant virus. Such viruses can be formulated into a pharmaceutical composition.

Where a decrease in the amount or activity of headpin proves to be diagnostic of diseases such as certain types of cancers, the present invention also provides methods of treating cancers, comprising administering to a patient with a cancer a therapeutically effective amount of a pharmaceutically acceptable headpin composition. These treatments may also comprise giving headpin protein, polypeptide, or peptide compositions, headpin polynucleotides, nucleic acid segments or recombinant vectors that express headpin proteins, polypeptides, or peptides in the target cells, or headpin modulator, stimulator, or activity altering compositions. Enhancing headpin transcription, translation, stability or activity is also contemplated.

Alternatively, where an increase in the amount or activity of headpin proves to be diagnostic of diseases such as certain types of cancers, the invention further provides methods of treating cancers, comprising administering to a patient with cancers a therapeutically effective amount of a pharmaceutically acceptable composition that inhibits, modulates, or alters the activity of headpin. The composition may comprises a component that inhibits a headpin gene, mRNA, protein, polypeptide, peptide or enzyme activity. Examples of inhibitors include antisense constructs, ribozymes, inhibitory antibodies, and recombinant vectors that express any of the foregoing headpin inhibitors in mammalian cells.

The invention further provides a method of identifying a mammalian nucleic acid segment that comprises an isolated coding region or gene that encodes a mammalian headpin protein, polypeptide, or peptide, that may be characterized as comprising the steps of, obtaining at least one isolated nucleic acid segment designed to hybridize to a mammalian headpin gene, contacting a population of mammalian nucleic acids with the isolated nucleic acid segment under conditions effective to allow hybridization of the isolated nucleic acid segment to mammalian headpin nucleic acids within the population of mammalian nucleic acids, and identifying a mammalian nucleic acid segment that hybridizes to the isolated nucleic acid segment. In certain aspects of the invention, the at least one isolated nucleic acid segment comprises a contiguous nucleic acid sequences from SEQ ID NO:1. In certain other embodiments the method comprises obtaining a pair of isolated nucleic acid segments designed to hybridize to spatially distant sequences from a mammalian headpin coding region or gene, and conducting a polymerase chain reaction under conditions effective to amplify the mammalian headpin coding region or gene located between the spatially distant sequences.

The invention also provides a method of identifying a mammalian nucleic acid segment that comprises an isolated coding region or gene that encodes a mammalian headpin protein, polypeptide, or peptide, that may be characterized as comprising, in any suitable order, the steps of, obtaining an antibody designed to bind to a mammalian headpin protein, polypeptide, or peptide, obtaining a mammalian nucleic acid segment operatively linked to a promoter capable of expressing a candidate protein, polypeptide, or peptide encoded by the candidate mammalian nucleic acid segment in a host cell, providing the mammalian nucleic acid segment to the host cell, the host cell comprising the candidate protein, polypeptide, or peptide, contacting the candidate protein, polypeptide, or peptide expressed by the mammalian nucleic acid segment in the host cell with the antibody under conditions effective to allow binding of the antibody to the candidate protein, polypeptide, or peptide, and detecting the binding of the antibody to the candidate protein, polypeptide, or peptide, thereby identifying the candidate mammalian nucleic acid segment as a mammalian nucleic acid segment that comprises a coding region that encodes a mammalian headpin protein, polypeptide, or peptide.

The invention provides a method for treating subject with cancer comprising the step of administering to the subject a headpin proteinaceous composition, or a biological functional equivalent thereof.

In specific embodiments, the cancer is selected from the group consisting of head and neck, brain, lung, liver, spleen, kidney, lymph node, small intestine, pancreas, blood cells, colon, stomach, breast, endometrium, prostate, testicle, ovary, skin, esophagus, bone marrow and blood cancer. In particular aspects, the cancer is head and neck cancer. In other aspects, the subject is a human.

The present invention also provides for a method of treating cancer in a subject by increasing the amount or activity of headpin in a cell by administering to a subject a pharmaceutical composition comprising, in a pharmaceutically acceptable excipient, an isolated mammalian headpin protein, polypeptide, or peptide, a mammalian headpin activity modulator or a recombinant vector that expresses a mammalian headpin protein, polypeptide, or peptide, or a mammalian headpin activity modulator in a mammalian cell. In particular embodiments, the recombinant vector is incorporated into a virus, such as an adenovirus, adeno-associated virus, retrovirus, herpesvirus, or other recombinant virus. Such viruses can be formulated into a pharmaceutical composition and administered to a patient.

Product an isolated polynucleotide encoding a polypeptide having an amino acid sequence of SEQ ID NO:2. Product an isolated polynucleotide encoding a polypeptide having an amino acid sequence of SEQ ID NO:2 for use as a medicament. Use of compound an isolated polynucleotide encoding a polypeptide having an amino acid sequence of SEQ ID NO:2 for the manufacture or a medicament for the treatment of disease: cancer such as, for example, head and neck cancer and/or cervical cancer.

Product an isolated and purified nucleic acid that hybridizes, under high stringency conditions, to a DNA segment comprising about 102 to 1279 bases of SEQ ID NO:1. Product an isolated and purified nucleic acid that hybridizes, under high stringency conditions, to a DNA segment comprising about 102 to 1279 bases of SEQ ID NO:1 for use as a medicament. Use of compound an isolated and purified nucleic acid that hybridizes, under high stringency conditions, to a DNA segment comprising about 102 to 1279 bases of SEQ ID NO:1 for the manufacture or a medicament for the treatment of disease: cancer such as, for example, head and neck cancer and/or cervical cancer.

Product a nucleic acid of about 15 to about 5000 base pairs comprising at least 102 contiguous base pairs of SEQ ID NO:1, or the complement thereof. Product a nucleic acid of about 15 to about 5000 base pairs comprising at least 102 contiguous base pairs of SEQ ID NO:1, or the complement thereof for use as a medicament. Use of compound a nucleic acid of about 15 to about 5000 base pairs comprising at least 102 contiguous base pairs of SEQ ID NO:1, or the complement thereof for the manufacture or a medicament for the treatment of disease: cancer such as, for example, head and neck cancer and/or cervical cancer.

Product an isolated and purified nucleic acid that hybridizes, under high stringency conditions, to a DNA segment comprising about 15 to 700 bases of SEQ ID NO:18. Product an isolated and purified nucleic acid that hybridizes, under high stringency conditions, to a DNA segment comprising about 15 to 700 bases of SEQ ID NO:18 for use as a medicament. Use of compound an isolated and purified nucleic acid that hybridizes, under high stringency conditions, to a DNA segment comprising about 15 to 700 bases of SEQ ID NO:18 for the manufacture or a medicament for the treatment of disease: cancer such as, for example, head and neck cancer and/or cervical cancer.

Product an isolated and purified nucleic acid that hybridizes, under high stringency conditions, to a DNA segment comprising about 15 to 478 bases of SEQ ID NO:20. Product an isolated and purified nucleic acid that hybridizes, under high stringency conditions, to a DNA segment comprising about 15 to 478 bases of SEQ ID NO:20 for use as a medicament. Use of compound an isolated and purified nucleic acid that hybridizes, under high stringency conditions, to a DNA segment comprising about 15 to 478 bases of SEQ ID NO:20 for the manufacture or a medicament for the treatment of disease: cancer such as, for example, head and neck cancer and/or cervical cancer.

Product a nucleic acid of about 15 to about 5000 base pairs comprising about 15 contiguous base pairs of SEQ ID NO:18 or SEQ ID NO:20, or the complement thereof. Product a nucleic acid of about 15 to about 5000 base pairs comprising about 15 contiguous base pairs of SEQ ID NO:18 or SEQ ID NO:20, or the complement thereof for use as a medicament. Use of compound a nucleic acid of about 15 to about 5000 base pairs comprising about 15 contiguous base pairs of SEQ ID NO:18 or SEQ ID NO:20, or the complement thereof for the manufacture or a medicament for the treatment of disease: cancer such as, for example, head and neck cancer and/or cervical cancer.

Product a peptide comprising at least 34 contiguous amino acids of SEQ ID NO:2. Product a peptide comprising at least 34 contiguous amino acids of SEQ ID NO:2 for use as a medicament. Use of compound a peptide comprising at least 34 contiguous amino acids of SEQ ID NO:2 for the manufacture or a medicament for the treatment of disease: cancer such as, for example, head and neck cancer and/or cervical cancer.

Product a polypeptide comprising at least 51 contiguous amino acids of SEQ ID NO:2. Product a polypeptide comprising at least 51 contiguous amino acids of SEQ ID NO:2 for use as a medicament. Use of compound a polypeptide comprising at least 51 contiguous amino acids of SEQ ID NO:2 for the manufacture or a medicament for the treatment of disease: cancer such as, for example, head and neck cancer and/or cervical cancer.

Product a peptide comprising about 10 contiguous amino acids of SEQ ID NO:19 or SEQ ID NO:21. Product a peptide comprising about 10 contiguous amino acids of SEQ ID NO:19 or SEQ ID NO:21 for use as a medicament. Use of compound a peptide comprising about 10 contiguous amino acids of SEQ ID NO:19 or SEQ ID NO:21 for the manufacture or a medicament for the treatment of disease: cancer such as, for example, head and neck cancer and/or cervical cancer.

Product an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells. Product an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells for use as a medicament. Use of compound an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells for the manufacture or a medicament for the treatment of disease: cancer such as, for example, head and neck cancer and/or cervical cancer.

Product a cell comprising an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells. Product a cell comprising an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells for use as a medicament. Use of compound a cell comprising an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells for the manufacture or a medicament for the treatment of disease: cancer such as, for example, head and neck cancer and/or cervical cancer.

Product a hybridoma cell that produces a monoclonal antibody that binds immunologically to a polypeptide having the sequence of SEQ ID NO:2, SEQ ID NO:19 or SEQ ID NO:21, or an immunologic fragment thereof. Product a hybridoma cell that produces a monoclonal antibody that binds immunologically to a polypeptide having the sequence of SEQ ID NO:2, SEQ ID NO:19 or SEQ ID NO:21, or an immunologic fragment thereof for use as a medicament. Use of compound a hybridoma cell that produces a monoclonal antibody that binds immunologically to a polypeptide having the sequence of SEQ ID NO:2, SEQ ID NO:19 or SEQ ID NO:21, or an immunologic fragment thereof for the manufacture or a medicament for the treatment of disease: cancer such as, for example, head and neck cancer and/or cervical cancer.

Product a monoclonal antibody that binds immunologically to a polypeptide having the sequence of SEQ ID NO:2, SEQ ID NO:19 or SEQ ID NO:21, or an immunologic fragment thereof. Product a monoclonal antibody that binds immunologically to a polypeptide having the sequence of SEQ ID NO:2, SEQ ID NO:19 or SEQ ID NO:21, or an immunologic fragment thereof for use as a medicament. Use of compound a monoclonal antibody that binds immunologically to a polypeptide having the sequence of SEQ ID NO:2, SEQ ID NO:19 or SEQ ID NO:21, or an immunologic fragment thereof for the manufacture or a medicament for the treatment of disease: cancer such as, for example, head and neck cancer and/or cervical cancer.

Product a polyclonal antisera, antibodies of which bind immunologically to a polypeptide having the sequence of SEQ ID NO:2, SEQ ID NO:19 or SEQ ID NO:21, or an immunologic fragment thereof. Product a polyclonal antisera, antibodies of which bind immunologically to a polypeptide having the sequence of SEQ ID .NO:2, SEQ ID NO:19 or SEQ ID NO:21, or an immunologic fragment thereof for use as a medicament. Use of compound a polyclonal antisera, antibodies of which bind immunologically to a polypeptide having the sequence of SEQ ID NO:2, SEQ ID NO:19 or SEQ ID NO:21, or an immunologic fragment thereof for the manufacture or a medicament for the treatment of disease: cancer such as, for example, head and neck cancer and/or cervical cancer.

Product a nucleic acid detection kit comprising, in suitable container means, at least a first isolated mammalian headpin nucleic acid segment and a detection reagent.

Product an immunodetection kit comprising, in suitable container means, at least a first antibody that binds to a mammalian headpin protein, polypeptide, or peptide and a detection reagent.

Product a nucleic acid (i) encoding a headpin protein, polypeptide or peptide and (ii) a promoter active in eukaryotic cells, wherein said promoter is operably linked to the region encoding said headpin protein, polypeptide or peptide. Product a nucleic acid (i) encoding a headpin protein, polypeptide or peptide and (ii) a promoter active in eukaryotic cells, wherein said promoter is operably linked to the region encoding said headpin protein, polypeptide or peptide for use as a medicament. Use of compound a nucleic acid (i) encoding a headpin protein, polypeptide or peptide and (ii) a promoter active in eukaryotic cells, wherein said promoter is operably linked to the region encoding said headpin protein, polypeptide or peptide for the manufacture or a medicament for the treatment of disease: cancer such as, for example, head and neck cancer and/or cervical cancer

Product a headpin proteinaceous composition. Product a headpin proteinaceous composition for use as a medicament. Use of compound a headpin proteinaceous composition for the manufacture or a medicament for the treatment of disease: cancer such as, for example, head and neck cancer and/or cervical cancer.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. cDNA sequence and deduced amino acids of headpin. The box indicates the hinge region of the reactive site loop. The position of the primer sets used for both the relative RT-PCR™ and synthesis of the probe for the Northern blots are indicated by arrows below the nucleotide sequence.

FIG. 2. Comparison of reactive site loop sequences in headpin (SEQ ID NO:3) with other ov-serpins. Consensus hinge region is identical to headpin. SCCA1: Squamous cell antigen 1 (SEQ ID NO:4), SCCA2: Squamous cell antigen 2 (SEQ ID NO:5), PAI2: Plasminogen activator inhibitor-2 (SEQ ID NO:6). The numbers of the amino acids correspond to the nomenclature of Schechter and Berger (Schechter and Berger, 1967). The location of the scissile bond and the conserved P1′ serine are indicated.

FIG. 3. RH mapping of human headpin. Headpin is located between WI-4461 and CHLC.GATA2E06 which locates at 18q21.3.

### DESCRIPTION OF ILLUSTRATIVE EMBODIMENTS

Diagnosis and therapy of tumors is a ongoing priority in clinical medicine. In particular, defects in the regulation of serine proteases and serine protease inhibitors have been implicated in cancer progression, migration and metastasis.

Once a change in one or more genes and the production of their encoded proteins are identified in tumors, diagnostic techniques may be used to detect tumors by these characteristic defects, and therapies can be designed to correct the defective gene or gene expression.

The present invention advances the field of cancer identification and treatment, by the identification a novel serine protease inhibitor, designated “headpin” as defective (e.g., underexpressed) in tumors. The present invention further provides isolated headpin nucleic acids and encoded proteins, polypeptides and peptides. The invention also provides diagnostics and therapeutics for use in diagnosing and treating cancer, including but not limited to head and neck cancer. These embodiments are described in detail herein below.

I. Headpin Serine Protease Inhibitor Nucleic Acids

In one embodiment, the present invention discloses a novel nucleic acid sequence and a novel protein encoded by the nucleic acid that has homology to the serine protease inhibitor or serpin family of genes and proteins.

A. Genes and DNA Segments

Important aspects of the present invention concern isolated DNA segments and recombinant vectors encoding headpin proteins, polypeptides or peptides, and the creation and use of recombinant host cells through the application of DNA technology, that express a wild-type, polymorphic or mutant head pin, using the sequence of SEQ ID NO:1, and biologically functional equivalents thereof.

The present invention concerns DNA segments, isolatable from mammalian cells, such as mouse or human cells, that are free from total genomic DNA and that are capable of expressing a protein, polypeptide or peptide that has headpin activity. As used herein, the term “DNA segment” refers to a DNA molecule that has been isolated free of total genomic DNA of a particular species. Therefore, a DNA segment encoding headpin refers to a DNA segment that contains wild-type, polymorphic or mutant headpin coding sequences yet is isolated away from, or purified free from, total mammalian genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified headpin gene refers to a DNA segment including headpin protein, polypeptide or peptide coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and engineered segments that express, or may be adapted to express, proteins, polypeptides, domains, peptides, fusion proteins and mutants of headpin encoded sequences.

“Isolated substantially away from other coding sequences” means that the gene of interest, in this case the headpin gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments and recombinant vectors incorporating DNA sequences that encode a headpin protein, polypeptide or peptide that includes within its amino acid sequence a contiguous amino acid sequence in accordance with, or essentially as set forth in, SEQ ID NO:2, corresponding to the headpin designated “human headpin”.

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferrably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2”, provided the biological activity of the protein is maintained. In particular embodiments, the biological activity of a headpin protein, polypeptide or peptide, or a biologically functional equivalent, comprises binding to one or more proteases, particularly serine proteases. In specific embodiments, the biological activity of a headpin protein, polypeptide or peptide, or a biologically functional equivalent, comprises inhibition of the activity of one or more proteases, particularly serine proteases, through binding. A preferred protease activity that may be inhibited by a headpin protein, polypeptide or peptide, or a biologically functional equivalent, is inhibition of the ability or rate of protealytic cleavage catalyzed by the protease.

In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:1. Again, DNA segments that encode proteins, polypeptide or peptides exhibiting headpin activity will be most preferred.

The term “functionally equivalent codon” is used herein to refer to codons that encode the same amino acid, such as the six codons for arginine and serine, and also refers to codons that encode biologically equivalent amino acids. For optimization of expression of headpin in human cells, the codons are shown in Table 1 in preference of use from left to right. Thus, the most preferred codon for alanine is thus “GCC”, and the least is “GCG” (see Table 1 below). Codon usage for various organisms and organelles can be found at the website http://www.kazusa.or.jp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria or chloroplasts, based on the preferred codon usage as would be known to those of ordinary skill in the art.

TABLE 1

Preferred Human DNA Codons

| | |
|---|---|
| Amino Acids | Codons |

| | | | | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferrably about 81% and about 90%; or even more preferably, between about 91% and about 99%; of nucleotides that are identical to the nucleotides of SEQ ID NO:1 will be sequences that are “essentially as set forth in SEQ ID NO:1”.

B. Nucleic Acid Hybidization

The nucleic acid sequences disclosed herein also have a variety of uses, such as for example, utility as probes or primers in nucleic acid hybridization embodiments.

Naturally, the present invention also encompasses DNA segments that are complementary, or essentially complementary, to the sequence set forth in SEQ ID NO:1. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under stringent conditions such as those described herein.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Accordingly, the nucleotide sequences of the disclosure may be used for their ability to selectively form duplex molecules with complementary stretches of genes or RNAs or to provide primers for amplification of DNA or RNA from tissues. Depending on the application envisioned, it is preferred to employ varying conditions of hybridization to achieve varying degrees of selectivity of probe towards target sequence.

The nucleic acid segments of the present invention, regardless of the length of the coding sequence itself, may be combined with other DNA sequences, such as promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

For example, nucleic acid fragments may be prepared that include a contiguous stretch of nucleotides identical to or complementary to SEQ ID NO:1, such as, for example, about 8, about 10 to about 14, or about 15 to about 20 nucleotides, and that are chromosome sized pieces, up to about 1,000,000, about 750,000, about 500,000, about 250,000, about 100,000, about 50,000, about 20,000, or about 10,000, or about 5,000 base pairs in length, with segments of about 3,000 being preferred in certain cases, as well as DNA segments with total lengths of about 1,000, about 500, about 200, about 100 and about 50 base pairs in length (including all intermediate lengths of these lengths listed above, i.e., any range derivable therein and any integer derivable therein such a range) are also contemplated to be useful.

For example, it will be readily understood that “intermediate lengths”, in these contexts, means any length between the quoted ranges, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105,.110, 115, 120, 130, 140, 150, 160, 170, 180, 190, including all integers through the 200-500; 500-1,000; 1,000-2,000; 2,000-3,000; 3,000-5,000; 5,000-10,000 ranges, up to and including sequences of about 12,001, 12,002, 13,001, 13,002, 15,000, 20,000 and the like.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1 to 15, 2 to 16, 3 to 17 . . . and/or so on. For a 20-mer, the nucleic segments correspond to bases 1 to 20, 2 to 21, 3 to 22 . . . and/or so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

The use of a hybridization probe of between 17 and 100 nucleotides in length, or in some aspect of the invention even up to 1-2 Kb or more in length, allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

In general, it is envisioned that the hybridization probes described herein will be useful both as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface to remove non-specifically bound probe molecules, hybridization is detected, or even quantified, by means of the label.

C. Nucleic Acid Amplification

Nucleic acid used as a template for amplification is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to headpin genes are contacted with the isolated nucleic acid under conditions that permit selective hybridization. The term “primer”, as defined herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology).

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, each incorporated herein by reference in entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641, filed Dec. 21, 1990, incorporated herein by reference. Polymerase chain reaction methodologies are well known in the art.

Another method for amplification is the ligase chain reaction (“LCR”), disclosed in EPA No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, incorporated herein by reference, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Gingeras et al., PCT Application WO 88/10315, incorporated herein by reference). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990, incorporated herein by reference).

Methods based on ligation of two (or more) oligonucleotides in the presence of nucleic acid having the sequence of the resulting “di-oligonucleotide”, thereby amplifying the di-oligonucleotide, may also be used in the amplification step of the present invention.

D. Nucleic Acid Detection

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means visible to the human eye or spectrophotometrically, to identify specific hybridization with complementary nucleic acid-containing samples.

In embodiments wherein nucleic acids are amplified, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods for genetic screening to accurately detect mutations in genomic DNA, cDNA or RNA samples may be employed, depending on the specific situation.

Historically, a number of different methods have been used to detect point mutations, including denaturing gradient gel electrophoresis (“DGGE”), restriction enzyme polymorphism analysis, chemical and enzymatic cleavage methods, and others. The more common procedures currently in use include direct sequencing of target regions amplified by PCR™ (see above) and single-strand conformation polymorphism analysis (“SSCP”).

Another method of screening for point mutations is based on RNase cleavage of base pair mismatches in RNA/DNA and RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single and multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. After the RNase cleavage reaction, the RNase is inactivated by proteolytic digestion and organic extraction, and the cleavage products are denatured by heating and analyzed by electrophoresis on denaturing polyacrylamide gels. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Currently available RNase mismatch cleavage assays, including those performed according to U.S. Pat. No. 4,946,773, require the use of radiolabeled RNA probes. Myers and Maniatis in U.S. Pat. No. 4,946,773 describe the detection of base pair mismatches using RNase A. Other investigators have described the use of an E. coli enzyme, RNase I, in mismatch assays. Because it has broader cleavage specificity than RNase A, RNase I would be a desirable enzyme to employ in the detection of base pair mismatches if components can be found to decrease the extent of non-specific cleavage and increase the frequency of cleavage of mismatches. The use of RNase I for mismatch detection is described in literature from Promega Biotech. Promega markets a kit containing RNase I that is shown in their literature to cleave three out of four known mismatches, provided the enzyme level is sufficiently high.

The RNase protection assay was first used to detect and map the ends of specific mRNA targets in solution. The assay relies on being able to easily generate high specific activity radiolabeled RNA probes complementary to the mRNA of interest by in vitro transcription. Originally, the templates for in vitro transcription were recombinant plasmids containing bacteriophage promoters. The probes are mixed with total cellular RNA samples to permit hybridization to their complementary targets, then the mixture is treated with RNase to degrade excess unhybridized probe. Also, as originally intended, the RNase used is specific for single-stranded RNA, so that hybridized double-stranded probe is protected from degradation. After inactivation and removal of the RNase, the protected probe (which is proportional in amount to the amount of target mRNA that was present) is recovered and analyzed on a polyacrylamide gel.

The RNase Protection assay was adapted for detection of single base mutations. In this type of RNase A mismatch cleavage assay, radiolabeled RNA probes transcribed in vitro from wild-type sequences, are hybridized to complementary target regions derived from test samples. The test target generally comprises DNA (either genomic DNA or DNA amplified by cloning in plasmids or by PCR™), although RNA targets (endogenous mRNA) have occasionally been used. If single nucleotide (or greater) sequence differences occur between the hybridized probe and target, the resulting disruption in Watson-Crick hydrogen bonding at that position (“mismatch”) can be recognized and cleaved in some cases by single-strand specific ribonuclease. To date, RNase A has been used almost exclusively for cleavage of single-base mismatches, although RNase I has recently been shown as useful also for mismatch cleavage. There are recent descriptions of using the MutS protein and other DNA-repair enzymes for detection of single-base mismatches.

E. Cloning Headpin Genes

The present invention contemplates cloning headpin genes or cDNAs from animal (e.g., mammalian) organisms. A technique often employed by those skilled in the art of protein production today is to obtain a so-called “recombinant” version of the protein, to express it in a recombinant cell and to obtain the protein, polypeptide or peptide from such cells. These techniques are based upon the “cloning” of a DNA molecule encoding the protein from a DNA library, i.e., on obtaining a specific DNA molecule distinct from other portions of DNA. This can be achieved by, for example, cloning a cDNA molecule, or cloning a genomic-like DNA molecule.

The first step in such cloning procedures is the screening of an appropriate DNA library, such as, for example, from a mouse, rat, monkey or human. The screening protocol may utilize nucleotide segments or probes that are designed to hybridize to cDNA or genomic sequences of headpins from protists. Additionally, antibodies designed to bind to the expressed headpin proteins, polypeptides, or peptides may be used as probes to screen an appropriate mammalian DNA expression library. Alternatively, activity assays may be employed. The operation of such screening protocols are well known to those of skill in the art and are described in detail in the scientific literature, for example, in Sambrook et al. (1989), incorporated herein by reference. Moreover, as the present invention encompasses the cloning of genomic segments as well as cDNA molecules, it is contemplated that suitable genomic cloning methods, as known to those in the art, may also be used.

As used herein “designed to hybridize” means a sequence selected for its likely ability to hybridize to a mammalian headpin gene, for example due to the expected high degree of homology between the hauman headpin gene and the headpin genes from other mammals. Also included are segments or probes altered to enhance their ability to hybridize to or bind to a mammalian headpin gene. Additionally, these regions of homology also include amino acid sequences of 4 or more consecutive amino acids selected and/or altered to increase conservation of the amino acid sequences in comparison to the same or similar region of residues in the same or related genes in one or more species. Such amino acid sequences may derived from amino acid sequences encoded by the headpin gene, and more particularly from the isolated sequences of SEQ ID NO:2.

General methods for screening a mammalian DNA library are exemplified by, but not limited to, the methods detailed in Example 1 herein below. Nucleotide probes may derived from nucleotide sequences from the human headpin sequence, and more particularly from the isolated sequences of SEQ ID NO:1. Such sequences may be used as probes for hybridization or oligonucleotide primers for PCR™. Designing such sequences may involve selection of regions of highly conserved nucleotide sequences between various species for a particular gene or related genes, relative to the general conservation of nucleotides of the gene or related genes in one or more species. Comparison of the amino acid sequences conserved between one or more species for a particular gene may also be used to determine a group of 4 or more consecutive amino acids that are conserved relative to the protein encoded by the gene or related genes. The nucleotide probe or primers may then be designed from the region of the gene that encodes the conserved sequence of amino acids.

One may also prepare fusion proteins, polypeptides and peptides, e.g., where the headpin proteinaceous material coding regions are aligned within the same expression unit with other proteins, polypeptides or peptides having desired functions, such as for purification or immunodetection purposes (e.g., proteinaceous compostions that may be purified by affinity chromatography and enzyme label coding regions, respectively).

Encompassed by the invention are DNA segments encoding relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO:2 and also larger polypeptides up to and including proteins corresponding to the full-length sequences set forth in SEQ ID NO:2, and any range derivable therein and any integer derivable therein such a range.

In addition to the “standard” DNA and RNA nucleotide bases, modified bases are also contemplated for use in particular applications of the present invention. A table of exemplary, but not limiting, modified bases is provided herein below.

TABLE 2
Modified Bases

| Abbr. | Modified base description |
|---|---|
| ac4c | 4-acetylcytidine |
| chm5u | 5-(carboxyhydroxylmethyl)uridine |
| Cm | 2′-O-methylcytidine |
| Cmnm5s2u | 5-carboxymethylaminomethyl- |
| | 2-thioridine |
| Cmnm5u | 5-carboxymethylaminomethyluridine |
| D | Dihydrouridine |
| Fm | 2′-O-methylpseudouridine |
| gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine- |
| | 6-yl)carbamoyl)threonine |
| Mt6a | N-((9-beta-D-ribofuranosylpurine-6- |
| | yl)N-methyl-carbamoyl)threonine |
| Mv | Uridine-5-oxyacetic acid |
| | Methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, |
| | (acp3)u |

II. Mutagenesis, Peptidomimetics and Rational Drug Design

It will also be understood that this invention is not limited to the particular nucleic acid and amino acid sequences of SEQ ID NO:1. Recombinant vectors and isolated DNA segments may therefore variously include these coding regions themselves, coding regions bearing selected alterations or modifications in the basic coding region, or they may encode larger polypeptides that nevertheless include such coding regions or may encode biologically functional equivalent proteins, polypeptides or peptides that have variant amino acids sequences.

The DNA segments of the present invention encompass biologically functional equivalent headpin proteins, polypeptides, and peptides. Such sequences may arise as a consequence of codon redundancy and functional equivalency that are known to occur naturally within nucleic acid sequences and the proteinaceous compositions thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements to the antigenicity of the proteinaceous composition or to test mutants in order to examine headpin activity at the molecular level.

Site-specific mutagenesis is a technique useful in the preparation of individual peptides, or biologically functional equivalent proteins, polypeptides or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired proteinaceous molecule. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

As modifications and changes may be made in the structure of the headpin genes, nucleic acids (e.g., nucleic acid segments) and proteinaceous molecules of the present invention, and still obtain molecules having like or otherwise desirable characteristics, such biologically functional equivalents are also encompassed within the present invention.

For example, certain amino acids may be substituted for other amino acids in a proteinaceous structure without appreciable loss of interactive binding capacity with structures such as, for example, antigen-binding regions of antibodies, binding sites on substrate molecules or receptors, or such like. Since it is the interactive capacity and nature of a proteinaceous molecule that defines that proteinaceous molecule's biological functional activity, certain amino acid sequence substitutions can be made in a proteinaceous molecule sequence (or, of course, its underlying DNA coding sequence) and nevertheless obtain a proteinaceous molecule with like (agonistic) properties. It is thus contemplated that various changes may be made in the sequence of headpin proteins, polypeptides or peptides, or the underlying nucleic acids, without appreciable loss of their biological utility or activity.

Equally, the same considerations may be employed to create a protein, polypeptide or peptide with countervailing, e.g., antagonistic properties. This is relevant to the present invention in which headpin mutants or analogues may be generated. For example, a headpin mutant may be generated and tested for headpin activity to identify those residues important for headpin activity. Headpin mutants may also be synthesized to reflect a headpin mutant that occurs in the human population and that is linked to the development of cancer. Such mutant proteinaceous molecules are particularly contemplated for use in generating mutant-specific antibodies and such mutant DNA segments may be used as mutant-specific probes and primers.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. A table of amino acids and their codons is presented herein above for use in such embodiments, as well as for other uses, such as in the design of probes and primers and the like.

In terms of functional equivalents, it is well understood by the skilled artisan that, inherent in the definition of a “biologically functional equivalent” protein, polypeptide, peptide, gene or nucleic acid, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted.

In particular, where shorter length peptides are concerned, it is contemplated that fewer amino acids changes should be made within the given peptide. Longer domains may have an intermediate number of changes. The full length protein will have the most tolerance for a larger number of changes. Of course, a plurality of distinct proteins/polypeptide/peptides with different substitutions may easily be made and used in accordance with the invention.

It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a proteinaceous molecule is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biological functional equivalent protein, polypeptide or peptide thereby created is intended for use in immunological embodiments, as in certain embodiments of the present invention. U.S. Patent 4,554,101, incorporated herein by reference, states that the greatest local average hydrophilicity of a proteinaceous molecule, as governed by the hydrophilicity of its adjacent amino acids, correlates with its immunogenicity and antigenicity, i.e., with a biological property of the proteinaceous molecule.

As detailed in U.S. Patent 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

In addition to the headpin peptidyl compounds described herein, it is contemplated that other sterically similar compounds may be formulated to mimic the key portions of the peptide structure. Such compounds, which may be termed peptidomimetics, may be used in the same manner as the peptides of the invention and hence are also functional equivalents.

Certain mimetics that mimic elements of proteinaceous molecule's secondary structure are described in Johnson et al. (1993). The underlying rationale behind the use of peptide mimetics is that the peptide backbone of proteinaceous molecules exists chiefly to orientate amino acid side chains in such a way as to facilitate molecular interactions, such as those of antibody and antigen. A peptide mimetic is thus designed to permit molecular interactions similar to the natural molecule.

Some successful applications of the peptide mimetic concept have focused on mimetics of β-turns within proteinaceous molecules, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

The generation of further structural equivalents or mimetics may be achieved by the techniques of modeling and chemical design known to those of skill in the art. The art of receptor modeling is now well known, and by such methods a chemical that binds headpin can be designed and then synthesized. It will be understood that all such sterically designed constructs fall within the scope of the present invention.

In addition to the 20 “standard” amino acids provided through the genetic code, modified or unusual amino acids are also contemplated for use in the present invention. A table of exemplary, but not limiting, modified or unusual amino acids is provided herein below.

TABLE 3
Modified and Unusual Amino Acids

| Abbr. | Amino Acid |
|---|---|
| Aad | 2-Aminoadipic acid |
| Baad | 3-Aminoadipic acid |
| Bala | Beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| Abbr. | Amino Acid |
| EtAsn | N-Ethylasparagine |
| Hyl | Hydroxylysine |
| aHyl | Allo-Hydroxylysine |
| 3Hyp | 3-Hydroxyproline |
| 4Hyp | 4-Hydroxyproline |
| Ide | Isodesmosine |
| aIle | Allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In one aspect, an compound may be designed by ratioal drug design to function as a headpin in inhibition serine proteases. The goal of rational drug design is to produce structural analogs of biologically active compounds. By creating such analogs, it is possible to fashion drugs which are more active or stable than the natural molecules, which have different susceptibility to alteration or which may affect the function of various other molecules. In one approach, one would generate a three-dimensional structure for the headpin protein of the invention or a fragment thereof. This could be accomplished by X-ray crystallography, computer modeling or by a combination of both approaches. An alternative approach, involves the random replacement of functional groups throughout the headpin protein, polypeptides or peptides, and the resulting affect on function determined.

It also is possible to isolate a headpin protein, polypeptide or peptide specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have enhanced and improved biological activity, for example, serine protease or tumor growth or metastasis inhibition, relative to a starting headpin proteinaceous sequences. By virtue of the ability to recombinatly produce sufficient amounts of the headpin proteins, polypeptides or peptides, crystallographic studies may be preformed to determine the most likely sites for mutagenesis and chemical mimicry. In addition, knowledge of the chemical characteristics of these compounds permits computer employed predictions of structure-function relationships. Computer models of various polypeptide and peptide structures are also available in the literature or computer databases. In a non-limiting example, the Entrez database (http://www.ncbi.nlm.nih.gov/Entrez/) may be used by one of ordinary skill in the art to identify target sequences and regions for mutagenesis.

III. Recombinant Vectors, Host Cells and Expression

Recombinant vectors form an important further aspect of the present invention. The term “expression vector or construct” means any type of genetic construct containing a nucleic acid coding for a gene product in which part or all of the nucleic acid encoding sequence is capable of being transcribed. The transcript may be translated into a proteinaceous molecule, but it need not be. Thus, in certain embodiments, expression includes both transcription of a gene and translation of a RNA into a gene product. In other embodiments, expression only includes transcription of the nucleic acid, for example, to generate antisense constructs.

Particularly useful vectors are contemplated to be those vectors in which the coding portion of the DNA segment, whether encoding a full length protein or smaller polypeptide or peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control” or “under transcriptional control” means that the promoter is in the correct location and orientation in relation to the nucleic acid to control RNA polymerase initiation and expression of the gene.

The promoter may be in the form of the promoter that is naturally associated with an headpin gene, as may be obtained by isolating the 5′ non-coding sequences located upstream of the coding segment or exon, for example, using recombinant cloning and/or PCR technology, in connection with the compositions disclosed herein (PCR™ technology is disclosed in U.S. Pat. No. 4,683,202 and U.S. Pat. No. 4,682,195, each incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with an headpin gene in its natural environment. Such promoters may include promoters normally associated with other genes, and/or promoters isolated from any other bacterial, viral, eukaryotic, protist, or mammalian cell, and/or promoters made by the hand of man that are not “naturally occurring”, i.e., containing difference elements from different promoters, or mutations that increase, decrease, or alter expression.

Naturally, it will be important to employ a promoter that effectively directs the expression of the DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of the introduced DNA segment, such as is advantageous in the large-scale production of recombinant proteins, polypeptides or peptides.

At least one module in a promoter generally functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase promoter, the spacing between promoter elements can be increased to 50 basepairs apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of the instant nucleic acids. The use of other viral or mammalian cellular or bacterial phage promoters which are well-known in the art to achieve expression are contemplated as well, provided that the levels of expression are sufficient for a given purpose. Tables 4 and 5 below list several elements/promoters which may be employed, in the context of the present invention, to regulate the expression of an headpin gene. This list is not intended to be exhaustive of all the possible elements involved in the promotion of expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 4
Promoter and Enhancer Elements

| Promoter/Enhancer | References |
|---|---|
| Immunoglobulin Heavy Chain | Banerji et al., 1983; Gilles et al., 1983; Grosschedl |
| | and Baltimore, 1985; Atchinson and Perry, 1986, 1987; |
| | Imler et al., 1987; Weinberger et al., 1984; |
| | Kiledjian et al., 1988; Porton et al.; 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al.; 1990 |
| HLA DQ a and DQ β | Sullivan and Peterlin, 1987 |
| β-Interferon | Goodbourn et al., 1986; Fujita et al., 1987; Goodbourn |
| | and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick and Benfield, 1989; |
| | Johnson et al., 1989 |
| Prealbumin (Transthyretin) | Costa et al., 1988 |
| Elastase I | Ornitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | |
| c-HA-ras | Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; |
| | Kuhl et al., 1987; Schaffner et al., 1988 |
| Polyoma | Swartzendruber and Lehman, 1975; Vasseur et al., 1980; |
| | Katinka et al., 1980, 1981; Tyndell et al., 1981; |
| | Dandolo et al., 1983; de Villiers et al., 1984; |
| | Hen et al., 1986; Satake et al., 1988; Campbell and |
| | Villarreal, 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; |
| | Choi et al., 1988; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; |
| | Hirochika et al., 1987; Stephens and Hentschel, 1987 |
| Hepatitis B Virus | Bulla and Siddiqui, 1986; Jameel and Siddiqui, 1986; |
| | Shaul and Ben-Levy, 1987; Spandau and Lee, 1988; |
| | Vannice and Levinson, 1988 |
| Human Immunodeficiency Virus | Muesing et al., 1987; Hauber and Cullan, 1988; |
| | Jakobovits et al., 1988; Feng and Holland, 1988; |
| | Takebe et al., 1988; Rosen et al., 1988; |
| | Berkhout et al., 1989; Laspia et al., 1989; Sharp and |
| | Marciniak, 1989; Braddock et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 5
Inducible Elements

| Element | Inducer | References |
|---|---|---|
| MT II | Phorbol Ester (TFA) | Palmiter et al., 1982; |
| | | Haslinger |
| | Heavy metals | and Karin, 1985; |
| | | Searle et al., 1985; |
| | | Stuart et al., 1985; |
| | | Imagawa et al., 1987, |
| | | Karin et al., 1987; |
| | | Angel et al., 1987b; |
| | | McNeall et al., 1989 |
| MMTV (mouse | Glucocorticoids | Huang et al., 1981; |
| mammary tumor | | Lee et al., 1981; |
| virus) | | Majors and |
| | | Varmus, 1983; |
| | | Chandler et al., 1983; |
| | | Lee et al., 1984; |
| | | Ponta et al., 1985; |
| | | Sakai et al., 1988 |
| β-Interferon | Poly(rI)x | Tavernier et al., 1983 |
| | Poly(rc) | |
| Adenovirus 5 E2 | Ela | Imperiale and |
| | | Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | |
| | Newcastle | |
| | Disease Virus | |
| GRP78 Gene | A23187 | Resendez et al., |
| | | 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | Ela, SV40 Large T | Taylor et al., 1989; |
| | Antigen | Taylor and |
| | | Kingston, 1990a, b |
| Proliferin | Phorbol Ester-TPA | Mordacq and Linzer, |
| | | 1989 |
| Tumor Necrosis Factor | FMA | Hensel et al., 1989 |
| Thyroid Stimulating | Thyroid Hormone | Chatterjee et al., |
| Hormone a Gene | | 1989 |

Turning to the expression of the headpin proteinaceous molecules of the present invention, once a suitable clone or clones have been obtained, whether they be cDNA based or genomic, one may proceed to prepare an expression system. The engineering of DNA segment(s) for expression in a prokaryotic or eukaryotic system may be performed by techniques generally known to those of skill in recombinant expression. It is believed that virtually any expression system may be employed in the expression of the proteinaceous molecules of the present invention.

Both cDNA and genomic sequences are suitable for eukaryotic expression, as the host cell will generally process the genomic transcripts to yield functional mRNA for translation into proteinaceous molecules. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude or more larger than the cDNA gene. However, it is contemplated that a genomic version of a particular gene may be employed where desired.

In expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

The term “antisense nucleic acid” is intended to refer to the oligonucleotides complementary to the base sequences of DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs may be designed to bind to the promoter and other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementary rules. That is, that the larger purines will base pair with the smaller pyrimidines to form only combinations of guanine paired with cytosine (G:C) and adenine paired with either thymine (A:T), in the case of DNA, or adenine paired with uracil (A:U) in the case of RNA.

As used herein, the terms “complementary” or “antisense sequences” mean nucleic acid sequences that are substantially complementary over their entire length and have very few base mismatches. For example, nucleic acid sequences of fifteen bases in length may be termed complementary when they have a complementary nucleotide at thirteen or fourteen positions with only single or double mismatches. Naturally, nucleic acid sequences which are “completely complementary” will be nucleic acid sequences which are entirely complementary throughout their entire length and have no base mismatches.

While all or part of the gene sequence may be employed in the context of antisense construction, statistically, any sequence 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting of the corresponding host cell gene simply by testing the constructs in vitro to determine whether the endogenous gene's function is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al, 1993).

As an alternative to targeted antisense delivery, targeted ribozymes may be used. The term “ribozyme” refers to an RNA-based enzyme capable of targeting and cleaving particular base sequences in oncogene DNA and RNA. Ribozymes either can be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

It is proposed that headpin proteins, polypeptides or peptides may be co-expressed with other selected proteinaceous molecules, wherein the proteinaceous molecules may be co-expressed in the same cell or headpin gene may be provided to a cell that already has another selected proteinaceous molecule. Co-expression may be achieved by co-transfecting the cell with two distinct recombinant vectors, each bearing a copy of either of the respective DNA. Alternatively, a single recombinant vector may be constructed to include the coding regions for both of the proteinaceous molecules, which could then be expressed in cells transfected with the single vector. In either event, the term “co-expression” herein refers to the expression of both the headpin gene and the other selected proteinaceous molecules in the same recombinant cell.

As used herein, the terms “engineered” and “recombinant” cells or host cells are intended to refer to a cell into which an exogenous DNA segment or gene, such as a cDNA or gene encoding a headpin protein, polypeptide or peptide has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant headpin protein, polypeptide or peptide, whether mutant or wild-type, in accordance with the present invention one would prepare an expression vector that comprises a wild-type, or mutant headpin proteinaceous molecule-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded recombinant protein, polypeptide or peptide. This is the meaning of “recombinant expression” in this context.

Many standard techniques are available to construct expression vectors containing the appropriate nucleic acids and transcriptional/translational control sequences in order to achieve protein, polypeptide or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

The following details concerning recombinant protein production in bacterial cells, such as E. coli, are provided by way of exemplary information on recombinant protein production in general, the adaptation of which to a particular recombinant expression system will be known to those of skill in the art.

Bacterial cells, for example, E. coli, containing the expression vector are grown in any of a number of suitable media, for example, LB. The expression of the recombinant proteinaceous molecule may be induced, e.g., by adding IPTG to the media or by switching incubation to a higher temperature. After culturing the bacteria for a further period, generally of between 2 and 24 hours, the cells are collected by centrifugation and washed to remove residual media.

The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed.

If the recombinant proteinaceous molecule is expressed in the inclusion bodies, as is the case in many instances, these can be washed in any of several solutions to remove some of the contaminating host proteins, then solubilized in solutions containing high concentrations of urea (e.g., 8M) or chaotropic agents such as guanidine hydrochloride in the presence of reducing agents, such as β-mercaptoethanol or DTT (dithiothreitol).

Under some circumstances, it may be advantageous to incubate the proteinaceous molecule for several hours under conditions suitable for the proteinaceous molecule to undergo a refolding process into a conformation which more closely resembles that of the native proteinaceous molecule. Such conditions generally include low proteinaceous molecule concentrations, less than 500 mg/ml, low levels of reducing agent, concentrations of urea less than 2 M and often the presence of reagents such as a mixture of reduced and oxidized glutathione which facilitate the interchange of disulfide bonds within the proteinaceous molecule.

The refolding process can be monitored, for example, by SDS-PAGE, or with antibodies specific for the native molecule (which can be obtained from animals vaccinated with the native molecule or smaller quantities of recombinant proteinaceous molecule). Following refolding, the proteinaceous molecule can then be purified further and separated from the refolding mixture by chromatography on any of several supports including ion exchange resins, gel permeation resins or on a variety of affinity columns.

For expression in Saccharomyces, the plasmid YRp7, for example, is commonly used. This plasmid already contains the trpl gene which provides a selection marker for a mutant strain of yeast lacking the ability to grow in tryptophan, for example ATCC No. 44076 or PEP4-1. The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more headpin protein, polypeptide or peptide coding sequences.

In a useful insect system, Autograph californica nuclear polyhedrosis virus (AcNPV) is used as a vector to express foreign genes. The virus grows in Spodoptera frugiperda cells. The headpin protein, polypeptide or peptide coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051, Smith, incorporated herein by reference).

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of proteinaceous products may be important for the function of the proteinaceous molecule.

Different host cells have characteristic and specific mechanisms for the post-translational processing and modification of proteinaceous molecules. Appropriate cells lines or host systems can be chosen to ensure the correct modification and processing of the foreign proteinaceous molecule expressed.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the headpin gene, provided such control sequences are compatible with the host cell systems.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1, E3, or E4) will result in a recombinant virus that is viable and capable of expressing headpin proteins, polypeptides or peptides in infected hosts.

Specific initiation signals may also be required for efficient translation of headpin protein, polypeptide or peptide coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements and transcription terminators.

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the proteinaceous molecule at a position prior to transcription termination.

For long-term, high-yield production of a recombinant headpin protein, polypeptide or peptide, stable expression is preferred. For example, cell lines that stably express constructs encoding an headpin protein, polypeptide or peptide may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including, but not limited to, the herpes simplex virus thymidine kinase (tk), hypoxanthine-guanine phosphoribosyltransferase (hgprt) and adenine phosphoribosyltransferase (aprt) genes, in tk⁻, hgprt⁻ or aprt⁻ cells, respectively. Also, antimetabolite resistance can be used as the basis of selection for dihydrofolate reductase (dhfr), that confers resistance to methotrexate; gpt, that confers resistance to mycophenolic acid; neomycin (neo), that confers resistance to the aminoglycoside G-418; and hygromycin (hygro), that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower proteinaceous molecule production than adherent cells.

Large scale suspension culture of mammalian cells in stirred tanks is a common method for production of recombinant proteinaceous molecules. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

It is contemplated that the headpin proteins, polypeptides or peptides of the invention may be “overexpressed”, i.e., expressed in increased levels relative to its natural expression in cells. Such overexpression may be assessed by a variety of methods, including radio-labeling and/or proteinaceous molecule purification. However, simple and direct methods are preferred, for example, those involving SDS/PAGE and proteinaceous composition staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific proteinaceous molecule in relation to the other proteins produced by the host cell and, e.g., visible on a gel.

IV. Methods of Gene Transfer

In order to mediate the effect of transgene expression in a cell, it will be necessary to transfer the expression constructs (e.g., a therapeutic construct) of the present invention into a cell. Such transfer may employ viral or non-viral methods of gene transfer. This section provides a discussion of methods and compositions of gene or nucleic acid transfer.

### 1. Viral Vector-Mediated Transfer

The mammalian headpin genes are incorporated into an adenoviral infectious particle to mediate gene transfer to a cell. Additional expression constructs encoding other therapeutic agents as described herein may also be transferred via viral transduction using infectious viral particles, for example, by transformation with an adenovirus vector of the present invention as described herein below. Alternatively, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Though adenovirus is exemplified, the present methods may be advantageously employed with other viral vectors, as discussed below.

Adenovirus. Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

Retrovirus. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

Adeno-associated Virus. AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chattejee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

Other Viral Vectors. Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

### 2. Non-viral Transfer

DNA constructs of the present invention are generally delivered to a cell, in certain situations, the nucleic acid to be transferred is non-infectious, and can be transferred using non-viral methods.

Several non-viral methods for the transfer of expression constructs into cultured mammalian cells are contemplated by the present invention. Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimeret al., 1987); by liposome mediated transfection (Nicolau and Sene, 1982; Fraleyet al., 1979; Nicolauet al., 1987; Wonget al., 1980; Kanedaetal., 1989; Katoetal., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Once the construct has been delivered into the cell the nucleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM may also be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

V. Headpin Proteins, Polypeptides, and Peptides

The present invention also provides purified, and in preferred embodiments, substantially purified mammalian headpin proteins, polypeptides, or peptides. The term “purified mammalian headpin proteins, polypeptides, or peptides” as used herein, is intended to refer to an headpin proteinaceous composition, isolatable from mammalian cells or recombinant host cells, wherein the headpin protein, polypeptide, or peptide is purified to any degree relative to its naturally-obtainable state, i.e., relative to its purity within a cellular extract. A purified headpin protein, polypeptide, or peptide therefore also refers to a wild-type or mutant headpin protein, polypeptide, or peptide free from the environment in which it naturally occurs.

The headpin proteins may be full length proteins, such as being 391 amino acids in length. The headpin proteins, polypeptides and peptides may also be less then full length proteins, such as individual polypeptide, domains, regions or even epitopic peptides. Where less than full length headpin proteins are concerned the most preferred will be those containing predicted immunogenic sites and those containing the functional domains identified herein.

Encompassed by the invention are proteinaceous segments of relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO:2, and also larger polypeptides of from about 51, about 52, about 53, about 54, about 55, about 56, about 57, about 58, about 59, about 60, about 65, about 70, about 75, about 80, about 85, about 90, about 95, about 100, about 110, about 120, about 130, about 140, about 150, about 160, about 170, about 180, about 190, about 200, about 220, about 240, about 260, about 280, about 300, about 320, about 340, about 360, about 380, up to and including proteins corresponding to the full-length sequences set forth in SEQ ID NO:2.

Generally, “purified” will refer to an headpin protein, polypeptide, or peptide composition that has been subjected to fractionation to remove various non-headpin protein, polypeptide, or peptide, and which composition substantially retains its headpin activity, as may be assessed, for example, by the headpin assay, as described herein below.

Where the term “substantially purified” is used, this will refer to a composition in which the headpin protein, polypeptide, or peptide forms the major component of the composition, such as constituting about 50% of the proteinaceous molecules in the composition or more. In preferred embodiments, a substantially purified proteinaceous molecule will constitute more than 60%, 70%, 80%, 90%, 95%, 99% or even more of the proteinaceous molecules in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of headpin proteins, polypeptides, or peptides will be known to those of skill in the art in light of the present disclosure. These include, for example, determining the specific headpin proteinaceous molecule's activity of a fraction, or assessing the number of proteins, polypeptides and peptides within a fraction by gel electrophoresis. Assessing the number of proteinaceous molecules within a fraction by SDS/PAGE analysis will often be preferred in the context of the present invention as this is straightforward.

To purify an headpin protein, polypeptide, or peptide a natural or recombinant composition comprising at least some headpin proteins, polypeptides, or peptides will be subjected to fractionation to remove various non-headpin components from the composition. In addition to those techniques described in detail herein below, various other techniques suitable for use in proteinaceous molecule purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of an headpin fusion protein using a specific binding partner. Such purification methods are routine in the art. As the present invention provides DNA sequences for headpin proteins, any fusion protein purification method can now be practiced. This is exemplified by the generation of an headpin-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography.

The exemplary purification methods disclosed herein represent exemplary methods to prepare a substantially purified headpin protein, polypeptide, or polypeptide. These methods are preferred as they result in the substantial purification of the headpin protein, polypeptide or peptide in yields sufficient for further characterization and use. However, given the DNA and proteinaceous molecules provided by the present invention, any purification method can now be employed.

Although preferred for use in certain embodiments, there is no general requirement that the headpin protein, polypeptide, or peptide always be provided in their most purified state. Indeed, it is contemplated that less substantially purified headpin protein, polypeptide or peptide, which are nonetheless enriched in headpin proteinaceous compositions, relative to the natural state, will have utility in certain embodiments. These include, for example, antibody generation where subsequent screening assays using purified headpin proteinaceous molecules are conducted.

Methods exhibiting a lower degree of relative purification may have advantages in total recovery of proteinaceous molecule product, or in maintaining the activity of an expressed proteinaceous molecule. Inactive products also have utility in certain embodiments, such as, e.g., in antibody generation.

VI. Antibodies to Headpin Proteins

A. Epitopic Core Sequences

Peptides corresponding to one or more antigenic determinants, or “epitopic core regions”, of the headpin proteins of the present invention can also be prepared. Such peptides should generally be at least five or six amino acid residues in length, will preferably be about 10, 15, 20, 25 or about 30 amino acid residues in length, and may contain up to about 35 to about 50 residues or so.

Synthetic peptides will generally be about 35 residues long, which is the approximate upper length limit of automated peptide synthesis machines, such as those available from Applied Biosystems (Foster City, Calif.). Longer peptides may also be prepared, e.g., by recombinant means.

U.S. Pat. No. 4,554,101, (Hopp) incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the headpin sequence disclosed herein in SEQ ID NO:2.

Numerous scientific publications have also been devoted to the prediction of secondary structure, and to the identification of epitopes, from analyses of amino acid sequences (Chou & Fasman, 1974a,b; 1978a,b, 1979). Any of these may be used, if desired, to supplement the teachings of Hopp in U.S. Pat. No. 4,554,101.

Moreover, computer programs are currently available to assist with predicting antigenic portions and epitopic core regions of proteinaceous molecules. Examples include those programs based upon the Jameson-Wolf analysis (Jameson & Wolf, 1988; Wolf et al., 1988), the program PepPlot® (Brutlag et al., 1990; Weinberger et al., 1985), and other new programs for proteinaceous molecule tertiary structure prediction (Fetrow and Bryant, 1993). Another commercially available software program capable of carrying out such analyses is MacVector (IBI, New Haven, Conn.).

In further embodiments, major antigenic determinants of a polypeptide may be identified by an empirical approach in which portions of the gene encoding the polypeptide are expressed in a recombinant host, and the resulting proteinaceous molecules tested for their ability to elicit an immune response. For example, PCR™ can be used to prepare a range of peptides lacking successively longer fragments of the C-terminus of the proteinaceous molecule. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs™ system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants can also be constructed and inserted into expression vectors by standard methods, for example, using PCR™ cloning methodology.

The use of such small peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

B. Antibody Generation

In certain embodiments, the present invention provides antibodies that bind with high specificity to the headpin proteinaceous molecules provided herein. Thus, antibodies that bind to the proteinaceous products of the isolated nucleic acid sequences of SEQ ID NO:1 are provided. As detailed above, in addition to antibodies generated against the full length proteins, antibodies may also be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

The methods for generating monoclonal antibodies (MAbs) generally begin along the same lines as those for preparing polyclonal antibodies. Briefly, a polyclonal antibody is prepared by immunizing an animal with an immunogenic headpin proteinaceous composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, Pennasylvania); low-dose Cyclophosphamide (CYP; 300 mg/m²) (Johnson/ Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate - and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified headpin protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8.63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a- population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as PLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

C. Antibody Conjugates

The present invention further provides antibodies against headpin proteinaceous molecules, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Antibody conjugates are thus preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging”. Again, antibody-directed imaging is less preferred for use with this invention.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium^{111,} ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled monoclonal antibodies of the present invention may be produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-^{99m} by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA). Also contemplated for use are fluorescent labels, including rhodamine, fluorescein isothiocyanate and renographin.

The much preferred antibody conjugates of the present invention are those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

D. Immunodetection Methods

In still further embodiments, the present invention concerns immunodetection methods for binding, purifying, removing, quantifying or otherwise generally detecting biological components such as headpin proteinaceous components. The headpin antibodies prepared in accordance with the present invention may be employed to detect wild-type or mutant headpin proteins, polypeptides or peptides. As described throughout the present application, the use of wild-type or mutant headpin specific antibodies is contemplated. The steps of various useful immunodetection methods have been described in the scientific literature, such as, e.g., Nakamura et al. (1987), incorporated herein by reference.

In general, the immunobinding methods include obtaining a sample suspected of containing an headpin protein, polypeptide or peptide, and contacting the sample with a first anti-headpin antibody in accordance with the present invention, as the case may be, under conditions effective to allow the formation of immunocomplexes.

These methods include methods for purifying wild-type or mutant headpin proteins, polypeptides or peptides as may be employed in purifying wild-type or mutant headpin proteins, polypeptides or peptides from patients' samples or for purifying recombinantly expressed wild-type or mutant headpin proteins, polypeptides or peptides. In these instances, the antibody removes the antigenic wild-type or mutant headpin protein, polypeptide or peptide component from a sample. The antibody will preferably be linked to a solid support, such as in the form of a column matrix, and the sample suspected of containing the wild-type or mutant headpin protein antigenic component will be applied to the immobilized antibody. The unwanted components will be washed from the column, leaving the antigen immunocomplexed to the immobilized antibody, which wild-type or mutant headpin protein, polypeptide or peptide antigen is then collected by removing the wild-type or mutant headpin protein, polypeptide or peptide from the column.

The immunobinding methods also include methods for detecting or quantifying the amount of a wild-type or mutant headpin proteinaceous reactive component in a sample, which methods require the detection or quantification of any immune complexes formed during the binding process. Here, one would obtain a sample suspected of containing a wild-type or mutant headpin protein, polypeptide or peptide, and contact the sample with an antibody against wild-type or mutant headpin, and then detect or quantify the amount of immune complexes formed under the specific conditions.

In terms of antigen detection, the biological sample analyzed may be any sample that is suspected of containing a wild-type or mutant headpin proteinaceous molecule-specific antigen, such as a diseased urogenital tract tissue section, secretion or specimen, separated or purified forms of any of the above wild-type or mutant headpin proteinaceous-containing compositions.

Contacting the chosen biological sample with the antibody under conditions effective and for a period of time sufficient to allow the formation of immune complexes (primary immune complexes) is generally a matter of simply adding the antibody composition to the sample and incubating the mixture for a period of time lone enough for the antibodies to form immune complexes with, i.e., to bind to, any headpin antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological or enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The headpin antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

### 1. ELISAs

As detailed above, immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the anti-headpin antibodies of the invention are immobilized onto a selected surface exhibiting protein affinity, such as a well in a polystyrene microtiter plate. Then, a test composition suspected of containing the wild-type or mutant headpin antigen, such as a clinical sample, is added to the wells. After binding and washing to remove non-specifically bound immune complexes, the bound wild-type or mutant headpin protein, polypeptide or peptide antigen may be detected. Detection is generally achieved by the addition of another anti-headpin antibody that is linked to a detectable label. This type of ELISA is a simple “sandwich ELISA”. Detection may also be achieved by the addition of a second anti-headpin antibody, followed by the addition of a third antibody that has binding affinity for the second antibody, with the third antibody being linked to a detectable label.

In another exemplary ELISA, the samples suspected of containing the wild-type or mutant headpin antigen are immobilized onto the well surface and then contacted with the anti-headpin antibodies of the invention. After binding and washing to remove non-specifically bound immune complexes, the bound anti-headpin antibodies are detected. Where the initial anti-headpin antibodies are linked to a detectable label, the immune complexes may be detected directly. Again, the immune complexes may be detected using a second antibody that has binding affinity for the first anti-headpin antibody, with the second antibody being linked to a detectable label.

Another ELISA in which the wild-type or mutant headpin proteins, polypeptides or peptides are immobilized, involves the use of antibody competition in the detection. In this ELISA, labeled antibodies against wild-type or mutant headpin protein, polypeptide or peptides are added to the wells, allowed to bind, and detected by means of their label. The amount of wild-type or mutant headpin antigen in an unknown sample is then determined by mixing the sample with the labeled antibodies against wild-type or mutant headpin before or during incubation with coated wells. The presence of wild-type or mutant headpin proteinaceous molecule in the sample acts to reduce the amount of antibody against wild-type or mutant headpin proteinaceous molecule available for binding to the well and thus reduces the ultimate signal. This is also appropriate for detecting antibodies against wild-type or mutant headpin protein, polypeptide or peptide in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a proteinaceous molecule or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid (ABTS) and H₂O₂, in the case of peroxidase as the enzyme label. Quantification is then achieved by measuring the degree of color generation, e.g., using a visible spectra spectrophotometer.

### 2. Immunohistochemistry

The antibodies of the present invention may also be used in conjunction with both fresh-frozen and formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

VII. Diagnostics and Screens for Mammalian Headpin

A. Diagnostics

As with the therapeutic methods of the present invention, the diagnostic methods are based upon the novel gene encoding headpin, which encode a protein that is predicted to have serine protease inhibitor activity. The diagnostic methods of the present invention generally involve determining either the type or the amount of a wild-type or mutant headpin proteinaceous molecule present within a biological sample from a patient suspected of having cancer. Irrespective of the actual role of headpin in the etiology of cancer, it will be understood that the detection of a mutant form of headpin is likely to be diagnostic of cancer, and that the detection of altered amounts of headpin, either at the mRNA or protein level, is also likely to have diagnostic implications, particularly where there is a reasonably significant difference in amounts.

The finding of a decreased amount of headpin in one, or preferably more, cancerous samples, in comparison to the amount within a sample from a control sample, will be indicative of the role of headpin in a particular disease. Following which, disease in others would be similarly diagnosed by detecting a decreased amount of headpin in a sample. The finding of a increased amount of headpin in one, or preferably more, patients, in comparison to the amount within a sample from a control subject, will be indicative of the role of the headpin in a particular disease. Following which, disease in others would be similarly diagnosed by detecting a increased amount of headpin in a sample.

The type or amount of headpin proteinaceous molecule present within a biological sample, such as a tissue sample, secretion, or body fluid, may be determined by means of a molecular biological assay to determine the level of a nucleic acid that encodes such an headpin proteinaceous molecule, or by means of an immunoassay to determine the level of the protein, polypeptide or peptide itself. Any of the foregoing nucleic acid detection methods or immunodetection methods may be employed as a diagnostic methods in the context of the present invention.

B. Modulators and Screening Assays

In still further embodiments, the present invention provides methods for identifying new compounds that modulate headpin activity, which may be termed as “candidate substances.” “Modulating compounds” or “compounds that modulate headpin activity” is meant to refer to substances that enhance, inhibit, or alter the activity of headpin. Such altered activity includes, but is not limited to, changes in binding preferences for target substrates, particularly for serine proteases, and changes in proteinaceous molecule-proteinaceous molecule interactions of headpin that may occur. It is contemplated that such screening techniques will prove useful in the general identification of any compound that will serve the purpose of modulating headpin activity.

Headpin modulators identified will have utility in methods involved in serine proteases, and are also contemplated for therapeutic uses. Modulators that affect headpin affinity for the serine proteases or headpin proteinaceous molecule-proteinaceous molecule interaction with other proteins or proteases, are also contemplated. For example, the ability to specifically modulate headpin activity is envisioned to be useful in cancer. Further, the impact of any possible adverse effects by a modulator of headpin activity can be limited or otherwise controlled by the more specific administration of the modulator to a tumor site, such as by direct application to a tumor or cancerous tissues.

It is further contemplated that useful compounds in this regard will in no way be limited to proteinaceous or peptidyl compounds. In fact, it may prove to be the case that the most useful pharmacological compounds for identification through application of the screening assays will be non-peptidyl in nature and, e.g., which will serve to modulate headpin activity through a tight binding or other chemical interaction. Candidate substances may be obtained from libraries of synthetic chemicals, or from natural samples, such as rain forest and marine samples.

### 1. Modulation of Headpin

To identify a headpin modulator using a headpin protease inhibitor assay, one would simply conduct parallel or otherwise comparatively controlled protease inhibitor assays and identify a compound that modulates headpin protease inhibitor acivity. The candidate screening assay is quite simple to set up and perform. After obtaining a relatively purified preparation of headpin protein, polypeptide or peptide, either from native or recombinant sources, one will simply admix a candidate substance with the headpin preparation, under conditions that would allow headpin to perform its function but for inclusion of a modulating substance.

For example, one will typically desire to include within the admixture an amount of a serine protease, although other substrates may be used, such as other proteases. In any event, one would measure the ability of the candidate substance to alter protease inhibition by the headpin protein, polypeptide, or peptide in the presence of the candidate substance. In general, one will desire to measure or otherwise determine the activity of the relatively purified headpin in the absence of the added candidate substance relative to the activity in the presence of the candidate substance in order to assess the relative modulating capability of the candidate substance.

Serine protease assays are well known in the prior art. U.S. Pat. No. 5,925,553 and U.S. Pat. No. 5,057,414 both describe assay methods for serine proteases and serine protease inhibitors, and are incorporated herein by reference. It is understood that many versions of this assay and the incorporation of serine protease inhibitors and modulators thereof are also possible and known to those of skill in the art. Such assays would be the basis for examining candidate modulators of headpin and its interaction with a serine protease as described in this application. Modulators of serine proteases and serine protease inhibitors and methods for their identification are also known to those of skill in the art. U.S. Pat. No. 5,602,253 and U.S. Pat. No. 5,866,413, incorporated herein by reference, describe novel modulators of serine protease inhibitors.

### 2. Second Generation Modulators

In addition to the modulating compounds initially identified, it is contemplated that other sterically similar compounds may be formulated to mimic the key portions of the structure of the modulators. Such compounds, which may include peptidomimetics of peptide modulators, may be used in the same manner as the initial modulators.

Certain mimetics that mimic elements of a proteinaceous molecule's secondary structure are designed using the rationale that the peptide backbone of proteinaceous molecules exists chiefly to orientate amino acid side chains in such a way as to facilitate molecular interactions. A peptide mimetic is thus designed to permit molecular interactions similar to the natural molecule.

Some successful applications of the peptide mimetic concept have focused on mimetics of β-turns within proteinaceous molecules, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

The generation of further structural equivalents or mimetics may be achieved by the techniques of modeling and chemical design known to those of skill in the art. The art of computer-based chemical modeling is now well known. Using such methods, a chemical that specifically modulates mammalian headpin activity can be designed, and then synthesized, following the initial identification of a compound that modulates mammalian headpin activity, but that is not specific or sufficiently specific to other human or animal headpins or segments thereof. It will be understood that all such sterically similar constructs and second generation molecules fall within the scope of the present invention.

VIII. Methods of Treating Cancer

In a particular aspect, the present invention provides methods for the treatment of various malignancies. Treatment methods will involve treating an individual with an effective amount of a viral particle, as described above, containing a gene encoding a mammalian headpin. Alternatively, treatment methods will involve treating an individual with an effective amount of a mammalian headpin proteinaceous composition. Also contemplated are treatment methods that involve treating a n individual with an effective amount of a mammalian headpin modulator composition. An effective amount is described, generally, as that amount sufficient to detectably and repeatedly to induce apoptosis, inhibit cell division, inhibit metastatic potential, reduce tumor burden, increase sensitivity to chemotherapy or radiotherapy, kill a cancer cell, inhibit the growth of a cancer cell, or induce tumor regression.

To kill cells, inhibit cell growth, inhibit metastasis, decrease tumor size and otherwise reverse or reduce the malignant phenotype of tumor cells, using the methods and compositions of the present invention, one would generally contact a “target” cell with the therapeutic viral, proteinaceous molecule, or modulator composition. This may be combined with compositions comprising other agents effective in the treatment of cancer. These compositions would be provided in a combined amount effective to kill or inhibit proliferation of the cell. This process may involve contacting the cells with the viral, proteinaceous molecule, or modulator composition and the agent(s) or factor(s) at the same time. This may be achieved by contacting the cell with a single composition or pharmacological formulation that includes both agents, or by contacting the cell with two distinct compositions or formulations, at the same time, wherein one composition includes the viral, proteinaceous molecule, or modulator composition and the other includes the second agent.

Alternatively, the treatment may precede or follow the other agent treatment by intervals ranging from minutes to weeks. In embodiments where the other agent and viral or proteinaceous composition are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 h of each other and, more preferably, within about 6-12 h of each other, with a delay time of only about 12 h being most preferred. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Administration of the therapeutic viral or proteinaceous composition of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described treatments.

Where clinical application of a composition is contemplated, it will be necessary to prepare the complex as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Aqueous compositions of the present invention comprise an effective amount of the compound, dissolved or dispersed in a pharmaceutically acceptable carrier or aqueous medium. Such compositions can also be referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The compositions of the present invention may include classic pharmaceutical preparations. Dispersions also can be prepared in glycerol, liquid polyethylene glycols, and mixtures thereof and in oils. Under ordinary conditions of storage and use, these preparations contain a preservative to prevent the growth of microorganisms.

Depending on the particular cancer to be, administration of therapeutic compositions according to the present invention will be via any common route so long as the target tissue is available via that route. This includes oral, nasal, buccal, rectal, vaginal or topical. Topical administration would be particularly advantageous for treatment of skin cancers. Alternatively, administration will be by orthotopic, intradermal, subcutaneous, intramuscular, intraperitoneal or intravenous injection. Such compositions would normally be administered as pharmaceutically acceptable compositions that include physiologically acceptable carriers, buffers or other excipients.

In certain embodiments, ex vivo therapies also are contemplated. Ex vivo therapies involve the removal, from a patient, of target cells. The cells are treated outside the patient's body and then returned. One example of ex vivo therapy would involve a variation of autologous bone marrow transplant. Many times, ABMT fails because some cancer cells are present in the withdrawn bone marrow, and return of the bone marrow to the treated patient results in repopulation of the patient with cancer cells. In one embodiment, however, the withdrawn bone marrow cells could be treated while outside the patient with an viral particle that targets and kills the cancer cell. Once the bone marrow cells are “purged,” they can be reintroduced into the patient.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

Preferably, patients will have adequate bone marrow function (defined as a peripheral absolute granulocyte count of >2,000/mm3 and a platelet count of 100,000/ mm³), adequate liver function (bilirubin<1.5 mg/dl) and adequate renal function (creatinine<1.5 mg/dl).

One of the preferred embodiments of the present invention involves the use of viral vectors to deliver therapeutic genes to cancer cells. Alternatively, another embodiment of the present invention involves the use of therapeutic proteinaceous compositions. Target cancer cells include cancers of the lung, brain, prostate, kidney, liver, ovary, breast, skin, stomach, esophagus, head and neck, testicles, colon, cervix, lymphatic system and blood. Of particular interest are skin cancers and squamous carcinomas.

According to the present invention, one may treat the cancer by directly injection a tumor with the viral vector or proteinaceous composition. Alternatively, the tumor may be infused or perfused with the vector or proteinaceous compostion using any suitable delivery vehicle. Local or regional administration, with respect to the tumor, also is contemplated. Finally, systemic administration may be performed. Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles or proteinaceous composition may advantageously be contacted by administering multiple injections to the tumor, spaced at approximately 1 cm intervals.

In certain embodiments, the tumor being treated may not, at least initially, be resectable. Treatments with therapeutic viral constructs or proteinaceous compositions may increase the resectability of the tumor due to shrinkage at the margins or by elimination of certain particularly invasive portions. Following treatments, resection may be possible. Additional viral or proteinaceous compositon's treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

A. Combination Therapies

In order to increase the effectiveness of a headpin nucleic acid construct or proteinaceous molecule, it may be desirable to combine these compositions of the with an agent effective in the treatment of hyperproliferative disease, such as, for example, an anti-cancer agent. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with an headpin nucleic acid construct or proteinaceous molecule effective to kill or inhibit proliferation of a cancer cell. This process may involve contacting the cell(s) with an agent(s) and the headpin nucleic acid construct or proteinaceous molecule at the same time. This may be achieved by contacting the cell, tissue or organism with a single composition or pharmacological formulation that includes both a headpin nucleic acid construct or proteinaceous molecule and one or more agents, or by contacting the cell with two or more distinct compositions or formulations, at the same time, wherein one composition includes a headpin nucleic acid construct or proteinaceous molecule and the other includes one or more agents.

The terms “contacted” and “exposed,” when applied to a cell, tissue or organism, are used herein to describe the process by which a therapeutic construct or proteinaceos composition of the invention and/or another agent, such as for example a chemotherapeutic or radiotherapeutic agent, are delivered to a target cell, tissue or organism or are placed in direct juxtaposition with the target cell, tissue or organism. To achieve cell killing or stasis, the headpin nucleic acid construct or proteinaceous molecule and/or additional agent(s) are delivered to one or more cells in a combined amount effective to kill the cell(s) or prevent them from dividing.

The headpin nucleic acid construct or proteinaceous molecule may precede, be co-current with and/or follow the other agent(s) by intervals ranging from minutes to weeks. In embodiments where the headpin nucleic acid construct or proteinaceous molecule, and other agent(s) are applied separately to a cell, tissue or organism, one would generally ensure that a significant period of time did not expire between the time of each delivery, such that the headpin nucleic acid construct or proteinaceous molecule and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the headpin nucleic acid construct or proteinaceous molecule. In other aspects, one or more agents may be administered within of from about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, to about 48 hours or more prior to and/or after administering the headpin nucleic acid construct or proteinaceous molecule. In certain other embodiments, an agent may be administered within of from about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20, to about 21 days prior to and/or after administering the headpin nucleic acid construct or proteinaceous molecule. In some situations, it may be desirable to extend the time period for treatment significantly, however, where several weeks (e.g., about 1, about 2, about 3, about 4, about 5, about 6, about 7 or about 8 weeks or more) lapse between the respective administrations.

Various combinations may be employed, headpin gene, proteinaceous molecule, or modulator therapy is “A” and the radio- or chemotherapeutic agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the composition of the preseent invention to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention. For example, a modified ONYX-15 adenovirus has recently been shown to have effectiveness in treating head and neck tumors in combination with cisplatin and 5-fluorouracil (Khuri et al., 2000). It is contemplated that the compositions of the present invention may be combined with such agents, or other agents described herein, in the treatment of diseases, including cancer.

### 1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabien, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat, for example, chronic leukemia, non-Hodgkin's lymphoma, Hodgkin's disease, multiple myeloma, and particular cancers of the breast, lung, and ovary. An alkylating agent, may include, but is not limited to, a nitrogen mustard, an ethylenimene, a methylmelamine, an alkyl sulfonate, a nitrosourea or a triazines.

They include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

i. Nitrogen Mustards

A nitrogen mustard may be, but is not limited to, mechlorethamine (HN₂), which is used for Hodgkin's disease and non-Hodgkin's lymphomas; cyclophosphamide and/or ifosfamide, which are used in treating such cancers as acute or chronic lymphocytic leukemias, Hodgkin's disease, non-Hodgkin's lymphomas, multiple myeloma, neuroblastoma, breast, ovary, lung, Wilm's tumor, cervix testis and soft tissue sarcomas; melphalan (L-sarcolysin), which has been used to treat such cancers as multiple myeloma, breast and ovary; and chlorambucil, which has been used to treat diseases such as, for example, chronic lymphatic (lymphocytic) leukemia, malignant lymphomas including lymphosarcoma, giant follicular lymphoma , Hodgkin's disease and non-Hodgkin's lymphomas.

a. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. For example, after a single oral doses of about 0.6 mg/kg to about 1.2 mg/kg, peak plasma chlorambucil levels are reached within one hour and the terminal half-life of the parent drug is estimated at about 1.5 hours. About 0.1 mg/kg/day to about 0.2 mg/kg/day or about 3 6 mg/m²/day to about 6 mg/m²/day or alternatively about 0.4 mg/kg may be used for antineoplastic treatment. Chlorambucil is not curative by itself but may produce clinically useful palliation.

b. Cyclophosphamide

Cyclophosphamide is 2H- 1,3,2-Oxazaphosphorin-2-amine, N,N-bis(2-chloroethyl)tetrahydro-,2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable oral doses for adults include, for example, about 1 mg/kg/day to about 5 mg/kg/day (usually in combination), depending upon gastrointestinal tolerance; or about 1 mg/kg/day to about 2 mg/kg/day; intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. In some aspects, a dose of about 250 mg/kg/day may be administered as an antineoplastic. Because of gastrointestinal adverse effects, the intravenous route is preferred for loading. During maintenance, a leukocyte count of about 3000/mm3 to 4000/mm³ usually is desired. The drug also sometimes is administered intramuscularly, by infiltration or into body cavities. It is available in dosage forms for injection of about 100 mg, about 200 mg and about 500 mg, and tablets of about 25 mg and about 50 mg.

C. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of about 2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of about 0.2 mg/kg daily for five days as a single course. Courses are repeated about every four to five weeks depending upon hematologic tolerance (Young et al., 1978). Alternatively in certain embodiments, the dose of melphalan used could be as low as about 0.05 mg/kg/day or as high as about 3 mg/kg/day or greater.

ii. Ethylenimenes and Methymelamines

An ethylenimene and/or a methylmelamine include, but are not limited to, hexamethylmelamine, used to treat ovary cancer; and thiotepa, which has been used to treat bladder, breast and ovary cancer.

iii. Alkyl Sulfonates

An alkyl sulfonate includes but is not limited to such drugs as busulfan, which has been used to treat chronic granulocytic leukemia.

Busulfan (also known as myleran) is a bifunctional alkylating agent. Busulfan is known chemically as 1,4-butanediol dimethanesulfonate. Busulfan is available in tablet form for oral administration, wherein for example, each scored tablet contains about 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. Busulfan has been shown to be superior to splenic irradiation with respect to survival times and maintenance of hemoglobin levels, and to be equivalent to irradiation at controlling splenomegaly.

iv. Nitrosourea

Nitrosureas, like alkylating agents, inhibit DNA repair proteins. They are used to treat non-Hodgkin's lymphomas, multiple myeloma, malignant melanoma, in addition to brain tumors. A nitrosourea include but is not limited to a carmustine (BCNU), a lomustine (CCNU), a semustine (methyl-CCNU) or a streptozocin. Semustine has been used in such cancers as a primary brain tumor, a stomach or a colon cancer. Stroptozocin has been used to treat diseases such as a malignant pancreatic insulinoma or a malignant carcinoid. Streptozocin has beeen used to treat such cancers as a malignant melanoma, Hodgkin's disease and soft tissue sarcomas.

a. Carmustine

Carmustine (sterile carmustine) is one of the nitrosoureas used in the treatment of certain neoplastic diseases. It is 1,3 bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has been used in treating such cancers as a multiple myeloma or a malignant melanoma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is about 150 mg/m² to about 200 mg/m² intravenously every 6 weeks. This may be given as a single dose or divided into daily injections such as about 75 mg/m² to about 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention, for example about 10 mg/m², about 20 mg/m², about 30 mg/m², about 40 mg/m², about 50 mg/m², about 60 mg/m², about 70 mg/m², about 80 mg/m², about 90 mg/m² to about 100 mg/m².

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (about 0.05 mg/mL) and in absolute alcohol (about 70 mg/mL). Lomustine is relatively insoluble in water (less than about 0.05 mg/mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from about 30mg/m² to 100mg/m², about half of the radioactivity given was excreted in the form of degradation products within 24 hours. The serum half-life of the metabolites ranges from about 16 hours to about 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. Lomustine has been used to treat such cancers as small-cell lung cancer. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is about 130 mg/m² as a single oral dose every 6 weeks. In individuals with compromised bone marrow function, the dose should be reduced to about 100 mg/m² every 6 weeks. When lomustine is used in combination with other myelosuppressive drugs, the doses should be adjusted accordingly. It is understood that other doses may be used for example, about 20 mg/m², about 30mg/m², about 40 mg/m², about 50 mg/m², about 60 mg/m², about 70 mg/m², about 80 mg/m², about 90 mg/m², about 100 mg/m² to about 120 mg/m².

C. Triazine

A triazine include but is not limited to such drugs as a dacabazine (DTIC; dimethyltriazenoimidaz olecarboxamide), used in the treatment of such cancers as a malignant melanoma, Hodgkin's disease and a soft-tissue sarcoma.

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

i. Folic Acid Analogs

Folic acid analogs include but are not limited to compounds such as methotrexate (amethopterin), which has been used in the treatment of cancers such as acute lymphocytic leukemia, choriocarcinoma, mycosis fingoides, breast, head and neck, lung and osteogenic sarcoma.

ii. Pyrimidine Analogs

Pyrimidine analogs include such compounds as cytarabine (cytosine arabinoside), 5-fluorouracil (fluouracil; 5-FU) and floxuridine (fluorode-oxyuridine; FudR). Cytarabine has been used in the treatment of cancers such as acute granulocytic leukemia and acute lymphocytic leukemias. Floxuridine and 5-fluorouracil have been used in the treatment of cancers such as breast, colon, stomach, pancreas, ovary, head and neck, urinary bladder and topical premalignant skin lesions.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

iii. Purine Analogs and Related Inhibitors

Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia.

c. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

i. Mitotic Inhibitors

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP 16), tertiposide, paclitaxel, taxol, vinblastine, vincristine, and vinorelbine.

a. Epipodophyllotoxins

Epipodophyllotoxins include such compounds as tertiposide and VP16. VP16 is also known as etoposide and is used primarily for treatment of testicular tumors, in combination with bleomycin and cisplatin, and in combination with cisplatin for small-cell carcinoma of the lung. Tertiposide and VP16 are also active against cancers such as testis, other lung cancer, Hodgkin's disease, non-Hodgkin's lymphomas, acute granulocytic leukemia, acute nonlymphocytic leukemia, carcinoma of the breast, and Kaposi's sarcoma associated with acquired immunodeficiency syndrome (AIDS).

VP16 is available as a solution (e.g., 20 mg/ml) for intravenous administration and as 50 mg, liquid-filled capsules for oral use. For small-cell carcinoma of the lung, the intravenous dose (in combination therapy) is can be as much as about 100 mg/m² or as little as about 2 mg/m², routinely about 35 mg/m², daily for about 4 days, to about 50 mg/m², daily for about 5 days have also been used. When given orally, the dose should be doubled. Hence the doses for small cell lung carcinoma may be as high as about 200 mg/m² to about 250 mg/m². The intravenous dose for testicular cancer (in combination therapy) is about 50 mg/m² to about 100 mg/m² daily for about 5 days, or about 100 mg/m² on alternate days, for three doses. Cycles of therapy are usually repeated about every 3 to 4 weeks. The drug should be administered slowly (e.g., about 30 minutes to about 60 minutes) as an infusion in order to avoid hypotension and bronchospasm, which are probably due to the solvents used in the formulation.

b. Taxoids

Taxoids are a class of related compounds isolated from the bark of the ash tree, Taxus brevifolia. Taxoids include but are not limited to compounds such as docetaxel and paclitaxel.

Paclitaxel binds to tubulin (at a site distinct from that used by the vinca alkaloids) and promotes the assembly of microtubules. Paclitaxel is being evaluated clinically; it has activity against malignant melanoma and carcinoma of the ovary. In certain aspects, maximal doses are about 30 mg/m² per day for about 5 days or about 210 mg/m² to about 250 mg/m² given once about every 3 weeks.

c. Vince Alkaloids

Vince alkaloids are a type of plant alkaloid identified to have pharmaceutical activity. They include such compounds as vinblastine (VLB) and vincristine.

### 1. Vinblastine

Vinblastine is an example of a plant aklyloid that can be used for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. When the drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in about 7 days to about 10 days. If a moderate level of leukopenia (approximately 3000 cells/mm³) is not attained, the weekly dose may be increased gradually by increments of about 0.05 mg/kg of body weight. In regimens designed to cure testicular cancer, vinblastine is used in doses of about 0.3 mg/kg about every 3 weeks irrespective of blood cell counts or toxicity.

An important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, testis cancer, neuroblastoma, and Letterer-Siwe disease (histiocytosis X), as well as in carcinoma of the breast and choriocarcinoma in women.

Doses of about 0.1 mg/kg to about 0.3 mg/kg can be administered or about 1.5 mg/m² to about 2 mg/m² can also be administered. Alternatively, about 0.1 mg/m², about 0.12 mg/m², about 0.14 mg/m², about 0.15 mg/m², about 0.2 mg/m², about 0.25 , about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/m², about 10 mg/m², to about 20 mg/m², can be given.

### 2. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is about 0.4 mM.

Vinblastine and vincristine bind to plasma proteins. They are extensively concentrated in platelets and to a lesser extent in leukocytes and erythrocytes. Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than about 3 mg/dl (about 50 mM).

Vincristine sulfate is available as a solution (e.g., 1 mg/ml) for intravenous injection. Vincristine used together with corticosteroids is presently the treatment of choice to induce remissions in childhood leukemia; the optimal dosages for these drugs appear to be vincristine, intravenously, about 2 mg/m² of body-surface area, weekly; and prednisone, orally, about 40 mg/m², daily. Adult patients with Hodgkin's disease or non-Hodgkin's lymphomas usually receive vincristine as a part of a complex protocol. When used in the MOPP regimen, the recommended dose of vincristine is about 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, small cell lung, and carcinomas of the breast, bladder, and the male and female reproductive systems.

Doses of vincristine include about 0.01 mg/kg to about 0.03 mg/kg or about 0.4 mg/m² to about 1.4 mg/m² can be administered or about 1.5 mg/m² to about 2 mg/m² can also be administered. Alternatively, in certain embodiments, about 0.02 mg/m², about 0.05 mg/m², about 0.06 mg/m², about 0.07 mg/m², about 0.08 mg/m², about 0.1 mg/m², about 0.12 mg/m², about 0.14 mg/m², about 0.15 mg/m², about 0.2 mg/m², about 0.25 mg/m² can be given as a constant intravenous infusion.

d. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

### 1. Doxorubicin

Doxorubicin hydrochloride, 5,12-Naphthacenedione, (8s-cis)- 10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of diseases including ovarian, endometrial and breast tumors, bronchogenic oat-cell carcinoma, non-small cell lung carcinoma, stomach, genitourinary, thyroid, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma, soft tissue sarcoma, Ewing's sarcoma, rhabdomyosarcoma and acute lymphocytic leukemia. It is an alternative drug for the treatment of other diseases such as islet cell, cervical, testicular and adrenocortical cancers. It is also an immunosuppressant.

Doxorubicin is absorbed poorly and is preferably administered intravenously. The pharmacokinetics are multicompartmental. Distribution phases have half-lives of 12 minutes and 3.3 hours. The elimination half-life is about 30 hours, with about 40% to about 50% secreted into the bile. Most of the remainder is metabolized in the liver, partly to an active metabolite (doxorubicinol), but a few percent is excreted into the urine. In the presence of liver impairment, the dose should be reduced.

In certain embodiments, appropriate intravenous doses are, adult, about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by about 50% if the serum bilirubin lies between about 1.2 mg/dL and about 3 mg/dL and by about 75% if above about 3 mg/dL. The lifetime total dose should not exceed about 550 mg/m² in patients with normal heart function and about 400 mg/m² in persons having received mediastinal irradiation. In certain embodiments, and alternative dose regiment may comprise about 30 mg/m² on each of 3 consecutive days, repeated about every 4 week. Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

### 2. Daunorubicin

Daunorubicin hydrochloride, 5,12-Naphthacenedione, (8S-cis)-8-acetyl- 10-[(3-amino-2,3,6-trideoxy-a-L-lyxo-hexanopyranosyl)oxy]-7,8,9,10-tetrahydro-6,8,11-trihydroxy-10-methoxy-hydrochloride; also termed cerubidine and available from Wyeth. Daunorubicin (daunomycin; rubidomycin) intercalates into DNA, blocks DAN-directed RNA polymerase and inhibits DNA synthesis. It can prevent cell division in doses that do not interfere with nucleic acid synthesis.

In combination with other drugs it is often included in the first-choice chemotherapy of diseases such as, for example, acute granulocytic leukemia, acute myelocytic leukemia in adults (for induction of remission), acute lymphocytic leukemia and the acute phase of chronic myelocytic leukemia. Oral absorption is poor, and it preferably given by other methods (e.g., intravenously). The half-life of distribution is 45 minutes and of elimination, about 19 hours. The half-life of its active metabolite, daunorubicinol, is about 27 hours. Daunorubicin is metabolized mostly in the liver and also secreted into the bile (about 40%). Dosage must be reduced in liver or renal insufficiencies.

Generally, suitable intravenous doses are (base equivalent): adult, younger than 60 years, about 45 mg/m²/day (about 30 mg/m² for patients older than 60 year.) for about 1 day, about 2 days or about 3 days about every 3 weeks or 4 weeks or about 0.8 mg/kg/day for about 3 days, about 4 days, about 5 days to about 6 days about every 3 weeks or about 4 weeks; no more than about 550 mg/m² should be given in a lifetime, except only about 450 mg/m² if there has been chest irradiation; children, about 25 mg/m² once a week unless the age is less than 2 years, or the body surface less than about 0.5 m, in which case the weight-based adult schedule is used. It is available in injectable dosage forms (base equivalent) of about 20 mg (as the base equivalent to about 21.4 mg of the hydrochloride). Exemplary doses may be about 10 mg/m², about 20 mg/m², about 30 mg/m², about 50 mg/m², about 100 mg/m², about 150 mg/m², about 175 mg/m², about 200 mg/m2, about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

### 3. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed. Mitomycin has been used in tumors such as stomach, cervix, colon, breast, pancreas, bladder and head and neck.

In humans, mitomycin is rapidly cleared from the serum after intravenous administration. Time required to reduce the serum concentration by about 50% after a 30 mg. bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

### 4. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is often a component of first-choice combinations for treatment of diseases such as, for example, choriocarcinoma, embryonal rhabdomyosarcoma, testicular tumor, Kaposi's sarcoma and Wilms' tumor. Tumors that fail to respond to systemic treatment sometimes respond to local perfusion. Dactinomycin potentiates radiotherapy. It is a secondary (efferent) immunosuppressive.

In certain specific aspects, actinomycin D is used in combination with agents such as, for example, primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hours. The drug does not pass the blood-brain barrier. Actinomycin D is supplied as a lyophilized powder (0/5 mg in each vial). The usual daily dose is about 10 mg/kg to about 15 mg/kg; this is given intravenously for about 5 days; if no manifestations of toxicity are encountered, additional courses may be given at intervals of about 3 weeks to about 4 weeks. Daily injections of about 100 mg to about 400 mg have been given to children for about 10 days to about 14 days; in other regimens, about 3 mg/kg to about 6 mg/kg, for a total of about 125 mg/kg, and weekly maintenance doses of about 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

### 5. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of greater than about 35 mL per minute, the serum or plasma terminal elimination half-life of bleomycin is approximately 115 minutes. In patients with a creatinine clearance of less than about 35 mL per minute, the plasma or serum terminal elimination half-life increases exponentially as the creatinine clearance decreases. In humans, about 60% to about 70% of an administered dose is recovered in the urine as active bleomycin. In specific embodiments, bleomycin may be given by the intramuscular, intravenous, or subcutaneous routes. It is freely soluble in water. Because of the possibility of an anaphylactoid reaction, lymphoma patients should be treated with two units or less for the first two doses. If no acute reaction occurs, then the regular dosage schedule may be followed.

In preferred aspects, bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), esophagus, lung and genitourinary tract, Hodgkin's disease, non-Hodgkin's lymphoma, skin, penis, cervix, and vulva. It has also been used in the treatment of lymphomas and testicular carcinoma.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

d. Hormones and Antagonists

Hormonal therapy may also be used in conjunction with the present invention and/or in combination with any other cancer therapy or agent(s). The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### 1. Adrenocorticosteroids

Corticosteroid hormones are useful in treating some types of cancer (e.g., non-Hodgkin's lymphoma, acute and chronic lymphocytic leukemias, breast cancer, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

### 2. Other Hormones and Antagonists

Progestins such as hydroxyprogesterone caproate, medroxyprogesterone acetate, and megestrol acetate have been used in cancers of the endometrium and breast. Estrogens such as. diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

e. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention for use in combination therapies.

i. Platinum Coordination Complexes

Platinum coordination complexes include such compounds as carboplatin and cisplatin (cis-DDP). Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes, such as for example, intravenous, subcutaneous, intratumoral or intraperitoneal injection. Cisplatin can be used alone or in combination with other agents, with efficacious doses used in clinical applications of about 15 mg/m² to about 20 mg/m² for 5 days every three weeks for a total of three courses being contemplated in certain embodiments. Doses may be, for example, about 0.50 mg/m², about 1.0 mg/m², about 1.50 mg/m², about 1.75 mg/m², about 2.0 mg/m², about 3.0 mg/m², about 4.0 mg/m², about 5.0 mg/m², to about 10 mg/m².

ii. Other Agents

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

### 2. Radiotherapeutic Agents

Radiotherapeutic agents include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these agents effect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

### 3. Surgery

Approximately 60% of persons with cancer will undergo surgery of some type, which includes, for example, preventative, diagnostic or staging, curative and palliative surgery. Surgery, and in particular a curative surgery, may be used in conjunction with other therapies, such as the present invention and one or more other agents.

Curative surgery includes resection in which all or part of cancerous tissue is physically removed, excised and/or destroyed. It is further contemplated that surgery may remove, excise or destroy superficial cancers, precancers, or incidental amounts of normal tissue. Treatment by surgery includes for example, tumor resection, laser surgery, cryosurgery, electrosurgery, and miscopically controlled surgery (Mohs' surgery). Tumor resection refers to physical removal of at least part of a tumor. Upon excision of part of all of cancerous cells, tissue, or tumor, a cavity may be formed in the body.

Further treatment of the tumor or area of surgery may be accomplished by perfusion, direct injection or local application of the area with an additional anti-cancer agent. Such treatment may be repeated, for example, about every 1, about every 2, about every 3, about every 4, about every 5, about every 6, or about every 7 days, or about every 1, about every 2, about every 3, about every 4, or about every 5 weeks or about every 1, about every 2, about every 3, about every 4, about every 5, about every 6, about every 7, about every 8, about every 9, about every 10, about every 11, or about every 12 months. These treatments may be of varying dosages as well.

### 4. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. No. 5,686,072, U.S. Pat. No. 5,578,706, U.S. Pat. No. 4,792,447, U.S. Pat. No. 5,045,451, U.S. Pat. No. 4,664,911, and U.S. Pat. No. 5,767,072, each incorporated herein by reference). Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

a. Immune Stimulators

In a specific aspect of immunotherapy is to use an immune stimulating molecule as an agent, or more preferably in conjunction with another agent, such as for example, a cytokines such as for example IL-2, IL-4, IL-12, GM-CSF, tumor necrosis factor; interferons alpha, beta, and gamma; F42K and other cytokine analogs; a chemokine such as for example MIP-1, MIP-1beta, MCP-1, RANTES, IL-8; or a growth factor such as for example FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

Another cytokine specifically contemplate is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

b. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. For example, human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers.

c. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton et al., 1992; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

d. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

### 5. Genetic Therapy Agents

A tumor cell resistance to agents, such as chemotherapeutic and radiotherapeutic agents, represents a major problem in clinical oncology. One goal of current cancer research is to find ways to improve the efficacy of one or more anti-cancer agents by combining such an agent with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that gene therapy could be used similarly in conjunction with the headpin nucleic acid construct or proteinaceous molecule and/or other agents.

a. Inducers of Cellular Proliferation

In one embodiment of the present invention, it is contemplated that anti-sense mRNA directed to a particular inducer of cellular proliferation is used to prevent expression of the inducer of cellular proliferation. The proteins that induce cellular proliferation further fall into various categories dependent on function. The commonality of all of these proteins is their ability to regulate cellular proliferation.

For example, a form of PDGF, the sis oncogene, is a secreted growth factor. Oncogenes rarely arise from genes encoding growth factors, and at the present, sis is the only known naturally-occurring oncogenic growth factor.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

Other proteins such as Jun, Fos and Myc are proteins that directly exert their effects on nuclear functions as transcription factors.

b. Inhibitors of Cellular Proliferation

In certain embodiment, the restoration of the activity of an inhibitor of cellular proliferation through a genetic construct is contemplated. Tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1 /PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fins, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

In certain embodiments, it is contemplated that genetic constructs that stimulate apoptosis will be used to promote the death of diseased or undesired tissue. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

### 6. Other Biological Agents

It is contemplated that other agents may be used in combination with the present invention to improve the therapeutic efficacy of treatment. These additional agents include, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents such as for example, hyperthermia.

It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population.

In other embodiments, cytostatic or differentiation agents can be used in combination with the present invention to improve the anti-hyerproliferative efficacy of the treatments.

Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as, for example, the antibody c225, could be used in combination with the present invention to improve the treatment efficacy.

Another form of therapy for use in conjunction with the present invention and/or other agent(s) includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

IX. Pharmaceutical Compositions

A. Pharmaceutically Acceptable Carriers

Aqueous compositions of the present invention comprise an effective amount of the headpin protein, polypeptide, peptide, epitopic core region, agonist, antagonist, or such like, dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains an headpin agent as an active component or ingredient will be known to those of skill in the art in light of the present disclosure. Typically, such compositions can be prepared as injectables, either as liquid solutions or suspensions;. solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An headpin protein, polypeptide, peptide, agonist or antagonist of the present invention can be formulated into a composition in a neutral or salt form. Pharmaceutically acceptable salts, include the acid addition salts (formed with the free amino groups of the proteinaceous molecule) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The active protist headpin proteinaceous compostions or agents may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

B. Liposomes and Nanocapsules

In certain embodiments, the use of liposomes and/or nanoparticles is contemplated for the introduction of headpin protein, polypeptides, peptides or agents, or gene vectors, including both wild-type and antisense vectors, into host cells. The formation and use of liposomes is generally known to those of skill in the art, and is also described below.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

X. Kits

Certain embodiments of the present invention concerns diagnostic or therapeutic kits. The components of the various kits may be stored in suitable container means. The container means will generally include at least one vial, test tube, flask, bottle, syringe or other container means, into which the mammalian headpin proteinaceous molecule, gene, antibody or inhibitory formulation are placed, preferably, suitably allocated. The kits may also comprise a second container means for containing a sterile, pharmaceutically acceptable buffer or other diluent. The kits of the present invention will also typically include a means for containing the vials in close confinement for commercial sale, such as, e.g., injection or blow-molded plastic containers into which the desired vials are retained.

In one embodiment, a diagnostic kit may comprising headpin probes or primers for use with the nucleic acid detection methods. All the essential materials and reagents required for detecting headpin nucleic acid markers in a biological sample may be assembled together in a kit. This generally will comprise preselected primers for specific markers. Also included may be enzymes suitable for amplifying nucleic acids including various polymerases (RT, Taq, etc.), deoxynucleotides and buffers to provide the necessary reaction mixture for amplification.

Such kits generally will comprise, in suitable means, distinct containers for each individual reagent and enzyme as well as for each marker primer pair. Preferred pairs of primers for amplifying nucleic acids are selected to amplify the sequences specified in SEQ ID NO:1, SEQ ID NO:18 or SEQ ID NO:20, or a complement thereof.

In another embodiment, such kits will comprise hybridization probes specific for headpin corresponding to the sequences specified in SEQ ID NO:1, SEQ ID NO:18 or SEQ ID NO:20, or the complement thereof. Such kits generally will comprise, in suitable means, distinct containers for each individual reagent and enzyme as well as for each hybridization probe.

In other embodiments, the present invention concerns immunodetection kits for use with the immunodetection methods described above. As the headpin antibodies are generally used to detect wild-type or mutant headpin proteins, polypeptides or peptides, the antibodies will preferably be included in the kit. The immunodetection kits will thus comprise, in suitable container means, a first antibody that binds to a wild-type or mutant headpin protein, polypeptide or peptide, and optionally, an immunodetection reagent and further optionally, a wild-type or mutant headpin protein, polypeptide or peptide.

In preferred embodiments, monoclonal antibodies will be used. In certain embodiments, the first antibody that binds to the wild-type or mutant headpin protein, polypeptide or peptide may be pre-bound to a solid support, such as a column matrix or well of a microtitre plate.

The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

The kits may further comprise a suitably aliquoted composition of the wild-type or mutant headpin protein, polypeptide or polypeptide, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

Therapeutic kits of the present invention are kits comprising an headpin protein, polypeptide, peptide, biological functional equivalent, immunological fragment, domain, inhibitor, gene, vector, probe, primer, polynucleotide, nucleic acid, complement, antibody, or other headpin effector. Such kits will generally contain, in suitable container means, a pharmaceutically acceptable formulation of an headpin protein, polypeptide, peptide, biological functional equivalent, immunological fragment, domain, inhibitor, antibody, gene, polynucleotide, nucleic acid, complement, or vector expressing any of the foregoing in a pharmaceutically acceptable formulation. The kit may have a single container means, or it may have distinct container means for each compound.

When the components of the kit are provided in one or more liquid solutions, the liquid solution is an aqueous solution, with a sterile aqueous solution being particularly preferred. The headpin compositions may also be formulated into a syringeable composition. In which case, the container means may itself be a syringe, pipette, or other such like apparatus, from which the formulation may be applied to an infected area of the body, injected into an animal, or even applied to and mixed with the other components of the kit.

However, the components of the kit may be provided as dried powder(s). When reagents or components are provided as a dry powder, the powder can be reconstituted by the addition of a suitable solvent. It is envisioned that the solvent may also be provided in another container means.

The container means of the kits will generally include at least one vial, test tube, flask, bottle, syringe or other container means, into which the antibody may be placed, and preferably, suitably aliquoted. Where wild-type or mutant headpin protein, polypeptide or peptide, or a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

Irrespective of the number or type of containers, the kits of the invention may also comprise, or be packaged with, an instrument for assisting with the injection/administration or placement of the ultimate headpin proteinaceous molecule or nucleic acid composition within the body of an animal. Such an instrument may be a syringe, pipette, forceps, or any such medically approved delivery vehicle.

### XI. EXAMPLES

The following examples are included to demonstrate preferred embodiments of the invention. It should be appreciated by those of skill in the art that the techniques disclosed in the examples which follow represent techniques discovered to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cloning and Sequence Analysis of Headpin

Methods

Materials. All oligonucleotide primers were synthesized and cartridge purified by Genosys Biotechnologies, Inc. (The Woodlands, Tex.). Superscript II (GibcoBRL, Gaithersburg, Md.) reverse transcriptase was used in all reverse transcription reactions. Sequencing of differential display products was performed by using the Sequenase Kit (Amersham, Piscataway, N.J.). Sequencing of cDNA clones for sequence establishment and confirmation of headpin was performed by the MDACC DNA core facility. Differential display was performed using the Differential Display Kit (Display Systems Biotech, Vista, Calif.).

Cloning and sequence analysis of headpin. The differential display fragment corresponding to headpin was sequenced and GenBank was searched using the BLASTN program at the National Center for Biotechnology Information (NCBI) in the non-redundant database (http://www.ncbi.nlm.nih.gov). The GenBank search matched the fragment to 40 bp of a partial cDNA fragment HUR 7 (accession no. X98307, SEQ ID NO:16). A limited 5′ RACE (Boehringer-Mannheim, Indianapolis, IN) was performed using nested anti-sense primers to the combined headpin- HUR 7 sequence. Subsequently, a lambda gtl 1 phage human foreskin keratinocyte cDNA library (Clontech, Palo Alto, Calif.) was screened using the extended headpin cDNA as a probe. The positive plaques were selected and their cDNA were isolated, subcloned into a Topo TA Cloning kit system (Invitrogen, Carlsbad, Calif.), and sequenced. A full-length cDNA fragment covering the open reading frame was also generated by polymerase chain reaction (PCR™) and eight independent clones confirmed the sequence of headpin.

Results

Isolation and characterization of headpin cDNA. Five normal and five tumor patient-matched specimens from oral cavity squamous epithelium were analyzed using DDRT-PCR™. After subcloning and sequencing one of the differentially expressed fragments, a search of all NCBI/NIH nucleotide data banks was conducted. The differentially displayed fragment matched 40 bp at the 5′ end of a 405 base pair partial sequence called HUR 7 (SEQ ID NO:16) in the non-redundant database, which encodes an open reading frame, stop site and putative poly-adenylation tail (Abts et al., 1997). After performing a limited 5′ RACE to extend the open reading frame, a human keratinocyte cDNA was screened using a hybrid headpin-HUR 7 probe and obtained the remaining headpin sequence from a series of overlapping clones. Headpin diverges from HUR 7 at the 3′ end and contains a considerably longer open reading frame.

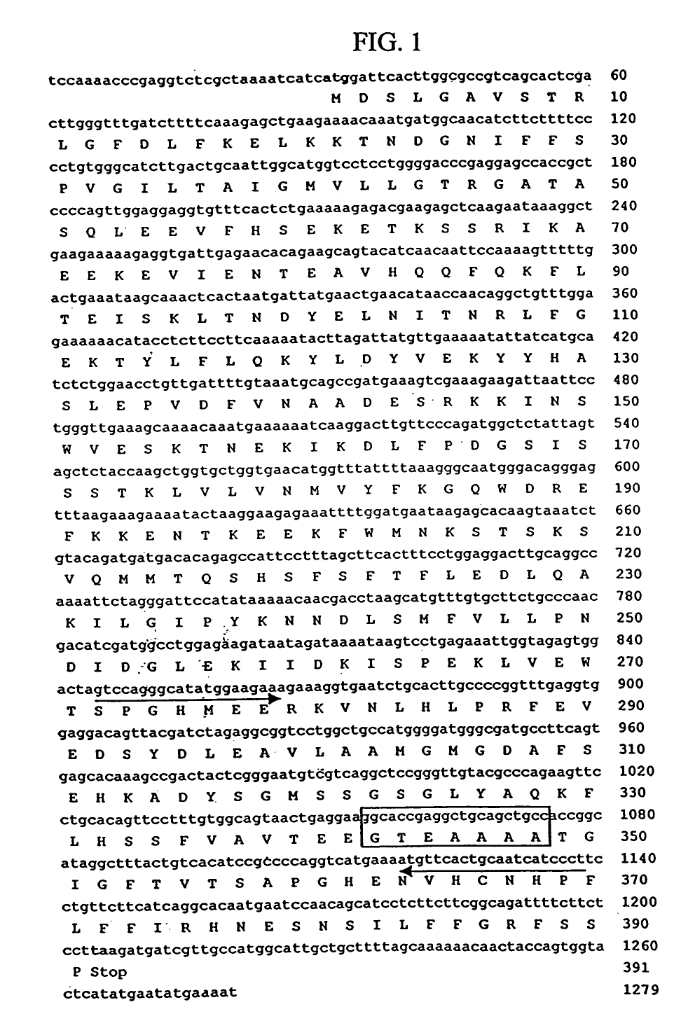

The coding sequence of the 1279 nucleotide headpin cDNA (SEQ ID NO:1) and corresponding amino acid sequence encoded therein (SEQ ID NO:2) are shown in FIG. 1. The predicted start methionine is located 30 bp downstream from the 5′ end of the cDNA fragment and is included in the single open reading frame of 391 amino acids (SEQ ID NO:2). Headpin has a predicted Mᵣ of 44 kDa and a calculated pI of 5.5.

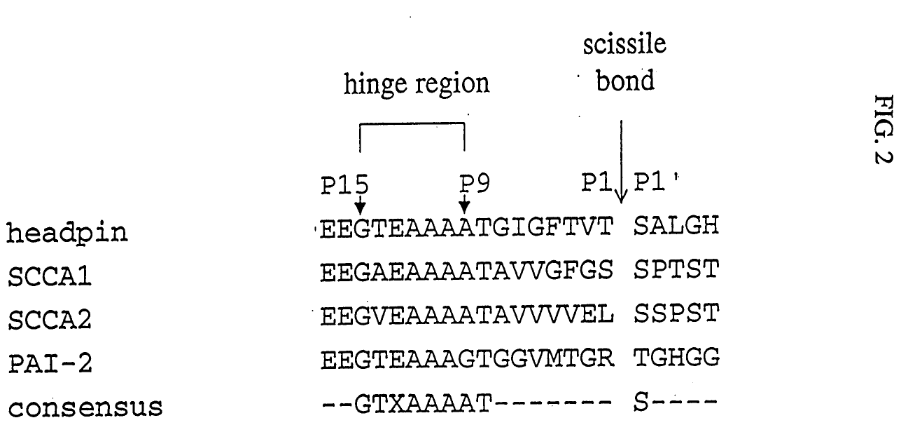

The reactive site loop (RSL) located approximately 30-50 amino acids from the carboxy-terminus is a variable region among serpins and is the domain which binds to the active site of serine proteinases (Potempa et al., 1994). Situated at the amino-terminus of the RSL is a highly conserved region known as the hinge region (P15-P9) (P-numbering system according to Schechter and Berger) which predicts whether or not a serpin behaves in an inhibitory capacity (Schechter and Berger, 1967). The consensus sequence for the hinge region that predicts whether a serpin belongs to the inhibitory class is GTXAAAAT (SEQ ID NO:17) where threonines are located at P14 and P8 (Schick et al., 1997). The predicted hinge region sequence for headpin is GTEAAAAT (FIG. 2, SEQ ID NO:12) and thus adheres strictly to the consensus sequence suggesting that it is an inhibitory serpin. Mutations in this region dramatically affect an inhibitors ability to function (Potempa et al., 1994). Another region in the RSL (known as the scissile bond site) located at the putative P1-P1′ of the inhibitory serpins involves the highly specific binding of the target proteinase. This specific covalent binding initiates a series of conformational changes that render the proteinase inactive (Schick et al., 1998). The scissile bond site of headpin is threonine and serine at P1 and P1′, respectively (FIG. 2). Among inhibitory serpins, serine often occupies the P1′ position (Schick et al., 1997).

Homology of headpin with ov-serpins. A comparison of amino acid sequences of headpin amino acid sequence (SEQ ID NO:2) and other serpins using the Clustal W protein comparison site at the European Bioinformatics Institute (www2.ebi.ac.uk/clustalw/) revealed 59%, 58%, and 42% overall sequence identity with other closely related ov-serpins SCCA1 (SEQ ID NO:9), SCCA2 (SEQ ID NO:10), and PAI-2 (SEQ ID NO:1) respectively. The sequences for the headpin variant (headpin^{var}) (SEQ ID NO:7) was also compared.

Interestingly, a variant of headpin was also cloned that contains the amino acid insert VRIKAEGKE (SEQ ID NO:13) located between amino acids 75 and 76. This insertion lies within helix C of the protein crystal structure of serpins and would be expected to affect the highly variable interhelical region between helices C and D (Remold-O'Donnell, 1993). The insertion is completely in frame and has been confirmed in full-length clones generated by RT-PCR™. HUR 7 does not conform to the standard serpin pattern because it lacks a hinge region, reactive site loop and prematurely truncates (SEQ ID NO:8). Many members of the ov-serpin family rely on an internal signal peptide rather than traditional signal sequences that allow individual serpins to be either secreted or remain within the cytoplasm. As with all ov-serpin family members, headpin has a penultimate serine residue rather than an asparagine and lacks a cleavable hydrophobic signal sequence found in the larger serpin superfamily (Remold-O'Donnell, 1980; Bartuski et al., 1997). Based on the features such as amino acid identity and structural lack of cleavable signal sequence, headpin is very likely a new member of the ov-serpin family (Remold-O'Donnell, 1993).

### Example 2

### Expression Patterns of Headpin

Methods

Relative RT-PCR™. Samples were obtained from normal and tumor site-matched biopsy tissue from five patients with squamous cell carcinomas. Tissue acquisition was obtained for a protocol of tumor banking which is approved by the institution's surveillance committee. The tissues were homogenized and the total RNA was extracted using Trizol Reagent (GibcoBRL). In addition, total RNA was also extracted from seven established head and neck tumor cell lines and primary cultures of normal oral epithelial cells. RT reactions of the biopsy tissue specimens were performed in duplicate using 2 μg of total RNA. Those of the cell lines were performed in single reactions using 2 μg of total RNA.

Probes specific for the 3′ end of headpin were chosen that had the least nucleotide identity to other closely-related serpins. The headpin probe sense primer was 5′-GTCCAGGGCATATGGAAGAA-3′ (SEQ ID NO:14) and the antisense was 5′-GGGATGATTGCAGTG-AACATT-3′ (SEQ ID NO:15). Prior to relative RT-PCR™, a PCR™ titration using two dilutions (1:20, 1:200) of the highest-expression biopsy specimen was performed to establish the PCR™ conditions that were in the linear range for detecting headpin. After the appropriate PCR™ cycle number was determined, relative RT-PCR™ was performed on duplicate RT reactions of biopsy specimen total RNA under the following PCR™ conditions; a hot start at 940° C. for 30 sec followed by 28 cycles of 94° C.×30 sec, 59° C×40 sec and 72° C. for 1 min and a 7 min extension at 72° C. The 18S competimer:primer system (Ambion, Austin, Tex.) designed to PCR™ amplify a fragment of 18S RNA was used as an internal control for normalization of PCR™ reactions. At a competimer:primer ratio of 8:2, the 18S product was also in linear range. Relative RT-PCR™ products were resolved on 6% polyacrylamide gels in 0.5 % Tris/Acetic acid/EDTA buffer run at 18 W. Stoffel Taq polymerase (Perkin-Elmer, Norwalk, Conn.) was used for the relative RT-PCR™.

Northern blot analysis. A human multiple tissue Northern blot filter containing 2 μg of poly (A)⁺ RNA was purchased from Clontech and hybridized in Rapidhyb buffer (Amersham) at 65° C. for 2.5 hrs. with headpin cDNA (base pairs 815-1107 of SEQ ID NO:1) radiolabeled with [α-³²P]dCTP. The filter was washed in 2×SSC/0.1%SDS at room temperature and then washed twice in 1×SCC/0.1%SDS at 65° C. An additional Northern blot was done to confirm transcript size and compare normal and tumor transcript expression. 20 μg of total RNA from a normal and patient matched squamous cell carcinoma was loaded onto a denatured polyacrylamide gel and run on a horizontal gel apparatus. The RNA was transferred to a nylon membrane overnight and the membrane was hybridized to the headpin probe.

Results

Northern blot and multiple tissue northern (MTN) blot analysis. Northern blot analysis was performed to establish the length of headpin RNA transcript in normal squamous epithelium. Total RNA from normal and tumor biopsy specimens derived from the oral cavity were hybridized to a headpin specific cDNA probe. Northern blot of 20 μg of total RNA extracted from tumor (T) and normal (N) biopsy specimens of the oral cavity. A 3.3 kb mRNA band was visible in the lane containing normal mucosa, but no signal was present from the tumor sample. This transcript size is comparable to the 3.0 kb mRNA species encoding maspin, but is longer than the 1.7 kb mRNA reported for SCCA1 (Jonson et al., 1999; Suminami et al., 1991). Northern blot of 2 μg of poly(A)⁺ RNA extracted from multiple human organs (heart, muscle, colon, thymus, spleen, kidney, liver, small intestine, brain, placenta, lung, PBLs) were probed with a headpin specific cDNA probe. The 12-lane MTN blot demonstrated no detectable hybridization signals from any of the different normal tissue types. Headpin transcript was also detected in normal skin by RT-PCR™.

Differential tissue expression of headpin. Differential expression of headpin was demonstrated by relative RT-PCR™ in normal and tumor site-matched biopsy specimens. Once headpin had been cloned and sequenced, headpin specific primers were chosen that would specifically amplify the headpin message from reverse transcribed total RNA obtained from the biopsy normal and tumor specimens. 18S RNA was used as a control. The results of relative RT-PCR™ data demonstrating the underexpression of headpin in tumor samples as compared to their normal counterparts. Reverse transcription reactions of the total mRNA from the tissue samples were run in duplicate. The expression pattern illustrates clearly that patient tumor biopsy samples, Ti, T2, T3 and T4 produce the headpin message in substantially lower amounts than their normal counterparts. However, one sample, T5, had equivalent or greater amount of headpin message than their normal counterpart. The mechanism for the loss of headpin in squamous carcinoma may be mutation, deletion or downregulation, but is presently unknown. Additionally, seven established head and neck tumor cell lines were examined and headpin was found expressed in significantly lower amounts as compared to the normal biopsy specimens based on the addition of equal amounts of cDNA in the RT-PCR™ reaction mixture. Presently, the consequences of headpin downregulation in squamous cell carcinoma of the head and neck is not known. However, one possibility is that headpin expression could be disadvantageous to tumor invasion mechanisms that rely on proteolytic degradation of extracellular matrix proteins.

Other experiments also indicate that headpin gene is transcriptionally regulated, rather than by methylation or mutation.

### Example 3

### Chromosomal Location of Headpin

Methods

Radiation hybrid (RH) mapping of headpin. To determine chromosome localization of the headpin gene, RH mapping was performed using the GeneBridge 4 RH panel (Research Genetics, Huntsville Ala.) with a set of primers located within the putative exon 8 (Schneider et al., 1995). Appropriate positive (donor) and negative (receptor) controls were performed before performing the PCR™-based assay and submitting the results to the Whitehead genome server at www-genome.wi.mit.edu/cgi-bin/contig/rhmapper.pl.

Results and Discussion

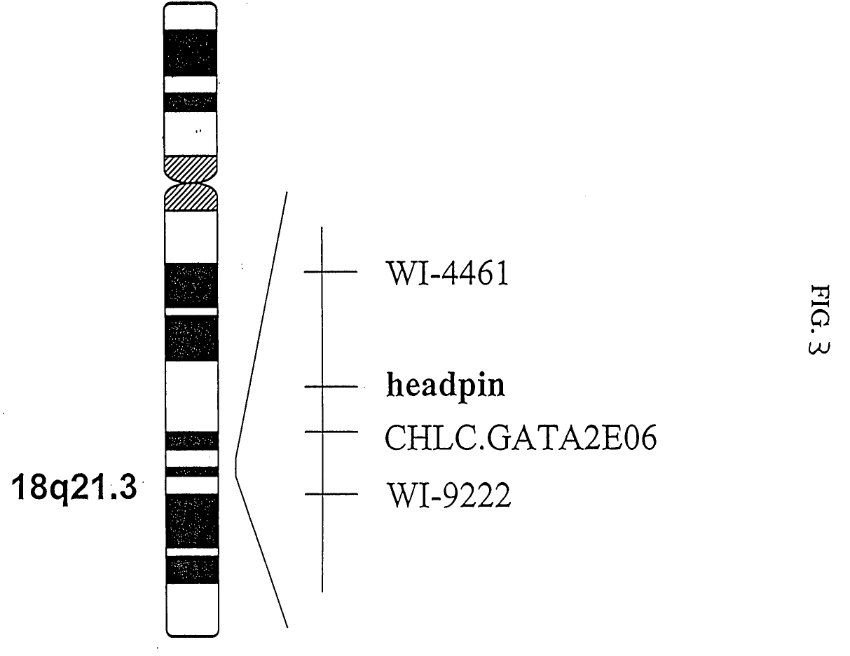

Localization of headpin to chromosome 18q. Radiation hybrid mapping was utilized to determine the chromosomal location of headpin. The results of the mapping study place headpin between the markers WI-4461 and CHLC.GATA2E06 which locates in the area of 18q21.3/18q22 (FIG. 3). This establishes headpin among the other ov-serpins such as SCCA1, SCCA2, plasminogen activator inhibitor-2 (PAI-2) and maspin (Bartuski et al., 1997). This area of chromosome 18 is actively studied because of the known breakpoints and LOH that occur on 18q in head and neck cancer and others (Jones et al., 1997; Zou et al., 1994; Pearlstein et al., 1998; Van Dyke et al., 1994; Papadimitrakopoulou et al., 1998). Already, four members of the ov-serpin family have been mapped to a 600 kb region within 18q21.3/18q22; cen-maspin, SCCA2, SCCA1, PAI-2-tel (Bartuski et al., 1997). Maspin, a class II tumor suppressor gene, is downregulated in mammary epithelial carcinoma (Lee et al., 1991). The loss of expression of maspin DNA is a consequence of regulatory changes rather than genomic changes (Sager et al., 1994). Headpin may represent an equivalent gene to maspin in head and neck cancer.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,196,265

U.S. Pat. No. 4,215,051

U.S. Pat. No. 4,275,149

U.S. Pat. No. 4,277,437

U.S. Pat. No. 4,366,241

U.S. Pat. No. 4,472,509

U.S. Pat. No. 4,554,101

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,664,911

U.S. Pat. No. 4,682,195

U.S. Pat. No. 4,683,195

U.S. Pat. No. 4,683,202

U.S. Pat. No. 4,684,611

U.S. Pat. No. 4,792,447

U.S. Pat. No. 4,800,159

U.S. Pat. No. 4,883,750

U.S. Pat. No. 4,946,773

U.S. Pat. No. 4,952,500

U.S. Pat. No. 5,021,236

U.S. Pat. No. 5,045,451

U.S. Pat. No. 5,057,414

U.S. Pat. No. 5,279,721

U.S. Pat. No. 5,302,523

U.S. Pat. No. 5,322,783

U.S. Pat. No. 5,384,253

U.S. Pat. No. 5,464,765

U.S. Pat. No. 5,538,877

U.S. Pat. No. 5,538,880

U.S. Pat. No. 5,550,318

U.S. Pat. No. 5,563,055

U.S. Pat. No. 5,578,706

U.S. Pat. No. 5,580,859

U.S. Pat. No. 5,589,466

U.S. Pat. No. 5,591,616

U.S. Pat. No. 5,602,253

U.S. Pat. No. 5,610,042

U.S. Pat. No. 5,656,610

U.S. Pat. No. 5,686,072

U.S. Pat. No. 5,702,932

U.S. Pat. No. 5,736,524

U.S. Pat. No. 5,767,072

U.S. Pat. No. 5,780,448

U.S. Pat. No. 5,789,215

U.S. Pat. No. 5,866,413

U.S. Pat. No. 5,925,553

U.S. Pat. No. 5,945,100

U.S. Pat. No. 5,981,274

U.S. Pat. No. 5,994,624

Abbondanzo et al., Breast Cancer Res. Treat., 16:182(#151), 1990.

Abts, Breuhahn, Michel, Koehrer, Esser, Ruzicka, Photochem and Photobiol 66:363-367, 1997.

Allred et al., Breast Cancer Res. Treat., 16:182(#149), 1990.

Angel et al., Cell, 49:729, 1987b.

Angel et al., Mol. Cell. Biol., 7:2256, 1987a.

Antibodies: A Laboratory Manual, Cold Spring Harbor Laboratory, 1988

Arap et al., Cancer Res., 55:1351-1354,1995.

Atchison and Perry, Cell, 46:253, 1986.

Atchison and Perry, Cell, 48:121, 1987.

Baichwal and Sugden, In: Gene Transfer, Kucherlapati R, ed., New York, Plenum Press, pp. 117-148, 1986.

Banerji et al., Cell, 27:299, 1981.

Banerji et al., Cell, 35:729, 1983.

Barrett, In: Proteinase Inhibitors, Ed. Barrett, A. J. et al, Elsevier, Amsterdam, pages 3-22, 1986.

Bartuski, Kamachi, Schick, Overhauser, Silverman, Genomics, 43:321-328, 1997.

Benvenisty and Neshif, Proc. Nat. Acad. Sci. USA, 83:9551-9555, 1986.

Berkhout et al., Cell, 59:273, 1989.

Blanar et al., EMBO J., 8:1139, 1989.

Blasi et al., In: Human Genes and Diseases, Ed. Blasi, F., John Wiley & Sons, Ltd., pages 377-414, 1986.

Bodine and Ley, EMBO J., 6:2997, 1987.

Boshart et al., Cell, 41:521, 1985.

Bosze et al., EMBO J., 5:1615, 1986.

Braddock et al., Cell, 58:269, 1989.

Brown et al., Breast Cancer Res. Treat., 16:192(#191), 1990.

Brutlag et al., CABIOS, 6:237-245, 1990.

Bulla and Siddiqui, J. Virol., 62:1437, 1986.

Caldas et al., Nat. Genet., 8:27-32,1994.

Campbell and Villarreal, Mol. Cell. Biol., 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campere and Tilghman, Genes and Dev., 3:537, 1989.

Campo et al., Nature, 303:77, 1983.

Carrell et al, Cold Spring Harbor Symp. Quant. Biol., 52:527-535, 1987.

Carrell et al., Trends Biochem. Sci., 10:20-24, 1985.

Carter and Flotte, Ann. N.Y. Acad. Sci., 770:79-90, 1995.

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chatterjee et al., Proc. Natl. Acad. Sci. USA., 86:9114, 1989.

Chatterjee, et al., Ann. N.Y. Acad. Sci., 770:79-90, 1995.

Chen and Okayama, Mol. Cell Biol., 7:2745-2752, 1987.

Cheng et al, Cancer Res., 54:5547-5551,1994.

Choi et al., Cell, 53:519, 1988.

Chou and Fasman, “Conformational Parameters for Amino Acids in Helical, β-Sheet, and Random Coil Regions Calculated from Proteins,” Biochemistry, 13(2):211-222, 1974b.

Chou and Fasman, “Empirical Predictions of Protein Conformation,” Ann. Rev. Biochem., 47:251-276, 1978b.

Chou and Fasman, “Prediction of b-Turns,” Biophys. J., 26:367-384, 1979.

Chou and Fasman, “Prediction of Protein Conformation,” Biochemistry, 13(2):222-245, 1974a.

Chou and Fasman, “Prediction of the Secondary Structure of Proteins from Their Amino Acid Sequence,” Adv. Enzymol. Relat. Areas Mol. Biol., 47:45-148, 1978a.

Coffin, In: Virology, ed., New York: Raven Press, pp. 1437-1500, 1990.

Cohen, Hirschhorn, Horowitz, Rubinstein, Polmar, Hong. and Martin, Jr., Proc. Natl. Acad. Sci. USA 75, 472-476, 1978.

Costa et al., Mol. Cell. Biol., 8:81, 1988.

Coupar et al., Gene, 68:1-10, 1988.

Cripe et al., EMBO J., 6:3745, 1987.

Culotta and Hamer, Mol. Cell. Biol., 9:1376, 1989.

Culver et al., In vivo gene transfer with retroviral vector-producer cells for treatment of experimental brain tumors. Science, 256:1550-1552, 1992.

Dandolo et al., J. Virology, 47:55, 1983.

Dayhoff, Atlas of Protein Sequence and Structure, Vol. 5, National Biomedical Research Foundation, Silver Spring, Md., 1972.

De Villiers et al., Nature, 312:242, 1984.

Deschamps et al., Science, 230:1174, 1985.

Dubensky et al., Proc. Nat. Acad. Sci. USA, 81:7529-7533, 1984.

Dvorak, New Eng. Journ. of Med. 315:1650-1659, 1986.

Edbrooke et al., Mol. Cell. Biol., 9:1908, 1989.

Edlund et al., Science, 230:912, 1985.

EPA 320,308

EPA 329,822

EPO 027,3085

Fechheimer et al., Proc. Natl. Acad. Sci. USA, 84:8463-8467, 1987.

Feng and Holland, Nature, 334:6178, 1988.

Ferkol et al., FASEB J., 7:1081-1091, 1993.

Ferrari et al., J. Virol., 70:3227-3234, 1996.

Fetrow and Bryant, “New Programs for Potein Tertiary Structure Prediction,” Biotech., 11:479-483, 1993.

Firak and Subramanian, Mol. Cell. Biol., 6:3667, 1986.

Fisher et al., J. Virol., 70:520-532, 1996.

Flotte et al., Proc. Natl. Acad. Sci. USA, 90:10613-10617, 1993.

Foecking M K, Hofstetter H. Gene. 45(1):101-105, 1986.

Fraley et al., Proc. Natl. Acad. Sci. USA, 76:3348-3352, 1979.

Frank, McClatchey, Devaney, Carey, Cancer Res., 57:824-827, 1997.

Fujita et al., Cell, 49:357, 1987.

GB 2202,328

Gefter et al., Somatic Cell Genet. 3:231-236, 1977.

Gerard et al, Mol. Biol. Med., 3:449-457, 1986.

Ghosh and Bachhawat, In: Liver diseases, targeted diagnosis and therapy using specific receptors and ligands, (Wu G, Wu C ed.), New York: Marcel Dekker, pp. 87-104, 1991.

Gilles et al., Cell, 33:717, 1983.

Gius et al., J. Virology, 62:665, 1988.

Gloss et al., EMBO J., 6:3735, 1987.

Godbout et al., Mol. Cell. Biol., 8:1169, 1988.

Goding, In: Monoclonal Antibodies: Principles and Practice, 2d ed., Orlando, Fla., Academic Press, pp. 60-61, 65-66, 71-74, 1986.

Goodbourn and Maniatis, Proc. Natl. Acad. Sci. USA, 85:1447, 1988.

Goodbourn et al., Cell, 45:601, 1986.

Goodman & Gilman's “The Pharmacological Basis of Therapeutics”

Goodman et al., Blood, 84:1492-1500, 1994.

Gopal, Mol. Cell Biol., 5:1188-1190, 1985.

Graham and Van Der Eb, Virology, 52:456-467, 1973.

Greene et al., Immunology Today, 10:272, 1989.

Grosschedl and Baltimore, Cell, 41:885, 1985.

Hartley, Ann. Rev. Biochem., 29:45-72, 1960.

Haslinger and Karin, Proc. Natl. Acad. Sci. USA., 82:8572, 1985.

Hauber and Cullen, J. Virology, 62:673, 1988.

Hay et al., J. Mol. Biol., 175:493-510, 1984.

Hearing and Shenk, J. Mol. Biol. 167:809-822, 1983.

Hearing et al., J. Virol., 67:2555-2558, 1987.

Hen et al., Nature, 321:249, 1986.

Hensel et al., Lymphokine Res., 8:347, 1989.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Hollstein et al., Science, 253:49-53, 1991.

Horlick and Benfield, Mol. Cell. Biol., 9:2396, 1989.

Huang et al., Cell, 27:245, 1981.

Hunt and Dayhoff, Biochem. Biophys. Res. Commun., 95:864-871, 1980.

Hussussian et al., Nature Genetics, 15-21, 1994.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Inouye et al., Nucleic Acids Res., 13:3101-3109, 1985.

Irie & Morton, Proc Natl Acad Sci USA. 83(22):8694-8698, 1986.

Irie et al., Lancet, 8;1(8641):786-787, 1989.

Jakobovits et al., Mol. Cell. Biol., 8:2555, 1988.

Jameel and Siddiqui, Mol. Cell. Biol., 6:710, 1986.

James, In: Proteolysis and Physiological Regulation, Ed. Ribbons, D. W. et al, Academic Press, New York, pages 125-142, 1976.

Jameson and Wolf, “The Antigenic Index: A Novel Algorithm for Predicting Antigenic Determinants,” Comput. Appl. Biosci., 4(1):181-186, 1988.

Jaynes et al., Mol. Cell. Biol., 8:62, 1988.

Johnson et al., “Peptide Turn Mimetics,” In: Biotechnology and Pharmacy, Pezzuto et al., eds., Chapman and Hall, New York, 1993.

Johnson et al., Mol. Cell. Biol., 9:3393, 1989.

Jones, Raval, Beals, Worsham, Van Dyke, Esclamado, Wolf, Bradford, Miller, Carey, Arch. Otolaryngol. Head and Neck, 123:610-614, 1997.

Jonson, Gorunova, Dawiskiba, Andren-Sandberg, Stenman, ten Dijke, Johansson, Hoglund, Genes, Chromosomes & Cancer, 24:62-71, 1999.

Kadesch and Berg, Mol. Cell. Biol., 6:2593, 1986.

Kamb et al., Nature Genetics, 8:22-26,1994a.

Kamb et al., Science, 2674:436-440,1994b.

Kaneda et al., Science, 243:375-378, 1989.

Kaplitt et al., Nat. Genet., 8:148-153, 1994.

Karin et al., Mol. Cell. Biol., 7:606, 1987.

Katinka et al., Cell, 20:393, 1980.

Katinka et al., Nature, 290:720, 1981.

Kato et al, J. Biol. Chem., 266:3361-3364, 1991.

Kawamoto et al., Mol. Cell. Biol., 8:267, 1988.

Kessler et al., Proc. Natl. Acad. Sci. USA, 93:14082-14087, 1996.

Ketcham et al., In: Atlas of Protein Sequence and Structure, Ed. Dayhoff, M.o., pages 131-143, 1978.

Khuri et al., Nat Med. 6(8):879-885, 2000.

Kiledjian et al., Mol. Cell. Biol., 8:145, 1988.

Klamut et al., Mol. Cell. Biol., 10:193, 1990.

Klein et al., Nature, 327:70-73, 1987.

Koch et al., Mol. Cell. Biol., 9:303, 1989.

Koeberl et al., Proc. Natl. Acad. Sci. USA, 94:1426-1431, 1997.

Kohler and Milstein, Eur. J. Immunol., 6:511-519, 1976.

Kohler and Milstein, Nature, 256:495-497, 1975.

Kriegler and Botchan, In: Eukaryotic Viral Vectors, Y. Gluzman, ed., Cold Spring Harbor: Cold Spring Harbor Laboratory, New York, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al., eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Larsen et al., Proc. Natl. Acad. Sci. USA., 83:8283, 1986.

Laskowski et al, Ann. Rev. Biochem., 49:593-626, 1980.

Laskowski et al, Ann. Rev. Biochem., 49:593-626, 1980.

Laskowski et al., Cold Spring Harbor Svmp. Quant. Biol., LII:545-553, 1987.

Laspia et al., Cell, 59:283, 1989.

Latimer et al., Mol. Cell. Biol., 10:760, 1990.

Lee et al., Nature, 294:228, 1981.

Lee, Tomasetto, Sager, Proc. Natl. Acad. Sci. USA, 88:2825, 1991.

Levin et al., Proc. Natl. Acad. Sci. USA, 80:6804-6808, 1983.

Levinson et al., Nature, 295:79, 1982.

Levrero et al., Gene, 101:195-202, 1991.

Lin et al., Mol. Cell. Biol., 10:850, 1990.

Luria et al., EMBO J., 6:3307, 1987.

Lusky and Botchan, Proc. Natl. Acad. Sci. USA., 83:3609, 1986.

Lusky et al., Mol. Cell. Biol., 3:1108, 1983.

Majors and Varmus, Proc. Natl. Acad. Sci. USA., 80:5866, 1983.

Mann et al., Cell, 33:153-159, 1983.

Markus, Takita, Camiolo, Corasanti, Evers, and Hobika, Cancer Res. 40:841-848, 1980. Mignatti, Robbins, and Rifkin, Cell 47:487-498, 1986.

McCown et al., Brain Res., 713:99-107, 1996.

McNeall et al., Gene, 76:81, 1989.

Miksicek et al., Cell, 46:203, 1986.

Mitchell et al, Proc Natl. Acad Sci. USA, 90, 11693-11697, 1993.

Mizukami et al., Virology, 217:124-130, 1996.

Mordacq and Linzer, Genes and Dev., 3:760, 1989.

Moreau et al., Nucl. Acids Res., 9:6047, 1981.

Morton et al., “Improved survival of patients with metastatic melanoma (MM) receiving active immunotherapy,” American Surgical Association, p. 83, 1992.

Muesing et al., Cell, 48:691, 1987.

Nakamura et al., In: Enzyme Immunoassays: Heterogeneous and Homogeneous Systems, Chapter 27, 1987.

Ng et al., Nuc. Acids Res., 17:601, 1989.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Nobri et al., Nature (London), 368:753-756,1995.

Okamoto et al., Proc. Natl. Acad. Sci. USA, 91:11045-11049,1994.

Ondek et al., EMBO J., 6:1017, 1987.

Ornitz et al., Mol. Cell. Biol., 7:3466, 1987.

Ossowski and Reich, Cell 35:611-619, 1983.

Palniter et al., Nature, 300:611, 1982.

Papadimitrakopoulou, Oh, El-Naggar, Izzo, Clayman, Mao, Clin. Canc. Res., 4:539-544, 1998.

Papamokos et al, J. Mol. Biol., 158:515-537, 1982.

Paskind et al., Virology, 67:242-248, 1975.

PCT Application No. US87/00880

PCT Application No. US89/01025

Pearlstein, Benninger, Carey, Zarbo, Torres, Rybicki, Dyke, Genes, Chromosomes and Cancer 21:333-339, 1998.

Pech et al., Mol. Cell. Biol., 9:396, 1989.

Perales et al., Proc. Natl. Acad. Sci. 91:4086-4090, 1994.

Perez-Stable and Constantini, Mol. Cell. Biol., 10:1116, 1990.

Physicians Desk Reference”

Picard and Schaffner, Nature, 307:83, 1984.

Ping et al., Microcirculation, 3:225-228, 1996.

Pinkert et al., Genes and Dev., 1:268, 1987.

Ponta et al., Proc. Natl. Acad. Sci. USA., 82:1020, 1985.

Porton et al., Mol. Cell. Biol., 10:1076, 1990.

Potempa, Korzus, Travis, J. Biol. Chem., 269:15957-15960, 1994.

Potter et al., Proc. Nat. Acad. Sci. USA, 81:7161-7165, 1984.

Queen and Baltimore, Cell, 35:741, 1983.

Quinn et al., Mol. Cell. Biol., 9:4713, 1989.

Radler et al., Science, 275:810-814, 1997.

Ravindranath et al., “Role of gangliosides in active immunotherapy with melanoma vaccine,” Intern. Rev. Immuno., 7:303-329, 1991.

Read et al., In: Proteinase Inhibitors, Ed. Barrett, A. J., et al, Elsevier, Amsterdam, pages 301-336, 1986.

Read et al., In: Proteinase Inhibitors, Ed. Barrett, A. J., et al, Elsevier, Amsterdam, pages 301-336, 1986.

Redondo et al., Science, 247:1225, 1990.

Reisman and Rotter, Mol. Cell. Biol., 9:3571, 1989.

Remington's Pharmaceutical Sciences” 15th Edition, pages 1035-1038 and 1570-1580) Remold-O'Donnell, 1980

Remold-O'Donnell, FEBS Lett, 315:105-108, 1993.

Renan, Radiother. Oncol., 19:197-218, 1990.

Resendez Jr. et al., Mol. Cell. Biol., 8:4579, 1988.

Ridgeway, In: Vectors: A survey of molecular cloning vectors and their uses, Rodriguez R L, Denhardt D T, ed., Stoneham:Butterworth, pp. 467492, 1988.

Ripe et al., Mol. Cell. Biol., 9:2224, 1989.

Rippe et al., Mol. Cell Biol., 10:689-695, 1990.

Rittling et al., Nucl. Acids Res., 17:1619, 1989.

Rosen et al., Cell, 41:813, 1988.

Rosenberg et al, Hosp. Prac., 21:131-137, 1986.

Rosenberg et al., Ann Surg. 210(4):474-484; discussion 484-548, 1989

Roux et al., Proc. Natl Acad. Sci. USA, 86:9079-9083, 1989.

Sager, Sheng, Anisowicz, Sotiropoulou, Zou, Stenman, Swisshelm, Chen, Hendrix, Pemberton, Rafidi, Ryan, Cold Spring Harbor Symposia on Quantitative Biology, LIX:537-546, 1994.

Sakai et al., Genes and Dev., 2:1144, 1988.

Sambrook, Fritsch, Maniatis, In: Molecular Cloning: A Laboratory Manual 2 rev.ed., Cold Spring Harbor: Cold Spring Harbor Laboratory Press, 1989.

Samulski et al., J. Virol., 61(10):3096-3101, 1987.

Satake et al., J. Virology, 62:970, 1988.

Schaffner et al., J. Mol. Biol., 201:81, 1988.

Schechter and Berger, Biochem. Biophys. Res. Comm., 27:157, 1967.

Schick, Broemme, Bartuski, Uemura, Schechter, Silverman, Proc. Natl. Acad. Sci. USA, 95:13465-13470, 1998.

Schick, Kamachi, Bartuski, Cataltepe, Schecter, Pemberton, Silverman, J. Biol. Chem., 272:1849-1855, 1997.

Schneider, Schick, Fish, Miller, Pena, Treter, Hui, Silverman, Proc. Natl. Acad. Sci. USA, 92:3147-3151, 1995.

Searle et al., Mol. Cell. Biol., 5:1480, 1985.

Serrano et al., Nature, 366:704-707,1993.

Serrano et al., Science, 267:249-252,1995.

Sharp and Marciniak, Cell, 59:229, 1989.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Sommer et al, Biochem., 26:6407-6410, 1987.

Spalholz et al., Cell, 42:183, 1985.

Spandau and Lee, J. Virology, 62:427, 1988.

Spandidos and Wilkie, EMBO J., 2:1193, 1983.

Sprengers et al, Blood, 69:381-387, 1987.

Stephens and Hentschel, Biochem. J., 248:1, 1987.

Strassburger et al, FEBS Lett., 157:219-223, 1983.

Stuart et al., Nature, 317:828, 1985.

Stump et al, J. Biol. Chem., 261:12759-12766, 1986.

Sullivan and Peterlin, Mol. Cell. Biol., 7:3315, 1987.

Suminami, Kishi, Sekiguchi, Kato, Biochem. Biophy. Res. Commun. 181:51-58, 1991.

Suzuki et al, J. Biol. Chem., 262:611-616, 1987.

Swartzendruber and Lehman, J. Cell. Physiology, 85:179, 1975.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Thiesen et al., J. Virology, 62:614, 1988.

Tibbetts Cell, 12:243-249, 1977.

Travis et al, Ann. Rev. Biochem., 52:655-709, 1983.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tur-Kaspa et al., Mol. Cell Biol., 6:716-718, 1986.

Tyndall et al., Nuc. Acids. Res., 9:6231, 1981.

Unkeless, Gordon, and Reich, J. Exp. Med. 139:834-850, 1974.

Van Dyke, Worsham, Benninger, Krause, Baker, Wolf, Drumheller, Tilley, Carey, Genes, Chromosomes and Cancer, 9:192-206, 1994.

Vannice and Levinson, J. Virology, 62:1305, 1988.

Vasseur et al., Proc. Natl. Acad. Sci. USA., 77:1068, 1980.

Wagner et al., Proc. Natl. Acad. Sci. 87(9):3410-3414, 1990.

Wagner et al., Science, 260:1510-1513, 1993.

Wang and Calame, Cell, 47:241, 1986.

Watt et al., Proc. Natl Acad. Sci., 83(2): 3166-3170, 1986.

Weber et al., Cell, 36:983, 1984.

Weinberg, Science, 254:1138-1146, 1991.

Weinberger et al., Mol. Cell. Biol., 8:988, 1984.

Weinberger et al., Science, 228:740-742, 1985.

Winoto and Baltimore, Cell, 59:649, 1989.

WO 88/10315

WO 89/06700

WO 90/07641

Wolf et al., “An Integrated Family of Amino Acid Sequence Analysis Programs,” Comput. Appl. Biosci., 4(1):187-191, 1988.

Wong et al., Gene, 10:87-94, 1980.

Wu and Wu, Adv. Drug Delivery Rev., 12:159-167, 1993.

Wu and Wu, Biochem., 27:887-892, 1988.

Wu and Wu, J. Biol. Chem., 262:4429-4432, 1987.

Xiao et al., J. Virol., 70:8098-8108, 1996.

Yang et al., Proc. Natl. Acad. Sci USA, 87:9568-9572, 1990.

Young et al., NEnglJMed. 7;299(23):1261-1266, 1978.

Yutzey et al., Mol. Cell. Biol., 9:1397, 1989.

Zou, Anisowicz, Hendrix, Thor, Neveu, Sheng, Rafidi, Seftor, Sager, Science, 263:526-529, 1994.

What is claimed is:

1. An isolated polynucleotide encoding a polypeptide having an amino acid sequence of SEQ ID NO:2.

2. The polynucleotide of claim 1, wherein said polynucleotide comprises a nucleic acid sequence of SEQ ID NO:1 or a complement thereof.

3. The polynucleotide of claim 2, wherein said polynucleotide further comprises a promoter operable in eukaryotic cells.

4. The polynucleotide of claim 3, wherein said promoter is heterologous to the coding sequence.

5. A isolated and purified nucleic acid that hybridizes, under high stringency conditions, to a DNA segment comprising about 102 to 1279 bases of SEQ ID NO:1.

6. A nucleic acid of about 15 to about 5000 base pairs comprising at least 102 contiguous base pairs of SEQ ID NO:1, or the complement thereof.

7. A isolated and purified nucleic acid that hybridizes, under high stringency conditions, to a DNA segment comprising about 15 to 700 bases of SEQ ID NO:18.

8. A isolated and purified nucleic acid that hybridizes, under high stringency conditions, to a DNA segment comprising about 15 to 478 bases of SEQ ID NO:20.

9. A nucleic acid of about 15 to about 5000 base pairs comprising about 15 contiguous base pairs of SEQ ID NO:18 or SEQ ID NO:20, or the complement thereof.

10. The nucleic acid of claim 9, comprising about 20 contiguous base pairs of SEQ ID NO:18 or SEQ ID NO:20, or the complement thereof.

11. The nucleic acid of claim 9, comprising about 30 contiguous base pairs of SEQ ID NO:18 or SEQ ID NO:20, or the complement thereof.

12. The nucleic acid of claim 9, comprising about 50 contiguous base pairs of SEQ ID NO:18 or SEQ ID NO:20, or the complement thereof.

13. The nucleic acid of claim 9, comprising about 100 contiguous base pairs of SEQ ID NO:18 or SEQ ID NO:20, or the complement thereof.

14. The nucleic acid of claim 9, comprising about 150 contiguous base pairs of SEQ ID NO:18 or SEQ ID NO:20, or the complement thereof.

15. The nucleic acid of claim 9, comprising about 250 contiguous base pairs of SEQ ID NO:18 or SEQ ID NO:20, or the complement thereof.

16. The nucleic acid of claim 9, comprising about 478 contiguous base pairs of SEQ ID NO:18 or SEQ ID NO:20, or the complement thereof.

17. The nucleic acid of claim 9, comprising about 550 contiguous base pairs of SEQ ID NO:18, or the complement thereof.

18. The nucleic acid of claim 9, comprising 700 contiguous base pairs of SEQ ID NO:18, or the complement thereof.

19. A peptide comprising at least 34 contiguous amino acids of SEQ ID NO:2.

20. A polypeptide comprising at least 51 contiguous amino acids of SEQ ID NO:2.

21. A peptide comprising about 10 contiguous amino acids of SEQ ID NO:19 or SEQ ID NO:21.

22. The peptide of claim 21, comprising about 15 contiguous amino acids of SEQ ID NO:19 or SEQ ID NO:21.

23. The peptide of claim 21, comprising about 20 contiguous amino acids of SEQ ID NO:19 or SEQ ID NO:21.

24. The peptide of claim 21, comprising about 25 contiguous amino acids of SEQ ID NO:19 or SEQ ID NO:21.

25. The peptide of claim 21, comprising about 30 contiguous amino acids of SEQ ID NO:19 or SEQ ID NO:21.

26. The peptide of claim 21, comprising about 50 contiguous amino acids of SEQ ID NO:19 or SEQ ID NO:21.

27. An expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells.

28. The expression cassette of claim 27, wherein said promoter is heterologous to the coding sequence.

29. The expression cassette of claim 28, wherein said promoter is an inducible promoter.

30. The expression cassette of claim 28, wherein said expression cassette is contained in a viral vector.

31. The expression cassette of claim 28, wherein said viral vector is selected from the group consisting of a retroviral vector, an adenoviral vector, and adeno-associated viral vector, a vaccinia viral vector, and a herpesviral vector.

32. The expression cassette of claim 27, wherein said expression cassette further comprises a polyadenylation signal.

33. The expression cassette of claim 27, wherein said expression cassette comprises a second polynucleotide encoding a second polypeptide.

34. The expression cassette of claim 33, wherein said second polynucleotide is under the control of a second promoter.

35. The expression cassette of claim 27, wherein the polynucleotide is positioned, in reverse orientation, under the control of a promoter that directs expression of an antisense product.

36. A cell comprising an expression cassette comprising a polynucleotide encoding a polypeptide having the sequence of SEQ ID NO:2, SEQ ID NO:19 or SEQ ID NO:21, wherein said polynucleotide is under the control of a promoter operable in eukaryotic cells.

37. A hybridoma cell that produces a monoclonal antibody that binds immunologically to a polypeptide having the sequence of SEQ ID NO:2, SEQ ID NO:19 or SEQ ID NO:2 1, or an immunologic fragment thereof.

38. A monoclonal antibody that binds immunologically to a polypeptide having the sequence of SEQ ID NO:2, SEQ ID NO:19 or SEQ ID NO:21, or an immunologic fragment thereof.

39. The monoclonal antibody of claim 38, wherein the antibody further comprises a detectable label.

40. The monoclonal antibody of claim 39, wherein the label is selected from the group consisting of a fluorescent label, a chemiluminescent label, a radiolabel and an enzyme.

41. A polyclonal antisera, antibodies of which bind immunologically to a polypeptide having the sequence of SEQ ID NO:2, SEQ ID NO:19 or SEQ ID NO:21, or an immunologic fragment thereof.

42. A method of diagnosing a cancer comprising the steps of:
(i) obtaining a tissue sample from a subject; and
(ii) assessing the expression of a headpin nucleic acid or a headpin polypeptide in cells of said sample.

43. The method of claim 42, wherein said cancer is selected from the group consisting of head and neck, brain, lung, liver, spleen, kidney, lymph node, small intestine, pancreas, blood cells, colon, stomach, breast, endometrium, prostate, testicle, ovary, skin, esophagus, bone marrow and blood cancer.

44. The method of claim 42, wherein said cancer is head and neck cancer.

45. The method of claim 42, wherein said assessing comprises assaying for a headpin nucleic acid from said sample.

46. The method of claim 45, further comprising subjecting said sample to conditions suitable to amplify said nucleic acid.

47. The method of claim 42, wherein said assessing comprises contacting said sample with an antibody that binds immunologically to a headpin polypeptide or peptide.

48. The method of claim 47, further comprising subjecting proteins, polypeptides or peptides of said sample to ELISA.

49. The method of claim 42, further comprising the step of comparing the expression of a headpin nucleic acid, polypeptide or peptide with the expression of headpin in non-cancer samples.

50. The method of claim 42, wherein assessing involves evaluating the structure of the headpin gene or transcript.

51. A method for treating subject with cancer comprising the step of administering to said subject a headpin proteinaceous composition.

52. The method of claim 51, wherein said tumor cell is derived from a tissue selected from the group consisting of head and neck, brain, lung, liver, spleen, kidney, lymph node, small intestine, blood cells, pancreas, colon, stomach, breast, endometrium, prostate, testicle, ovary, skin, esophagus, bone marrow and blood tissue.

53. The method of claim 52, wherein the subject is a human.

54. A method for treating a subject with cancer comprising the step of administering to said subject a nucleic acid (i) encoding a headpin protein, polypeptide or peptide and (ii) a promoter active in eukaryotic cells, wherein said promoter is operably linked to the region encoding said headpin protein, polypeptide or peptide.

55. The method of claim 54, wherein said tumor cell is derived from a tissue selected from the group consisting of head and neck, brain, lung, liver, spleen, kidney, lymph node, small intestine, blood cells, pancreas, colon, stomach, breast, endometrium, prostate, testicle, ovary, skin, head and neck, esophagus, bone marrow and blood tissue.

56. The method of claim 54, wherein said promoter is an inducible promoter.

57. The method of claim 54, wherein said expression cassette is contained in a viral vector.

58. The method of claim 57, wherein said viral vector is selected from the group consisting of a retroviral vector, an adenoviral vector, and adeno-associated viral vector, a vaccinia viral vector, and a herpesviral vector.

59. The method of claim 54, wherein said expression cassette further comprises a polyadenylation signal.

60. The method of claim 54, wherein said expression cassette comprises a second polynucleotide encoding a second polypeptide.

61. The method of claim 60, wherein said second polynucleotide is under the control of a second promoter.

62. The method of claim 54, wherein the subject is a human.

63. A nucleic acid detection kit comprising, in suitable container means, at least a first isolated mammalian headpin nucleic acid segment and a detection reagent.

64. An immunodetection kit comprising, in suitable container means, at least a first antibody that binds to a mammalian headpin protein, polypeptide, or peptide and a detection reagent.

65. A method for identifying a candidate substance that modulates mammalian headpin activity, comprising the steps of:
(a) admixing a mammalian headpin composition that comprises a candidate substance, a serine protease, and a mammalian headpin protein, polypeptide or peptide; and
(b) determining the ability of said candidate substance to modulate the ability of said mammalian headpin protein, polypeptide or peptide to inhibit said serine protease.
